(19) United States
(12) Patent Application Publication
(10) Pub. No.: US 2021/0188982 A1
(43) Pub. Date: Jun. 24, 2021

(54) EXTENDED INTERVAL DOSING OF NATALIZUMAB

(71) Applicant: BIOGEN MA INC., Cambridge, MA (US)

(72) Inventors: Nolan Robert Campbell, Somerville, MA (US); Ih Chang, Wayland, MA (US); Bharath Kumar Kandadi Muralidharan, Burlington, MA (US); Ivan Alexandrov Nestorov, Acton, MA (US)

(21) Appl. No.: 16/759,306

(22) PCT Filed: Oct. 25, 2018
(86) PCT No.: PCT/US2018/057605

§ 371 Date: Apr. 24, 2020

Related U.S. Application Data
(60) Provisional application No. 62/577,671, filed on Oct. 26, 2017.
(60) Provisional application No. 62/608,048, filed on Dec. 20, 2017.
(60) Provisional application No. 62/717,543, filed on Aug. 10, 2018.
(60) Provisional application No. 62/750,184, filed on Oct. 24, 2018.

(51) Int. Cl. C07K 16/28; G01N 33/68

(52) U.S. Cl. CPC C07K 16/2839; A61K 2039/505; G01N 33/6854

(57) ABSTRACT

Provided herein, in some embodiments, are methods for reducing the risk of developing progressive multifocal leukemia in patients undergoing natalizumab therapy by switching to an extended interval dosing (EID) schedule.

### CROSS-REFERENCE TO RELATED APPLICATIONS

This application claims the benefit of priority to U.S. Provisional Application Nos. 62/750,184, filed Oct. 24, 2018, 62/717,543, filed Aug. 10, 2018; 62/608,048, filed Dec. 20, 2017; and 62/577,671, filed Oct. 26, 2017, the entire disclosures of which are incorporated by reference in the entirety and for all purposes.

### BACKGROUND

Natalizumab is a humanized monoclonal IgG4 antibody that inhibits the migration of lymphocytes throughout the blood-brain barrier by blocking very late antigen (VLA)-4 interactions with vascular cell adhesion molecules (VCAM)-1 and reducing inflammatory lesions. Progressive multifocal leukoencephalopathy (PML), an opportunistic infection caused by the John Cunningham virus (JCV) that only occurs in patients who are immunocompromised, has affected a small population of patients using natalizumab.

### SUMMARY

Provided herein are methods for improving the safety of chronic natalizumab therapy, e.g. reducing the risk of developing PML in patients at risk thereof, using extended interval dosing (EID) of natalizumab. The present disclosure shows that administration of natalizumab on an EID schedule (e.g., a single dose every at least 5 weeks) reduces the risk of developing PML, and can be as efficacious as standard interval dosing (SID, i.e. a single dose every 4 weeks). In particular embodiments, EID of natalizumab reduces the risk of developing PML in patients who tested seronegative for anti-JCV antibodies before the inception of natalizumab treatment but later tested seropositive for anti-JCV antibodies during the course of natalizumab treatment at the standard 4-week intervals. The methods herein reduce the risk of developing PML without substantially compromising efficacy. In some embodiments, the EID schedule has an interval of from at least 5 weeks to no more than 8 weeks. In some embodiments, the EID schedule has an interval of from at least 5 weeks to no more than 7 weeks. In some embodiments, for patients having a weight range of <100 kg, the EID schedule has an interval of no more than 6 weeks. In some embodiments, for patients having a weight range of <80 kg, the EID schedule has an interval of no more than 6 weeks. In some embodiments, for patients having a weight range of <60 kg, the EID schedule has an interval of no more than 7 weeks. In some embodiments, the EID schedule has an interval of no more than 6 weeks (e.g., from at least 5 weeks to no more than 6 weeks).

In one aspect, methods for improving the safety of chronic natalizumab therapy are provided, comprising determining whether a patient has at least one risk factor for PML, and in the presence of at least risk factor administering natalizumab to the patient on an extended interval dosing (EID) schedule of at least 5 week intervals. The patient may already be undergoing chronic treatment with natalizumab for a particular condition, or may be commencing treatment with natalizumab for the subject condition. In some embodiments, the risk factor comprises prior immunosuppression of said patient. In some embodiments, the risk factor comprises the presence of anti-JCV antibodies in said patient. In some embodiments, the risk factor comprises having an anti-JCV antibody index level (e.g. mean index level) greater than 0.9, greater than 1.0, greater than 1.1, greater than 1.2, greater than 1.3, greater than 1.4, or greater than 1.5. In some embodiments, the risk factor comprises the length of any previous natalizumab therapy, e.g., at least about 6, 12, 18, 24, 30, or 36 months. In some embodiments, the EID schedule has an interval of from at least 5 weeks to no more than 8 weeks. In some embodiments, the EID schedule has an interval of from at least 5 weeks to no more than 7 weeks. In some embodiments, for patients having a weight range of <100 kg, the EID schedule has an interval of no more than 6 weeks. In some embodiments, for patients having a weight range of <80 kg, the EID schedule has an interval of no more than 6 weeks. In some embodiments, for patients having a weight range of <60 kg, the EID schedule has an interval of no more than 7 weeks. In some embodiments, the EID schedule has an interval of no more than 6 weeks (e.g., from at least 5 weeks to no more than 6 weeks).

In particular embodiments, the methods comprise determining the anti-JCV antibody status in a patient who is undergoing or commencing chronic treatment with natalizumab, and if said patient is seropositive for JCV antibodies then administering natalizumab to said patient on an EID schedule of at least 5 week intervals. In some embodiments, the EID schedule has an interval of from at least 5 weeks to no more than 8 weeks. In some embodiments, the EID schedule has an interval of from at least 5 weeks to no more than 7 weeks. In some embodiments, for patients having a weight range of <100 kg, the EID schedule has an interval of no more than 6 weeks. In some embodiments, for patients having a weight range of <80 kg, the EID schedule has an interval of no more than 6 weeks. In some embodiments, for patients having a weight range of <60 kg, the EID schedule has an interval of no more than 7 weeks. In some embodiments, the EID schedule has an interval of no more than 6 weeks (e.g., from at least 5 weeks to no more than 6 weeks).

Additional aspects of the present disclosure provide methods of reducing risk of developing progressive multifocal leukemia (PML) in a subject, comprising identifying a low PML risk subject who has been receiving natalizumab therapy on a standard interval dosing (SID) schedule of 4-week intervals, determining whether the subject has at least one risk factor for PML, e.g. if they have switched from a low PML risk subject to a high PML risk subject during the natalizumab therapy, and if the subject has switched to a high PML risk subject, identifying the high PML risk subject for natalizumab therapy on an EID dosing schedule of at least 5-week intervals. In some embodiments, the EID schedule has an interval of from at least 5 weeks to no more than 8 weeks. In some embodiments, the EID schedule has an interval of from at least 5 weeks to no more than 7 weeks. In some embodiments, for patients having a weight range of <100 kg, the EID schedule has an interval of no more than 6 weeks. In some embodiments, for patients having a weight range of <80 kg, the EID schedule has an interval of no more than 6 weeks. In some embodiments, for patients having a weight range of <60 kg, the EID schedule has an interval of no more than 7 weeks. In some embodiments, the EID schedule has an interval of no more than 6 weeks (e.g., from at least 5 weeks to no more than 6 weeks).

Other aspects of the present disclosure provide methods of reducing risk of developing PML in a subject, comprising administering to a subject a therapeutically effective amount of natalizumab on a SID schedule of 4-week intervals, wherein the subject is a low PML risk subject, determining whether the subject has switched from a low PML risk subject to a high PML risk subject during the natalizumab therapy, and if the subject has switched to a high PML risk subject, administering to the subject a therapeutically effective amount of natalizumab on an EID schedule of at least 5-week intervals. In some embodiments, the EID schedule has an interval of from at least 5 weeks to no more than 8 weeks. In some embodiments, the EID schedule has an interval of from at least 5 weeks to no more than 7 weeks. In some embodiments, for patients having a weight range of <100 kg, the EID schedule has an interval of no more than 6 weeks. In some embodiments, for patients having a weight range of <80 kg, the EID schedule has an interval of no more than 6 weeks. In some embodiments, for patients having a weight range of <60 kg, the EID schedule has an interval of no more than 7 weeks. In some embodiments, the EID schedule has an interval of no more than 6 weeks (e.g., from at least 5 weeks to no more than 6 weeks).

Still other aspects of the present disclosure provide methods of reducing risk of developing PML in a subject, comprising identifying a subject for natalizumab therapy on an EID schedule of at least 5-week intervals, wherein the subject has tested seropositive for anti-JCV antibodies and has received natalizumab therapy on a SID schedule of 4-week intervals. In some embodiments, the EID schedule has an interval of from at least 5 weeks to no more than 8 weeks. In some embodiments, the EID schedule has an interval of from at least 5 weeks to no more than 7 weeks. In some embodiments, for patients having a weight range of <100 kg, the EID schedule has an interval of no more than 6 weeks. In some embodiments, for patients having a weight range of <80 kg, the EID schedule has an interval of no more than 6 weeks. In some embodiments, for patients having a weight range of <60 kg, the EID schedule has an interval of no more than 7 weeks. In some embodiments, the EID schedule has an interval of no more than 6 weeks (e.g., from at least 5 weeks to no more than 6 weeks).

Yet other aspects of the present disclosure provide methods of reducing risk of developing PML in a subject, comprising administering to a subject a therapeutically effective amount of natalizumab on an EID schedule of at least 5-week intervals, wherein the subject has tested seropositive for anti-JCV antibodies and has been receiving natalizumab therapy on a SID schedule of 4-week intervals. In some embodiments, the EID schedule has an interval of from at least 5 weeks to no more than 8 weeks. In some embodiments, the EID schedule has an interval of from at least 5 weeks to no more than 7 weeks. In some embodiments, for patients having a weight range of <100 kg, the EID schedule has an interval of no more than 6 weeks. In some embodiments, for patients having a weight range of <80 kg, the EID schedule has an interval of no more than 6 weeks. In some embodiments, for patients having a weight range of <60 kg, the EID schedule has an interval of no more than 7 weeks. In some embodiments, the EID schedule has an interval of no more than 6 weeks (e.g., from at least 5 weeks to no more than 6 weeks).

Further aspects of the present disclosure provide methods of reducing risk of developing PML in a subject, comprising identifying a subject who has tested seropositive for anti-JCV antibodies and has been receiving natalizumab therapy on a SID schedule of 4-week intervals, and administering to the subject a therapeutically effective amount of natalizumab on an EID schedule of at least 5-week intervals. In some embodiments, the EID schedule has an interval of from at least 5 weeks to no more than 8 weeks. In some embodiments, the EID schedule has an interval of from at least 5 weeks to no more than 7 weeks. In some embodiments, for patients having a weight range of <100 kg, the EID schedule has an interval of no more than 6 weeks. In some embodiments, for patients having a weight range of <80 kg, the EID schedule has an interval of no more than 6 weeks. In some embodiments, for patients having a weight range of <60 kg, the EID schedule has an interval of no more than 7 weeks. In some embodiments, the EID schedule has an interval of no more than 6 weeks (e.g., from at least 5 weeks to no more than 6 weeks).

In one aspect, the present invention provides a method of improving the safety of chronic natalizumab therapy in a patient in need thereof, comprising determining whether the patient has at least one risk factor for progressive multifocal encephalopathy (PML), and in the presence of said at least one risk factor administering natalizumab to the patient on an EID schedule at a dose of 300 milligrams having an interval of at least 5 weeks and no more than 7 weeks, where the patient is from about 40 kg to about 80 kg in weight. In some embodiments, for patients having a weight range of <80 kg, the EID schedule has an interval of no more than 6 weeks. In some embodiments, for patients having a weight range of <60 kg, the EID schedule has an interval of no more than 7 weeks. In some embodiments, the EID schedule has an interval of no more than 6 weeks (e.g., from at least 5 weeks to no more than 6 weeks).

In another aspect, the present invention provides a method of administering to a patient in need thereof a natalizumab therapy, the method comprising: administering the natalizumab therapy on an EID schedule at a dose of 300 milligrams and having an interval of at least 5 weeks and no more than 7 weeks, wherein: a. the patient has a weight range of from 40 kg to less than 80 kg; and b. the PML risk of the natalizumab therapy is reduced as compared to natalizumab therapy on an SID schedule. In some embodiments, for patients having a weight range of <80 kg, the EID schedule has an interval of no more than 6 weeks. In some embodiments, for patients having a weight range of <60 kg, the EID schedule has an interval of no more than 7 weeks. In some embodiments, the EID schedule has an interval of no more than 6 weeks (e.g., from at least 5 weeks to no more than 6 weeks).

In another aspect, the present invention provides a method of improving the safety of chronic natalizumab therapy in a patient in need thereof, comprising determining whether the patient has at least one risk factor for progressive multifocal encephalopathy (PML), and in the presence of said at least one risk factor administering natalizumab to the patient on an EID schedule at a dose equivalent to 3.75 to 7.5 mg natalizumab/kg patient body weight having an interval of at least 5 weeks and no more than 8 weeks. In some embodiments, the EID schedule has an interval of from at least 5 weeks to no more than 7 weeks. In some embodiments, for patients having a weight range of <100 kg, the EID schedule has an interval of no more than 6 weeks. In some embodiments, for patients having a weight range of <80 kg, the EID schedule has an interval of no more than 6 weeks. In some embodiments, for patients having a weight range of <60 kg, the EID schedule has an interval of no more than 7 weeks. In some embodiments, the EID schedule has an interval of no more than 6 weeks (e.g., from at least 5 weeks to no more than 6 weeks).

In another aspect, the present invention provides a method of administering to a patient in need thereof a natalizumab therapy, the method comprising: administering the natalizumab therapy on an EID schedule at a dose equivalent to 3.75 to 7.5 mg natalizumab/kg patient body weight and having an interval of at least 5 weeks and no more than 8 weeks, wherein the PML risk of the natalizumab therapy is reduced as compared to natalizumab therapy on an SID schedule. In some embodiments, the EID schedule has an interval of from at least 5 weeks to no more than 7 weeks. In some embodiments, for patients having a weight range of <100 kg, the EID schedule has an interval of no more than 6 weeks. In some embodiments, for patients having a weight range of <80 kg, the EID schedule has an interval of no more than 6 weeks. In some embodiments, for patients having a weight range of <60 kg, the EID schedule has an interval of no more than 7 weeks. In some embodiments, the EID schedule has an interval of no more than 6 weeks (e.g., from at least 5 weeks to no more than 6 weeks).

In another aspect, the present invention provides a method of administering to a patient in need thereof a natalizumab therapy, the method comprising: administering the natalizumab therapy on an SID schedule for 12 months, and then administering the natalizumab therapy on an EID schedule. In some embodiments, the EID schedule has an interval of from at least 5 weeks to no more than 8 weeks. In some embodiments, the EID schedule has an interval of from at least 5 weeks to no more than 7 weeks. In some embodiments, for patients having a weight range of <100 kg, the EID schedule has an interval of no more than 6 weeks. In some embodiments, for patients having a weight range of <80 kg, the EID schedule has an interval of no more than 6 weeks. In some embodiments, for patients having a weight range of <60 kg, the EID schedule has an interval of no more than 7 weeks. In some embodiments, the EID schedule has an interval of no more than 6 weeks (e.g., from at least 5 weeks to no more than 6 weeks).

In another aspect, the present invention provides a method of improving the safety of chronic natalizumab therapy in a patient in need thereof, comprising determining whether the patient has at least one risk factor for progressive multifocal encephalopathy (PML), and in the presence of said at least one risk factor administering natalizumab to the patient on an EID schedule comprising at least 5 week intervals. In some embodiments, said at least one risk factor comprises prior immunosuppression of the patient. In some embodiments, said at least one risk factor comprises or further comprises the presence of serum anti-JCV antibodies in the patient. In some embodiments, said at least one risk factor comprises an anti-JCV antibody index level (e.g. mean index level) greater than 0.9, greater than 1.0, greater than 1.1, greater than 1.2, greater than 1.3, greater than 1.4, or greater than 1.5. In some embodiments, the EID schedule has an interval of from at least 5 weeks to no more than 8 weeks. In some embodiments, the EID schedule has an interval of from at least 5 weeks to no more than 7 weeks. In some embodiments, for patients having a weight range of <100 kg, the EID schedule has an interval of no more than 6 weeks. In some embodiments, for patients having a weight range of <80 kg, the EID schedule has an interval of no more than 6 weeks. In some embodiments, for patients having a weight range of <60 kg, the EID schedule has an interval of no more than 7 weeks. In some embodiments, the EID schedule has an interval of no more than 6 weeks (e.g., from at least 5 weeks to no more than 6 weeks).

In some embodiments, said at least one risk factor comprises the presence of anti-JCV antibodies in the patient, said determining step comprises determining the anti-JCV antibody status of the patient, and if the patient is seropositive for JCV antibodies then administering natalizumab to the patient on an EID schedule of at least 5 week intervals (e.g., an EID schedule of at least 5 week to no more than 8 week intervals). In some embodiments, the EID schedule has an interval of from at least 5 weeks to no more than 7 weeks. In some embodiments, the method comprises administering natalizumab to the patient on an EID schedule of 6 week intervals. In some embodiments, the patient has an anti-JCV antibody index of greater than 0.9. In some embodiments, the patient has an anti-JCV antibody index level greater than 1.5.

In some embodiments, the at least one risk factor comprises the length of prior natalizumab treatment, and if the patient has undergone more than six months (or at least 12 months) of natalizumab therapy then the method comprises administering natalizumab to the patient on an EID schedule having an interval of at least 5 weeks. In some embodiments, said at least one risk factor comprises the length of prior natalizumab treatment, the patient has undergone more than six months (or at least 12 months) of natalizumab therapy, and the method comprises administering natalizumab to the patient on an EID schedule having an interval of at least 5 weeks. In some embodiments, the EID schedule has an interval of from at least 5 weeks to no more than 8 weeks. In some embodiments, the EID schedule has an interval of from at least 5 weeks to no more than 7 weeks. In some embodiments, for patients having a weight range of <100 kg, the EID schedule has an interval of no more than 6 weeks. In some embodiments, for patients having a weight range of <80 kg, the EID schedule has an interval of no more than 6 weeks. In some embodiments, for patients having a weight range of <60 kg, the EID schedule has an interval of no more than 7 weeks. In some embodiments, the EID schedule has an interval of no more than 6 weeks (e.g., from at least 5 weeks to no more than 6 weeks).

In some embodiments, the more than six months of natalizumab therapy is more than six months of natalizumab therapy on a SID schedule. In some embodiments, the interval of the EID schedule is from 5 weeks to 8 weeks. In some embodiments, the interval of the EID schedule is from 5 to 6 weeks. In some embodiments, the interval of the EID schedule is 6 weeks.

In some embodiments, the patient is less than about 120 kg in weight; or the patient is less than about 100 kg in weight; or the patient is less than about 80 kg in weight; or the patient is less than about 60 kg in weight; or the patient is from about 40 kg to less than about 120 kg in weight; or the patient is from about 40 kg to less than about 100 kg in weight; or the patient is from about 40 kg to less than about 80 kg in weight; or the patient is from about 40 kg to less than about 60 kg in weight.

In some embodiments, the patient is from about 40 kg to about 80 kg in weight and the EID schedule has an interval of at least 5 weeks and no more than 6 weeks. In some embodiments, the patient is from about 40 kg to about 80 kg in weight and the EID schedule has an interval of 6 weeks. In some embodiments, the patient is from about 40 kg to about 60 kg in weight and the EID schedule has an interval of 6 weeks.

In some embodiments, the EID schedule comprises a dose of 300 milligrams. In some embodiments, the EID schedule comprises a dose equivalent to 3.75 to 7.5 mg natalizumab/kg patient body weight. In some embodiments, the patient has an autoimmune disease. In some embodiments, the autoimmune disease is MS. In some embodiments, the autoimmune disease is an inflammatory bowel disease. In some embodiments, the autoimmune disease is Crohn's disease. In some embodiments, the patient is diagnosed with, or has, epilepsy.

In another aspect, the present invention provides a method of administering to a patient in need thereof a natalizumab therapy, the method comprising: administering the natalizumab therapy on an EID schedule, wherein the patient has a weight range of from 40 kg to less than 80 kg; and the PML risk of the natalizumab therapy is reduced as compared to natalizumab therapy on an SID schedule. In some cases, the EID schedule comprises a dose of 300 mg. In some embodiments, the EID schedule comprises a dose equivalent to 3.75 to 7.5 mg natalizumab/kg patient body weight. In some embodiments, the EID schedule has an interval of from at least 5 weeks to no more than 8 weeks. In some embodiments, the EID schedule has an interval of from at least 5 weeks to no more than 7 weeks. In some embodiments, for patients having a weight range of <80 kg, the EID schedule has an interval of no more than 6 weeks. In some embodiments, for patients having a weight range of <60 kg, the EID schedule has an interval of no more than 7 weeks. In some embodiments, the EID schedule has an interval of no more than 6 weeks (e.g., from at least 5 weeks to no more than 6 weeks).

In some of the aspects, embodiments, cases, or examples described herein the patient is from about 40 kg to about 80 kg in weight and the EID schedule has an interval of at least 5 weeks and no more than 6 weeks. In some embodiments, the patient is from about 40 kg to about 80 kg in weight and the EID schedule has an interval of 6 weeks. In some embodiments, the patient is from about 40 kg to about 60 kg in weight and the EID schedule has an interval of at least 5 weeks and no more than 7 weeks. In some embodiments, the patient is from about 40 kg to about 60 kg in weight and the EID schedule has an interval of 6 weeks.

In certain preferred aspects, embodiments, cases, or examples described herein, the EID schedule maintains a mean trough a4-integrin receptor saturation of greater than 60% in an EID patient population. In certain preferred aspects, embodiments, cases, or examples described herein, the EID schedule maintains a mean trough α4β1 integrin receptor saturation of greater than 60% in an EID patient population. In some of the aspects, embodiments, cases, or examples described herein, the EID schedule maintains a mean trough a4-integrin receptor saturation of greater than 50% in an EID patient population. In some of the aspects, embodiments, cases, or examples described herein, the EID schedule maintains a mean trough α4β1 integrin receptor saturation of greater than 50% in an EID patient population. In some of the aspects, embodiments, cases, or examples described herein, the EID schedule maintains a mean trough a4-integrin receptor saturation of greater than 55% in an EID patient population. In some of the aspects, embodiments, cases, or examples described herein, the EID schedule maintains a mean trough α4β1 integrin receptor saturation of greater than 55% in an EID patient population. In some of the aspects, embodiments, cases, or examples described herein, the EID schedule maintains a mean trough a4-integrin receptor saturation of greater than 65% in an EID patient population. In some of the aspects, embodiments, cases, or examples described herein, the EID schedule maintains a mean trough α4β1 integrin receptor saturation of greater than 65% in an EID patient population.

In some of the aspects, embodiments, cases, or examples described herein, the efficacy of the natalizumab therapy on the EID schedule is reduced by no more than 20% as compared to natalizumab therapy on an SID schedule. In some of the aspects, embodiments, cases, or examples described herein, the risk of a Gd+ lesion at week 48 (or week 72) of natalizumab therapy on the EID schedule is increased by no more than about 5%, or 10%, expected mean number of Gd+ lesions at week 48 (or week 72) of natalizumab therapy on the EID schedule is increased by no more than about 0.5, 0.65, or 1, and/or the cumulative probability of a clinical relapse at week 48 (or week 72) of natalizumab therapy on the EID schedule is increased by no more than about 5%, 10%, 15%, or 20%.

In some of the aspects, embodiments, cases, or examples described herein, the method comprises administering the natalizumab on the EID schedule for at least 6 months, at least 1 year, at least 18 months, at least 2 years, or at least 5 years. In some of the aspects, embodiments, cases, or examples described herein, the method comprises administering the natalizumab on the EID schedule for <72 weeks (e.g., from at least 6 months to less than 72 weeks).

### BRIEF DESCRIPTION OF THE DRAWINGS

FIG. 1A shows a plot of Kaplan-Meier estimates of time to PML over 72 months for anti-JCV antibody positive patients grouped according to dosing schedules in Example 1. Patients in the SID cohort (solid line) and patients in the EID cohort (dashed line) are shown.

FIG. 1B shows a plot of Kaplan-Meier estimates of time to PML for anti-JCV antibody positive patients without prior immunosuppressant treatment grouped according to dosing schedule described in Example 1. Patients in the SID cohort (solid line) and patients in the EID cohort (dashed line) are shown.

FIG. 1C shows a plot of Kaplan-Meier estimates of time to PML for anti-JCV antibody positive patients previously treated with an immunosuppressant grouped according to dosing schedule described in Example 1. Patients in the SID cohort (solid line) and patients in the EID cohort (dashed line) are shown.

FIG. 1D shows a plot of Kaplan-Meier estimates of time to PML over 120 months for anti-JCV antibody positive patients grouped according to dosing schedule described in Example 1. Patients in the SID cohort (solid line) and patients in the EID cohort (dashed line) are shown. Model includes age, sex, prior use of IS, EID/SID group, and calendar year at the start of natalizumab treatment as covariates. EID=extended interval dosing. IS=immunosuppressant. KM=Kaplan-Meier. PML=progressive multifocal leukoencephalopathy. SID=standard interval dosing. *EID vs SID. †Number of patients who were still in the study and did not have PML at the end of the specified time. ‡Cumulative number of PML cases at the end of the specified time.

FIG. 2A shows a plot of Kaplan-Meier estimates of time to PML over 72 months for anti-JCV antibody positive patients grouped according to dosing schedule described in Example 2. Patients in the SID cohort (solid line) and patients in the EID cohort (dashed line) are shown.

FIG. 2B shows a plot of Kaplan-Meier estimates of time to PML for anti-JCV antibody positive patients without prior immunosuppressant treatment grouped according to dosing schedule described in Example 2. Patients in the SID cohort (solid line) and patients in the EID cohort (dashed line) are shown.

FIG. 2C shows a plot of Kaplan-Meier estimates of time to PML for anti-JCV antibody positive patients previously treated with an immunosuppressant grouped according to dosing schedule described in Example 2. Patients in the SID cohort (solid line) and patients in the EID cohort (dashed line) are shown.

FIG. 2D shows a plot of Kaplan-Meier estimates of time to PML over 120 months for anti-JCV antibody positive patients grouped according to dosing schedule described in Example 2. Patients in the SID cohort (solid line) and patients in the EID cohort (dashed line) are shown. Model includes age, sex, prior use of IS, EID/SID group, and calendar year at the start of natalizumab treatment as covariates. EID=extended interval dosing. IS=immunosuppressant. KM=Kaplan-Meier. PML=progressive multifocal leukoencephalopathy. SID=standard interval dosing. *EID vs SID. †Number of patients who were still in the study and did not have PML at the end of the specified time. ‡Cumulative number of PML cases at the end of the specified time.

FIG. 3A shows a plot of Kaplan-Meier estimates of time to PML over 72 months for anti-JCV antibody positive patients grouped according to dosing schedule described in Example 3. Patients in the SID cohort (solid line) and patients in the EID cohort (dashed line) are shown.

FIG. 3B shows a plot of Kaplan-Meier estimates of time to PML over 120 months for anti-JCV antibody positive patients grouped according to dosing schedule described in Example 3. Patients in the SID cohort (solid line) and patients in the EID cohort (dashed line) are shown.

FIG. 3C shows a plot of Kaplan-Meier estimates of time to PML for anti-JCV antibody positive patients without prior immunosuppressant treatment grouped according to dosing schedule described in Example 3. Patients in the SID cohort (solid line) and patients in the EID cohort (dashed line) are shown.

FIG. 3D shows a plot of Kaplan-Meier estimates of time to PML for anti-JCV antibody positive patients previously treated with an immunosuppressant grouped according to dosing schedule described in Example 3. Patients in the SID cohort (solid line) and patients in the EID cohort (dashed line) are shown. Model includes age, sex, prior use of IS, EID/SID group, and calendar year at the start of natalizumab treatment as covariates. EID=extended interval dosing. IS=immunosuppressant. KM=Kaplan-Meier. PML=progressive multifocal leukoencephalopathy. SID=standard interval dosing. *EID vs SID. †Number of patients who were still in the study and did not have PML at the end of the specified time. ‡Cumulative number of PML cases at the end of the specified time.

FIG. 4 shows a patient flow diagram for primary, secondary, and tertiary PML risk analyses. For inclusion in any analysis, patients must have had no dosing gaps (defined as an interval >12 weeks between two consecutive infusions) or overdoses (defined as an interval <3 weeks between two consecutive infusions). EID=extended interval dosing. JCV=JC virus. SID=standard interval dosing. †At least one occurrence of dosing gap (interval >12 weeks between two consecutive infusions) or overdose (interval <3 weeks between two consecutive infusions). ‡Patients switched between SID and EID more than once.

FIG. 5 shows dosing history and risk factor information for individual EID PML cases. IS=immunosuppressant. NA=not available. *Patient did not meet primary analysis definition of EID based on having received >15 doses in final 18 months. Note that the first diamond for all 13 patients is the initial infusion. The remaining diamonds are SID infusions or EID infusions as indicated.

FIG. 6 shows a plot of Kaplan-Meier estimates of time to PML over 120 months for anti-JCV antibody positive patients grouped according to EID definition lb described in Example 6. Patients in the SID cohort (solid line) and patients in the EID cohort (dashed line) are shown. Model includes age, sex, prior use of IS, EID/SID group, and calendar year at the start of natalizumab treatment as covariates. EID=extended interval dosing. IS=immunosuppressant. KM=Kaplan-Meier. PML=progressive multifocal leukoencephalopathy. SID=standard interval dosing. *EID vs SID. (a) Number of patients who were still in the study and did not have PML at the end of the specified time. (b) Cumulative number of PML cases at the end of the specified time.

FIG. 7 shows a plot of Kaplan-Meier estimates of time to PML over 120 months for anti-JCV antibody positive patients grouped according to EID definition 2b described in Example 6. Patients in the SID cohort (solid line) and patients in the EID cohort (dashed line) are shown. Model includes age, sex, prior use of IS, EID/SID group, and calendar year at the start of natalizumab treatment as covariates. EID=extended interval dosing. IS=immunosuppressant. KM=Kaplan-Meier. PML=progressive multifocal leukoencephalopathy. SID=standard interval dosing. *EID vs SID. (a) Number of patients who were still in the study and did not have PML at the end of the specified time. (b) Cumulative number of PML cases at the end of the specified time.

FIG. 8 shows a schematic of a prospective study design described in Example 6.

FIG. 9 shows simulated mean natalizumab concentration-time profiles over 52 weeks categorized by varying SID and EID dosing regimens and weight ranges.

FIG. 10 is a box plot of simulated trough α-4 integrin saturation levels categorized by patient weight ranges.

FIG. 11 illustrates a fitted curve of probability of Gd+ lesion occurrence calculated as described in Example 6, model 1. CI=confidence interval; GD+=gadolinium-enhancing; Q4W=every 4 weeks; Q6W=every 6 weeks; Q12W=every 12 weeks.

FIG. 12 illustrates a fitted curve of probability of Gd+ lesion occurrence calculated as described in Example 6, model 2. CI=confidence interval; GD+=gadolinium-enhancing; Q4W=every 4 weeks; Q6W=every 6 weeks; Q12W=every 12 weeks.

FIG. 13 illustrates a fitted curve of probability of clinical relapse occurrence calculated as described in Example 6, model 3. CI=confidence interval; GD+=gadolinium-enhancing; Q4W =every 4 weeks; Q6W =every 6 weeks; Q12W =every 12 weeks.

FIGS. 14A-C illustrates the 3 planned analyses of PML risk and the definitions of EID and SID used in Example 7. Each hypothetical patient depicts 2 years of infusion history. A. Primary analysis: test effect of last 18 months of dosing hiostry on PML risk. Definition: EID-1°: ≤15 infusions in the last 18 months (548 days); SID-1°>15 infusions in the last 18 months (548 days). B. Secondary analysis: test effect of any prolonged period of EID on PML risk. Definition: EID-2°: an EID-2° infusion is an infusion preceded by ≤10 doses in the prior 365 days, EID-2° patients received consecutive EID-2° infusions for ≥6 months; an SID-2° infusion is an infusion preceded by >10 doses in the prior 365 days, SID-2° patients received consecutive SID-2° infusions for ≥6 months; C. Tertiary analysis: test effect of a dosing history consisting of primarily EID on PML risk. Definition: EID-3°: ≤10 infusions per year over entire treatment duration; SID-3°: >10 infusions per year over entire treatment duration. EID=extended interval dosing; PML=progressive multifocal leukoencephalopathy; SID=standard interval dosing.

FIG. 15 illustrates a patient flow diagram for primary, secondary, and tertiary PML risk analyses as described in Example 7. Abbreviations: EID=extended interval dosing; JCV=JC virus; SID=standard interval dosing. For inclusion in any analysis of Example 7, patients must have had no dosing gaps (defined as an interval >12 weeks between 2 consecutive infusions) or overdoses (defined as an interval <3 weeks between 2 consecutive infusions). *Enrolled number as of Jun. 1, 2017. †At least 1 occurrence of dosing gap (interval >12 weeks between 2 consecutive infusions) or overdose (interval <3 weeks between 2 consecutive infusions). ‡Patients switched between SID and EID more than once.

FIGS. 16A-C shows a plot of Kaplan-Meier estimates of the cumulative probability of PML in EID vs SID groups in the (A) primary, (B) secondary, and (3) tertiary analyses of Example 7.

FIG. 17 shows dosing history and risk factor information for individual EID PML cases. IS=immunosuppressant; NA=not available; *Patient did not meet primary analysis definition of EID based on having received >15 doses in final 18 months.

FIG. 18 shows NOVA study endpoints and assessments as described in Example 8. *EID group only. †Including TSQM (Treatment Satisfaction Questionnaire for Medication), Neuro-QoL (Quality of Life in Neurological Disorders) Fatigue, MSIS-29 (Multiple Sclerosis Impact Scale), and EQ-5D-5L (EuroQol 5-dimensional questionnaire). ARR=annualized relapse rate.

FIG. 19 illustrates a rationale for the study dose intervals described in Example 8. Shaded areas indicate ranges of SID and EID dosing intervals for the NOVA study. SID 1° and EID 1°, SID 2° and EID 2°, and SID 3° and EID 3° refer to the definitions of SID and EID in the primary, secondary, and tertiary analyses, respectively, on PML risk in the TOUCH analysis described in Zhovtis Ryerson L, et al. Presented at ACTRIMS; Feb. 1-3, 2018; San Diego, Calif. LB250. ADI=average dosing interval.

FIGS. 20A-B illustrates an analysis suggesting the benefits of studying PML risk in patients who switch from SID to EID. A. Treatment effects of natalizumab are greater after year 1. Patients without clinical and radiologic disease activity after 1 and 2 years of natalizumab treatment in AFFIRM. (Havrdová E, et al. Lancet Neurol. 2009; 8:254-260; 12) Absence of combined clinical and radiological measures was defined as no relapse, no progression of disability (sustained for 12 weeks), no Gd+ lesions, and no new or enlarging T2-hyperintense lesions. B. PML risk is low in the first year of treatment. Conditional probability of developing PML using the life-table method in each year of treatment with multiple imputation to account for missing data in a pooled cohort (n=21,696)12 of natalizumab-treated patients from 4 large, observational, open-label studies: STRATIFY, STRATA, TOP, and TYGRIS. The box highlights the risk of PML during the first year of treatment.

### DETAILED DESCRIPTION

Natalizumab, sold under the trade name TYSABRI® (BIOGEN®, MA), is an integrin receptor antagonist approved by the U.S. Food and Drug administration (FDA) for treatment of multiple sclerosis and Crohn's disease. The FDA approved standard dosing (SD) regime is 300 milligrams (mg) infused intravenously over approximately one hour, every four weeks. Among the population of patients who have received natalizumab therapy, there is a small subpopulation of patients who have developed progressive multifocal leukoencephalopathy (PML) (Plavina, T. et al. Ann Neurol 2014; 76:802-12). Without wishing to be bound by theory, it is hypothesized that the therapeutic efficacy of natalizumab and the side-effect of increased PML risk are both caused by natalizumab's inhibition of lymphocyte trafficking to the brain. Although it has been hypothesized that natalizumab risk and efficacy are closely related, the present inventors have surprisingly found that extending the interval at which patients receive a dose of natalizumab can increase the safety of natalizumab treatment without a concomitant decrease in efficacy. The analysis of the TOUCH database and the PML database described herein conclusively demonstrates that EID treatment is associated with a lower risk of PML than SID treatment in anti-JCV antibody-positive patients. Similar results are expected for patient subgroups having other risk factors, or combinations of risk factors for PML, such as anti-JCV antibody negative patients, patients having an unknown anti-JCV antibody status, patients that have an anti-JCV antibody index level of >0.9 or >1.5, patients having a prior history of immunosuppressant use, and/or patients having been treated with natalizumab on an SID schedule for an extended period of time (e.g., >6 months). The present disclosure provides methods for reducing the risk of PML, for example, in patients who having a high PML risk status and/or patients who switch from low PML risk status to high PML risk status, by extending the interval at which the patients receive a dose of natalizumab.

Extended interval dosing (EID) herein refers to the administration of natalizumab at intervals that extend beyond the standard interval dosing (SID) dosing schedule of 300 mg every 4 weeks. An EID schedule should not exceed 12 doses of natalizumab within a 12-month period (one month equals 30 days), and typically does not exceed 11 or 10 doses within a 12-month period (one month equals 30 days). Thus, a SID schedule should exceed 10 doses of natalizumab within a 12-month period, and typically exceeds 11 or 12 doses in a 12-month period. In some embodiments, the EID schedule interval for administering natalizumab (e.g., 300 mg dose) is at least 5 weeks (35 days). For example, the EID schedule interval may be at least 6 weeks, at least 7 weeks, at least 8 weeks, at least 9 weeks, or at least 10 weeks. In some embodiments, the EID schedule interval is 5-12 weeks. For example, the EID schedule interval may be 5-11 weeks, 5-10 weeks, 5-9 weeks, 5-8 weeks, 5-7 weeks, or 5-6 weeks. In some embodiments, the EID schedule interval is 5 weeks, 6 weeks, 7 weeks, 8 weeks, 9 weeks, 10 weeks, 11 weeks, or 12 weeks. In some embodiments, the EID schedule interval is 5 weeks and 1 day, 5 weeks and 2 days, 5 weeks and 3 days, 5 weeks and 4 days, 5 weeks and 5 days, or 5 weeks and 6 days. In some embodiments, the EID schedule interval is 6 weeks and 1 day, 6 weeks and 2 days, 6 weeks and 3 days, 6 weeks and 4 days, 6 weeks and 5 days, or 6 weeks and 6 days. In some embodiments, the EID schedule is 7 weeks and 1 day, 7 weeks and 2 days, 7 weeks and 3 days, 7 weeks and 4 days, 7 weeks and 5 days, or 7 weeks and 6 days. In some embodiments, the EID schedule interval is 8 weeks and 1 day, 8 weeks and 2 days, 8 weeks and 3 days, 8 weeks and 4 days, 8 weeks and 5 days, or 8 weeks and 6 days. In some embodiments, the EID schedule interval is 9 weeks and 1 day, 9 weeks and 2 days, 9 weeks and 3 days, 9 weeks and 4 days, 9 weeks and 5 days, or 9 weeks and 6 days. In some embodiments, the EID schedule interval is 10 weeks and 1 day, 10 weeks and 2 days, 10 weeks and 3 days, 10 weeks and 4 days, 10 weeks and 5 days, or 10 weeks and 6 days. In some embodiments, the EID schedule interval is 11 weeks and 1 day, 11 weeks and 2 days, 11 weeks and 3 days, 11 weeks and 4 days, 11 weeks and 5 days, or 11 weeks and 6 days.

In some embodiments, the interval of the EID schedule interval is greater than 4 weeks and less than 12 weeks. In some embodiments, the interval of the EID schedule is at least 5 weeks and less than 12 weeks. In some embodiments, the interval of the EID schedule is greater than 4 weeks and no more than about 11 weeks. In some embodiments, the interval of the EID schedule is at least 5 weeks and no more than about 11 weeks. In some embodiments, the interval of the EID schedule is greater than 4 weeks and no more than 8 weeks. In some embodiments, the interval of the EID schedule is at least 5 weeks and no more than 8 weeks. In some embodiments, the interval of the EID schedule is greater than 4 weeks and no more than 7 weeks. In some embodiments, the interval of the EID schedule is at least 5 weeks and no more than 7 weeks. In some embodiments, the interval of the EID schedule is greater than 4 weeks and no more than 6 weeks. In some embodiments, the interval of the EID schedule is at least 5 weeks and no more than 6 weeks.

In some embodiments, the interval of the EID schedule is dependent on patient weight. For example, the EID schedule interval may be from greater than 4 weeks to less than 10 weeks, or from greater than 4 weeks to no more than 8 weeks, for patients having a weight range of less than 60 kg. In some embodiments, the EID schedule interval may be from greater than 4 weeks to less than 10 weeks, or from greater than 4 weeks to no more than 8 weeks, for patients having a weight range of from 40 to 59 kg. As another example, the EID schedule interval may be from greater than 4 weeks to less than 8 weeks, or from greater than 4 weeks to no more than 8 weeks, for patients having a weight range of less than 60 kg. In some embodiments, the EID schedule interval may be from greater than 4 weeks to less than 7 weeks for patients having a weight range of from 40 to 59 kg. In some embodiments, the EID schedule interval may be from greater than 4 weeks to no more than 7 weeks for patients having a weight range of from 40 to 59 kg. In some embodiments, the EID schedule interval may be from greater than 4 weeks to less than 7 weeks for patients having a weight range of less than 60 kg. In some embodiments, the EID schedule interval may be from greater than 4 weeks to no more than 7 weeks for patients having a weight range of less than 60 kg. In some embodiments, for patients having a weight range of <60 kg or from 40 kg to less than 60 kg, the interval of the EID schedule is 6 weeks.

In some embodiments, the EID schedule interval may be from at least 5 weeks to less than 10 weeks, or from at least 5 weeks to no more than 8 weeks, for patients having a weight range of less than 60 kg. In some embodiments, the EID schedule interval may be from at least 5 weeks to less than 10 weeks, or from at least 5 weeks to no more than 8 weeks, for patients having a weight range of from 40 to 59 kg. In some embodiments, the EID schedule interval may be from at least 5 weeks to less than 7 weeks for patients having a weight range of from 40 to 59 kg. In some embodiments, the EID schedule interval may be from at least 5 weeks to no more than 7 weeks for patients having a weight range of from 40 to 59 kg. In some embodiments, the EID schedule interval may be from at least 5 weeks to less than 7 weeks for patients having a weight range of less than 60 kg. In some embodiments, the EID schedule interval may be from at least 5 weeks to no more than 7 weeks for patients having a weight range of less than 60 kg.

As another example, the EID schedule interval may be from greater than 4 weeks to less than 8 weeks for patients having a weight range of greater than 59 kg. In some embodiments, the EID schedule interval may be from greater than 4 weeks to less than 8 weeks for patients having a weight range of from 60 kg to less than 80 kg. In some embodiments, the EID schedule interval may be from at least 5 weeks to less than 8 weeks for patients having a weight range of greater than 59 kg. In some embodiments, the EID schedule interval may be from at least 5 weeks to less than 8 weeks for patients having a weight range of from 60 kg to less than 80 kg. In some embodiments, the interval of the EID schedule for patients having a weight range ≥60 kg is no more than 8 weeks. In some embodiments, the interval of the EID schedule for patients having a weight range ≥60 kg is no more than 7 weeks. In some embodiments, the interval of the EID schedule for patients having a weight range ≥60 kg is no more than 6 weeks.

As another example, the EID schedule interval may be from greater than 4 weeks to less than 7 weeks for patients having a weight range of at least 80 kg, or a weight range of from 80 kg to less than 100 kg. In some embodiments, the EID schedule may be from at least 5 weeks to less than 7 weeks for patients having a weight range of at least 80 kg, or a weight range of from 80 kg to less than 100 kg. As another example, the EID schedule interval may be from greater than 4 weeks to no more than 6 weeks for patients having a weight range of at least 100 kg, or a weight range of from 100 kg to less than 120 kg. In some embodiments, the EID schedule interval may be from at least 5 weeks to no more than 6 weeks for patients having a weight range of at least 100 kg, or a weight range of from 100 kg to less than 120 kg. In some embodiments, the interval of the EID schedule for patients having a weight range ≥80 kg is no more than 7 weeks. In some embodiments, the interval of the EID schedule for patients having a weight range ≥80 kg is no more than 6 weeks.

As another example, the EID schedule interval may be from greater than 4 weeks to no more than 6 weeks for patients having a weight range of less than 100 kg. In some embodiments, the EID schedule interval may be greater than 4 weeks to no more than 6 weeks for patients having a weight range of from 40 kg to less than 100 kg. In some embodiments, the EID schedule interval may be from at least 5 weeks to less than 7 weeks for patients having a weight range of less than 100 kg. In some embodiments, the EID schedule interval may be from at least 5 weeks to no more than 6 weeks for patients having a weight range of less than 100 kg. In some embodiments, the EID schedule interval may be from at least 5 weeks to no more than 6 weeks for patients having a weight range of from 40 kg to less than 100 kg. In some embodiments, the interval of the EID schedule for patients having a weight range ≥100 kg is no more than 6 weeks. In some embodiments, the interval of the EID schedule for patients having a weight range ≥100 kg is no more than 5 weeks.

As another example, the EID schedule interval may be from greater than 4 weeks to no more than 6 weeks for patients having a weight range of less than 120 kg. In some embodiments, the EID schedule interval may be from greater than 4 weeks to no more than 6 weeks for patients having a weight range of from 40 kg to less than 120 kg. In some embodiments, the EID schedule interval may be from at least 5 weeks to less than 7 weeks for patients having a weight range of less than 120 kg. In some embodiments, the EID schedule interval may be from at least 5 weeks to no more than 6 weeks for patients having a weight range of less than 120 kg. In some embodiments, the EID schedule interval may be from at least 5 weeks to no more than 6 weeks for patients having a weight range of from 40 kg to less than 120 kg. In some embodiments, the EID schedule interval may be about 5 weeks for patients having a weight range of from 40 kg to less than 120 kg.

As another example, the EID schedule interval may be from at least 5 weeks to no more than 6 weeks for patients having a weight range of less than 80 kg. As another example, the EID schedule interval may be from at least 5 weeks to no more than 6 weeks for patients having a weight range of from 40 kg to less than 80 kg. In some embodiments, the EID schedule interval may be from at least 5 weeks to less than 7 weeks for patients having a weight range of less than 80 kg. In some embodiments, the EID schedule interval may be from at least 5 weeks to less than 7 weeks for patients having a weight range of from 40 kg to less than 80 kg. In some embodiments, the EID schedule interval may be from at least 5 weeks to less than 8 weeks for patients having a weight range of less than 80 kg. In some embodiments, the EID schedule interval may be from at least 5 weeks to less than 8 weeks for patients having a weight range of from 40 kg to less than 80 kg.

As another example, the EID schedule interval may be from greater than 4 weeks to no more than 6 weeks for patients having a weight range of less than 80 kg. As another example, the EID schedule interval may be from greater than 4 weeks to no more than 6 weeks for patients having a weight range of from 40 kg to less than 80 kg. In some embodiments, the EID schedule interval may be from greater than 4 weeks to less than 7 weeks for patients having a weight range of less than 80 kg. In some embodiments, the EID schedule interval may be from greater than 4 weeks to less than 7 weeks for patients having a weight range of from 40 kg to less than 80 kg. In some embodiments, the EID schedule interval may be from greater than 4 weeks to less than 8 weeks for patients having a weight range of less than 80 kg. In some embodiments, the EID schedule interval may be from greater than 4 weeks to less than 8 weeks for patients having a weight range of from 40 kg to less than 80 kg.

As another example, the EID schedule interval may be from at least 5 weeks to no more than 8 weeks for patients having a weight range of less than 80 kg. As another example, the EID schedule interval may be from at least 5 weeks to no more than 8 weeks for patients having a weight range of from 40 kg to less than 80 kg. In some embodiments, the EID schedule interval may be from at least 5 weeks to less than 9 weeks for patients having a weight range of less than 80 kg. In some embodiments, the EID schedule interval may be from at least 5 weeks to less than 9 weeks for patients having a weight range of from 40 kg to less than 80 kg.

As another example, the EID schedule interval may be from greater than 4 weeks to no more than 8 weeks for patients having a weight range of less than 80 kg. As another example, the EID schedule interval may be from greater than 4 weeks to no more than 8 weeks for patients having a weight range of from 40 kg to less than 80 kg. In some embodiments, the EID schedule interval may be from greater than 4 weeks to less than 9 weeks for patients having a weight range of less than 80 kg. In some embodiments, the EID schedule interval may be from greater than 4 weeks to less than 9 weeks for patients having a weight range of from 40 kg to less than 80 kg.

As another example, the EID schedule interval may be from greater than 4 weeks to no more than 9 weeks for patients having a weight range of less than 80 kg. As another example, the EID schedule interval may be from greater than 4 weeks to no more than 9 weeks for patients having a weight range of from 40 kg to less than 80 kg. In some embodiments, the EID schedule interval may be from greater than 4 weeks to less than 10 weeks for patients having a weight range of less than 80 kg. In some embodiments, the EID schedule interval may be from greater than 4 weeks to less than 10 weeks for patients having a weight range of from 40 kg to less than 80 kg.

In some embodiments, the patient is <120 kg in weight. In some embodiments, the patient is from 40 kg in weight to <120 kg in weight. In some embodiments, the patient is <100 kg in weight. In some embodiments, the patient is from 40 kg in weight to <100 kg in weight. In some embodiments, the patient is <80 kg in weight. In some embodiments, the patient is from 40 kg in weight to <80 kg in weight. In some embodiments, the patient is <60 kg in weight. In some embodiments, the patient is from 40 kg in weight to <60 kg in weight.

In some embodiments, the EID schedule interval is selected to maintain a mean trough α4β1-integrin receptor saturation of greater than 60% (or greater than 50, 55%, 65%, or 70%), or at least about 60% (or at least about 50%, 55%, 65%, or 70%), during the EID dosing period in a population of patients in need of natalizumab treatment. In some cases, the EID schedule interval that maintains a referenced (greater than 50%, 55%, 60%, 65%, or 70%, or at least about 50%, 55%, 60%, 65%, or 70%) mean trough α4β1-integrin receptor saturation during the EID dosing period in a population of patients in need of natalizumab treatment is an EID schedule having an interval of from greater than 4 weeks to no more than 12 weeks. In some cases, the EID schedule interval that maintains a referenced mean trough α4β1-integrin receptor saturation during the EID dosing period in a population of patients in need of natalizumab treatment is an EID schedule having an interval of from at least 5 weeks, from greater than 4 weeks to less than 12 weeks, from greater than 4 weeks to no more than 12 weeks, from at least 5 weeks to less than 12 weeks, or from at least 5 weeks to no more than 12 weeks.

In some cases, the EID schedule interval that maintains a referenced mean trough α4β1-integrin receptor saturation during the EID dosing period in a population of patients in need of natalizumab treatment is an EID schedule having an interval of from greater than 4 weeks to less than 10 weeks, from greater than 4 weeks to no more than 10 weeks, from at least 5 weeks to less than 10 weeks, from at least 5 weeks to no more than 10 weeks, from greater than 4 weeks to less than 8 weeks, from greater than 4 weeks to no more than 8 weeks, from at least 5 weeks to less than 8 weeks, from at least 5 weeks to no more than 8 weeks, from at least 5 weeks to no more than 7 weeks, from at least 5 weeks to no more than 6 weeks, or 6 weeks.

In some cases, the EID schedule interval that maintains a referenced mean trough α4β1-integrin receptor saturation during the EID dosing period in a population of patients in need of natalizumab treatment is an EID schedule having an interval of from greater than 4 weeks to less than 7 weeks, from greater than 4 weeks to no more than 7 weeks, from at least 5 weeks to less than 7 weeks, from at least 5 weeks to no more than 7 weeks, from greater than 4 weeks to less than 6 weeks, from greater than 4 weeks to no more than 6 weeks, from at least 5 weeks to less than 6 weeks, or from at least 5 weeks to no more than 6 weeks. In some cases, the EID schedule that maintains a referenced mean trough α4β1-integrin receptor saturation during the EID dosing period in a population of patients in need of natalizumab treatment is an EID schedule having an interval of from greater than 4 weeks to less than 10 weeks, or from at least 5 weeks to less than 10 weeks, wherein the population of patients have a weight range of less than 60 kg (e.g., from 40 to 59 kg). In some cases, the EID schedule that maintains a referenced mean trough α4β1-integrin receptor saturation during the EID dosing period in a population of patients in need of natalizumab treatment is an EID schedule having an interval of from at least 5 weeks to less than 8 weeks, or from at least 5 weeks to no more than 7 weeks, wherein the population of patients have a weight range of less than 60 kg (e.g., from 40 to 59 kg).

In some cases, the EID schedule that maintains a referenced mean trough α4β1-integrin receptor saturation during the EID dosing period in a population of patients in need of natalizumab treatment is an EID schedule having an interval of from greater than 4 weeks to less than 8 weeks, or from at least 5 weeks to less than 8 weeks, wherein the population of patients have a weight range of less than 80 kg (e.g., from 40 to 79 kg). In some cases, the EID schedule that maintains a referenced mean trough α4β1-integrin receptor saturation during the EID dosing period in a population of patients in need of natalizumab treatment is an EID schedule having an interval of from greater than 4 weeks to no more than 8 weeks, or from at least 5 weeks to no more than 8 weeks, wherein the population of patients have a weight range of less than 80 kg (e.g., from 40 to 79 kg).

In some cases, the EID schedule that maintains a referenced mean trough α4β1-integrin receptor saturation during the EID dosing period in a population of patients in need of natalizumab treatment is an EID schedule having an interval of from greater than 4 weeks to less than 7 weeks, or from at least 5 weeks to less than 7 weeks, wherein the population of patients have a weight range of less than 80 kg (e.g., from 40 to 79 kg). In some cases, the EID schedule that maintains a referenced mean trough α4β1-integrin receptor saturation during the EID dosing period in a population of patients in need of natalizumab treatment is an EID schedule having an interval of from greater than 4 weeks to no more than 7 weeks, or from at least 5 weeks to no more than 7 weeks, wherein the population of patients have a weight range of less than 80 kg (e.g., from 40 to 79 kg).

In some cases, the EID schedule that maintains a referenced mean trough α4β1-integrin receptor saturation during the EID dosing period in a population of patients in need of natalizumab treatment is an EID schedule having an interval of from greater than 4 weeks to no more than 6 weeks, or from at least 5 weeks to no more than 6 weeks, wherein the population of patients have a weight range of less than 80 kg (e.g., from 40 to 79 kg). In some cases, the EID schedule that maintains a referenced mean trough α4β1-integrin receptor saturation during the EID dosing period in a population of patients in need of natalizumab treatment is an EID schedule having an interval of from greater than 4 weeks to less than 6 weeks, or from at least 5 weeks to less than 6 weeks, wherein the population of patients have a weight range of less than 80 kg (e.g., from 40 to 79 kg).

In some cases, the EID schedule that maintains a referenced mean trough α4β1-integrin receptor saturation of greater than 60%, or at least about 60% during the EID dosing period in a population of patients in need of natalizumab treatment is an EID schedule having an interval of from greater than 4 weeks to less than 7 weeks, or from at least 5 weeks to less than 7 weeks, wherein the population of patients have a weight range of less than 100 kg (e.g., from 40 to 100 kg). In some cases, the EID schedule that maintains a mean trough α4β1-integrin receptor saturation of greater than 60%, or at least about 60% during the EID dosing period in a population of patients in need of natalizumab treatment is an EID schedule having an interval of from greater than 4 weeks to no more than 6 weeks, or from at least 5 weeks to no more than 6 weeks, wherein the population of patients have a weight range of less than 100 kg (e.g., from 40 to 100 kg). In some cases, the EID schedule that maintains a mean trough α4β1-integrin receptor saturation of greater than 60%, or at least about 60% during the EID dosing period in a population of patients in need of natalizumab treatment is an EID schedule having an interval of from greater than 4 weeks to no more than 5 weeks, or an interval of about 5 weeks, wherein the population of patients have a weight range of less than 100 kg (e.g., from 40 to 100 kg).

In some cases, the EID schedule that maintains a mean trough α4β1-integrin receptor saturation of greater than 50%, or at least about 50% during the EID dosing period in a population of patients in need of natalizumab treatment is an EID schedule having an interval of from greater than 4 weeks to no more than 6 weeks, or from at least 5 weeks to no more than 6 weeks, wherein the population of patients have a weight range of less than 120 kg (e.g., from 40 to 120 kg). In some cases, the EID schedule that maintains a mean trough α4β1-integrin receptor saturation of greater than 60%, or at least about 60% during the EID dosing period in a population of patients in need of natalizumab treatment is an EID schedule having an interval of from greater than 4 weeks to no more than 5 weeks, or an interval of about 5 weeks, wherein the population of patients have a weight range of less than 120 kg (e.g., from 40 to 120 kg).

As will be appreciated, the standard FDA approved dose amount is 300 mg. Thus, in some embodiments, the SID dose amount, or the EID dose amount, and typically both the SID dose amount and the EID dose amount is 300 mg. Accordingly, in some cases, wherein the patient has a weight range of from 40 kg to 79 kg, the dose amount may be from 3.75 mg natalizumab/kg patient body weight to 7.5 mg natalizumab/kg patient body weight. Similarly, in some embodiments, the dose amount may be from 3.75 mg natalizumab/kg patient body weight to 7.5 mg natalizumab/kg patient body weight. In some cases, wherein the patient has a weight range of from 40 kg to 59 kg, the dose amount may be from 5 mg natalizumab/kg patient body weight to 7.5 mg natalizumab/kg patient body weight. Similarly, in some embodiments, the dose amount may be from 5 mg natalizumab/kg patient body weight to 7.5 mg natalizumab/kg patient body weight.

In some cases, wherein the patient has a weight range of from 40 kg to 99 kg, the dose amount may be from 3.03 mg natalizumab/kg patient body weight to 7.5 mg natalizumab/kg patient body weight. Similarly, in some embodiments, the dose amount may be from 3.03 mg natalizumab/kg patient body weight to 7.5 mg natalizumab/kg patient body weight. In some cases, wherein the patient has a weight range of from 40 kg to less than 120 kg, the dose amount may be from 2.50 mg natalizumab/kg patient body weight to 7.5 mg natalizumab/kg patient body weight. Similarly, in some embodiments, the dose amount may be from 2.50 mg natalizumab/kg patient body weight to 7.5 mg natalizumab/kg patient body weight.

In some embodiments, an EID schedule includes 15 doses or fewer over an 18-month period. In other embodiments, an EID schedule includes 10 doses or fewer over a 12-month period. In some embodiments, an EID schedule includes 10 doses or fewer per year over the duration of infusion history.

In some embodiments, an EID schedule includes at least 3 doses of natalizumab, each dose administered on an average of every 5-12 weeks. For example, an EID schedule may include the administration of a single dose of natalizumab (starting at Day 0) then every 5 weeks for at least 10 weeks or at least 15 weeks. In some embodiments, an EID schedule includes the administration of a single dose of natalizumab every 6 weeks for at least 12 weeks or at least 18 weeks. In some embodiments, an EID schedule includes the administration of a single dose of natalizumab every 7 weeks for at least 14 weeks or at least 21 weeks. In some embodiments, an EID schedule includes the administration of a single dose of natalizumab every 8 weeks for at least 16 weeks or at least 24 weeks. In some embodiments, an EID schedule includes the administration of a single dose of natalizumab every 9 weeks for at least 18 weeks or at least 27 weeks. In some embodiments, an EID schedule includes the administration of a single dose of natalizumab every 10 weeks for at least 20 weeks or at least 30 weeks. In some embodiments, an EID schedule includes the administration of a single dose of natalizumab every 11 weeks for at least 22 weeks or at least 33 weeks. In some embodiments, an EID schedule includes the administration of a single dose of natalizumab every 11 weeks for at least 22 weeks or at least 33 weeks. In some embodiments, an EID schedule includes the administration of a single dose of natalizumab every 12 weeks for at least 24 weeks or at least 36 weeks.

In some embodiments, an EID schedule is followed (is administered) over the course of at least 6 months. In some embodiments, an EID schedule is followed over the course of at least 12 months (1 year). In some embodiments, an EID schedule is followed over the course of at least 18 months. In some embodiments, an EID schedule is followed over the course of at least 24 months (2 years). In some embodiments, an EID schedule is followed over the course of at least 30 months. In some embodiments, an EID schedule is followed over the course of at least 36 months (3 years).

In some embodiments, an EID schedule includes natalizumab 300 mg IV infusion every 6 weeks (-2/+5 days), e.g., up to Week 72. In some embodiments, an SID schedule includes natalizumab 300 milligram (mg) intravenous (IV) infusion every 4 weeks (−2/+5 days), e.g., up to Week 72.

In some embodiments, the EID schedule includes a variable dosing schedule that alternates between at least two different intervals. As a non-limiting example, a first dose of natalizumab may be administered at day 0, a second dose may be administered at week 5, a third dose may be administered at week 12 (7 weeks following second dose), a fourth dose may be administered at week 17 (5 weeks following third dose), a fifth dose may be administered at week 24 (7 weeks following fourth dose), and so on, alternating between dosing at 5 weeks and 7 weeks. As another non-limiting example, a first dose of natalizumab may be administered at day 0, a second dose may be administered at week 6, a third dose may be administered at week 14 (8 weeks following second dose), a fourth dose may be administered at week 20 (6 weeks following third dose), a fifth dose may be administered at week 28 (8 weeks following fourth dose), and so on, alternating between dosing at 6 weeks and 8 weeks. As yet another non-limiting example, a first dose of natalizumab may be administered at day 0, a second dose may be administered at week 7, a third dose may be administered at week 13 (6 weeks following second dose), a fourth dose may be administered at week 20 (7 weeks following third dose), a fifth dose may be administered at week 26 (6 weeks following fourth dose), and so on, alternating between dosing at 7 weeks and 6 weeks.

As described herein, EID schedules are provided for increasing the safety of natalizumab therapy. In some embodiments, the EID schedules are provided for increasing the safety of chronic natalizumab therapy. Safety may be increased by reducing the risk of an adverse event as compared to SID. As an exemplary embodiment, the EID reduces the risk of PML. In some cases, the EID reduces the risk of PML, reduces the risk of inducing generation of anti-natalizumab antibodies, reduces the risk of patient sensitization to natalizumab, or a combination thereof. In some cases, the EID reduces the risk of loss of efficacy of natalizumab treatment due to the generation of anti-idiotypic antibodies to natalizumab in the patient.

In some embodiments, the risk of developing PML in a subject receiving natalizumab on an EID schedule described herein is reduced by at least 20% relative to the risk of developing PML in a subject receiving natalizumab therapy on a SID schedule of 4-week intervals. For example, the risk of developing PML in a subject receiving natalizumab on an EID schedule described herein is reduced by at least 30%, 40%, or 50% relative to the risk of developing PML in a subject receiving natalizumab therapy on a SID schedule of 4-week intervals.

In some embodiments, the risk of developing PML in a subject receiving natalizumab on an EID schedule described herein is reduced by at least 20% relative to the risk of developing PML in a subject receiving natalizumab therapy on a SID schedule, and the efficacy of the natalizumab therapy is reduced by less than 10% relative to the efficacy of SID. For example, the risk of developing PML in a subject receiving natalizumab on an EID schedule described herein is reduced by at least 30%, 40%, or 50% relative to the risk of developing PML in a subject receiving natalizumab therapy on a SID schedule of 4-week intervals, and the efficacy of the natalizumab therapy is reduced by less than 10% relative to the efficacy of SID.

In some embodiments, the risk of developing PML in a subject receiving natalizumab on an EID schedule of at least 5-week intervals is reduced by at least 20% relative to the risk of developing PML in a subject receiving natalizumab therapy on a SID schedule of 4-week intervals. For example, the risk of developing PML in a subject receiving natalizumab on an EID schedule of at least 5-week intervals is reduced by at least 30%, 40%, or 50% relative to the risk of developing PML in a subject receiving natalizumab therapy on a SID schedule of 4-week intervals.

A subject, as provided herein, is typically a male or female human subject (patient) who is undergoing or who will undergo treatment with natalizumab for a particular condition. The condition may be an autoimmune condition or an inflammatory condition. Often, autoimmune conditions are considered inflammatory conditions and vice versa, thus, in some embodiments the subject has an autoimmune condition and/or inflammatory condition. An autoimmune condition is a condition in which a subject's immune system attacks the subject's own cells/tissues. Non-limiting examples of autoimmune conditions include multiple sclerosis (MS) (e.g., relapsing-remitting MS, secondary progressive MS, and/or primary progressive MS), Crohn's disease, rheumatoid arthritis, lupus, celiac disease, Sjorgren's syndrome, Polymyalgia rheumatic, ankylosing spondylitis, Type 1 diabetes, alopecia areata, vasculitis, and temporal arteritis. Many of the foregoing conditions are also inflammatory conditions. Thus, in some embodiments, the methods of the present disclosure comprise identifying a subject for natalizumab therapy on an EID schedule, or administering to a subject natalizumab therapy on an EID schedule of at least 5-week intervals, wherein the subject is at high risk for PML and has an autoimmune condition. In some embodiments, the autoimmune condition is multiple sclerosis. In some embodiments, the autoimmune condition is Crohn's disease.

In some embodiments, the subject has been diagnosed with epilepsy. Epilepsy is a central nervous system disorder (neurological disorder) in which nerve cell activity in the brain becomes disrupted, causing seizures or periods of unusual behavior, sensations and sometimes loss of consciousness. Thus, in some embodiments, the methods of the present disclosure comprise identifying a subject for natalizumab therapy on an EID schedule, or administering to a subject natalizumab therapy on an EID schedule of greater than 4-week intervals (e.g., at least 5-week intervals), wherein the subject has epilepsy, has recently had a seizure, or has epilepsy and has recently had a seizure. In some embodiments, the subject is at high risk for PML and has epilepsy, has recently had a seizure, or has epilepsy and has recently had a seizure.

In some embodiments, a subject has a prior history of immunosuppression. In some embodiments, a subject was treated with an immunosuppressant prior to receiving natalizumab therapy on SID schedule of 4-week intervals.

A high PML risk subject is a subject who is seropositive for anti-JCV antibodies. In some embodiments, a high PML risk subject has had prior immunosuppression and is seropositive for anti-JCV antibodies. In some embodiments, a PML risk subject has an anti-JCV antibody index level (e.g., a mean index level) of greater than 1.5. In some embodiments, a low PML risk subject is a subject who has an anti-JCV antibody index level (e.g., a mean index level) of less than or equal to 0.9. Anti-JC virus index values are calculated from a two-step ELISA antibody assay of serum/plasma (STRATIFY JCV™Antibody (with Index) with Reflex to Inhibition Assay; see, e.g., Lee, P. et al. J of Clin Virol, 2013; 57(2):141-146, incorporated herein by reference). Antibody index level, assays for assessing index level, and the use of such index levels and assays, for determining PML risk are described in, e.g., WO 2012/166971 and WO 2014/193804.

A subject may be considered a high PML risk if the subject tested seropositive for anti-JCV antibodies prior to commencement of natalizumab therapy, or if the subject switches from a seronegative anti-JCV antibody status to a seropositive anti-JCV antibody status during natalizumab therapy. In some embodiments, a subject is considered a high PML risk if the subject has an anti-JCV antibody index level of greater than 1.5 prior to commencement of natalizumab therapy, or if the subject switches from a lower anti-JCV antibody index level of less than or equal to 0.9 to a higher anti-JCV antibody index level of greater than 1.5 during natalizumab therapy. For example, prior to starting natalizumab therapy, a subject may be tested for the presence or absence of anti-JCV antibodies. If the test results indicate that the subject is a low PML risk subject (seronegative for anti-JCV antibodies, or having an anti-JCV antibody index level of less than or equal to 0.9), then the subject may be identified as a subject for natalizumab therapy on a SID schedule of 4-week intervals. During the course of the natalizumab therapy on a SID schedule, the subject may be re-tested for the presence or absence of anti-JCV antibodies (e.g., tested every month or every 2, 3, 4, 5 or 6 months, or every year). If upon re-testing the subject has switched from seronegative to seropositive for anti-JCV antibodies, or from having an anti-JCV antibody index level of less than or equal to 0.9 to having an anti-JCV antibody index level of greater than 1.5, then the subject may be identified as a subject for natalizumab therapy on an EID schedule of at least 5-week intervals.

In some embodiments, a subject undergoes SID natalizumab therapy for at least one year before switching an EID natalizumab therapy. For example, a subject may undergo SID natalizumab therapy for at least 18 months, at least two years, at least 30 months, at least three years, at least 42 months, at least four years, at least 54 months, or at least 5 years before switching to EID natalizumab therapy. In such embodiments, the subject may be a high PML risk subject who had an anti-JCV antibody index level of greater than 1.5 prior to commencement of natalizumab therapy, or the subject may be a high PML risk subject who switched to an anti-JCV antibody index level of greater than 1.5 at some point during SID natalizumab therapy (e.g., within the first, second, third, fourth, or fifth year of SID natalizumab therapy).

Also described herein, are methods of extended interval dosing in a subject having a length of treatment-based risk. For example, a subject may be identified as having a length of treatment-based risk if the subject has been treated with SID natalizumab therapy for at least 12 months, at least 18 months, at least two years, at least 30 months, at least three years, at least 42 months, at least four years, at least 54 months, or at least 5 years, and thereby identified as suitable for one or more of the EID natalizumab therapies described herein, and/or treated with one or more of the EID natalizumab therapies described herein.

A single dose of natalizumab for EID natalizumab therapy is typically 300 mg, and a single dose of natalizumab is typically administered intravenously over the course of one hour.

Although the foregoing EID schedules are described in terms of natalizumab therapy, it is understood that such EID schedules are likely to be suitable for use with other α4-integrin binding antibodies that increase PML risk, and in particular those that bind the same epitope as, or compete for epitope binding with, natalizumab, or those that inhibit lymphocyte trafficking to the brain. In embodiments, such an antibody therapy may include a fixed or variable SID interval and an EID interval that is increased relative to the SID interval, e.g., increased by no more than about 275%. In some embodiments, the EID interval may be increased by at least about 25%. In some embodiments, the EID interval may be increased by at least about 25% and no more than about 275%, at least about 25% and no more than about 250%, at least about 25% and no more than about 200%, or at least about 25% and no more than about 150%.

### EXAMPLES

### Introduction to Examples

To assess an individual's risk of progressive multifocal leukoencephalopathy (PML) during standard interval dosing (SID) and extended interval dosing (EID) of TYSABRI®, a large scale analysis of patient data obtained from the Touch Database was performed, and included all patients with a known positive anti-JCV antibody serostatus and a known status of prior immunosuppressant use. The Touch Database included patient demographics, TYSABRI® dosing information, anti-JCV antibody status, PML status, and history of previous treatment with immunosuppressive therapy. Patients with a history of any interval >12 weeks (“dosing gap”) or <3 weeks (“overdose”) between two consecutive infusions were excluded. Three analyses of patient data were performed with each analysis using a defined dosing definition of SID and EID as shown in Table 1. The three planned analyses and their respective EID and SID inclusion criteria were developed and finalized under conditions blinded to PML events.

Statistical Analysis

Demographic and treatment history data for the overall study population and for each EID analysis cohort were summarized by descriptive statistics. For the three planned analyses, time-to-event (PML occurrence) analyses using Kaplan-Meier estimates of cumulative risk were performed for the EID and SID cohorts. Time-to-event was based on time since initiation of natalizumab treatment. A log-rank test was performed to compare the time-to-event between the EID and SID cohorts. The conditional probability of PML in each exposure epoch (defined as a series of 12 infusions) was derived for the EID and SID cohorts using the life-table method stratified by prior immunosuppressant use (only 5% of patients had prior immunosuppressant use; therefore data are shown for patients without prior use). The PML hazard ratio (HR) in the EID and SID cohorts was estimated using a time-varying covariate Cox regression model adjusted for age, sex, calendar year of the start of natalizumab treatment, and prior immunosuppressant use (yes/no) as covariates and the cumulative number of infusions as the time-varying covariate.

For each analysis, the PML HR estimate (EID vs SID) and its 95% confidence interval (CI) from the Cox model were the primary basis of inference. Specifically, if the HR upper 95% CI limit was <1, the EID cohort would be considered to have a lower risk of PML than the SID cohort. If the HR point estimate was ≥0.9 and ≤1.1, the EID and SID cohorts would be considered to have similar risks. If the HR lower 95% CI limit was >1, the EID cohort would be considered to have greater risk. At the time of analysis plan specification, the anticipated study population sizes and expected number of PML events predicted an approximately 85% power to detect a risk reduction ≥50% (a hazard ratio ≤0.5) as defined by the above rules of inference.

The statistical analysis plan was developed and finalized under conditions blinded to PML events. PML data from the Tysabri Global Safety Database were merged with TOUCH after the analysis plan was finalized.

TABLE 1
Definitions of SID and EID cohorts.

| | Definition Summary | |
|---|---|---|
| Definition* | EID | SID |
| 1 | In the last 18 months** of | In the last 18 months** of |
| | treatment the total | treatment the total |
| | number of infusions <=15 | number of infusions ≥16 |
| 2 | 11 or more infusions in the | 10 or less infusions in the 365 |
| | 365 days prior to | days prior to any |
| | any infusion | infusion |
| 3 | Patients with an average | Patients with an average |
| | number of infusions | number of infusions |
| | per year >10.0 (total | <=10.0 (total number of |
| | number of infusions/total | infusions/total |
| | per year | number of years of treatment |
| | number of years of | <=10.0) |
| | treatment >10.0) | |

*Patients with dosing gap >12 weeks or overdose <3 weeks were excluded in all definitions.
**month defined as 30 days.

### Example 1—Primary Analysis: PML Cases were Reduced in the EID-1 Cohort as Compared to the SID-1 Cohort

The primary analysis assessed PML risk associated with the last 18 months of recorded infusion history. The EID cohort was defined as follows: in the last 18 months (month defined as 30 days) of treatment, the total number of infusions was less than or equal to 15. Patients with the defined dosing pattern in the last 18 months were included in the EID cohort. The SID cohort was defined as follows: in the last 18 months (month defined as 30 days) of treatment, the total number of infusions was greater than or equal to 16. Patients with the defined dosing pattern in the last 18 months were included in the SID cohort. The number of patients who were anti-JCV antibody positive in the EID and SID cohorts are shown in Table 2. Patient demographics are shown in Table 3.

TABLE 2
Number of patients in the EID-1 and SID-1 cohorts.

| | SID group | ED group |
|---|---|---|
| Number of Patients | 40017 | 14868 |
| + Known anti-JCV Ab positive | 18438 | 7543 |
| + Without gap overdose | 13132 | 1988 |

TABLE 3
Patient demographics for patients in the EID-1 and SID-1 cohorts.

| | Primary analysis | | | |
|---|---|---|---|---|
| | EID-1° group | | SID-1° group | |
| Characteristic | (n = 1988) | | (n = 13 132) | |
| Females, n (%)* | 1376 | (69) | 8846 | (67) |
| Age at first infusion, mean (SD), y | 42 · 9 | (11 · 3) | 44 · 0 | (11 · 0) |
| Prior IS therapy, n (%)† | 95 | (5) | 689 | (5) |
| Number of natalizumab infusions, | 50 | (11, 132) | 46 | (17, 142) |
| median (min, max) | | | | |
| Duration of natalizumab treatment, | 59 | (19, 130) | 44 | (19, 131) |
| median (min, max), months | | | | |
| ADI, days | | | | |
| Mean (SD) | 36 · 7 | (4 · 9) | 30 · 0 | (1 · 6) |

| | | |
|---|---|---|
| Q1, Q3 | 33, 39 | 29, 31 |
| Ever anti-JCV antibody positive | 3331 | 13132 |

*Analysis population includes the patients who were anti-JCV antibody positive and had neither treatment gap nor overdosing. Numbers in parentheses are percentages.

The total number of TYSABRI® infusions (Table 4) and the total duration of TYSABRI® treatment (Table 5) for the SID and EID cohorts was determined. The number of TYSABRI® infusions before (Table 6) and on/after (Table 7) the start of the EID/SID treatment regimen was also determined for the SID and EID cohorts.

TABLE 4
Total number of TYSABRI ® infusions for patients
in the EID-1 and SID-1 cohorts.

| | SID group | EID group |
|---|---|---|
| Number of Patients | 13132 | 1988 |
| Total Number of | | |
| TYSABRI ® infusions | | |
| <1 | 0 | 0 |
| 1-12 | 0 | 0 (<1) |
| 13-24 | 2185 (17) | 321 (16) |
| 25-36 | 2893 (22) | 326 (16) |
| 37-48 | 1905 (15) | 310 (16) |
| 49-60 | 1710 (13) | 289 (15) |
| 61-72 | 1449 (11) | 196 (10) |
| >=73 | 2290 (23) | 542 (27) |
| n | 13132 | 1988 |
| Mean | 52.7 | 55.0 |
| SD | 28.94 | 28.92 |
| Median | 46.0 | 50.0 |
| Min, Max | 17, 142 | 11, 132 |

*Analysis population includes the patients who were anti-JCV antibody positive and had neither treatment gap nor overdosing.
Numbers in parentheses are percentages.

TABLE 5
Total duration of TYSABRI ® treatment for
patients in the EID-1 and SID-1 cohorts.

| | SID group | EID group |
|---|---|---|
| Total Duration of | | |
| TYSABRI ® Treatment | | |
| (Months) | | |
| <1 | 0 | 0 |
| 1-12 | 0 | 0 |
| 13-24 | 2536 (19) | 171 (9) |
| 25-36 | 2747 (21) | 315 (16) |
| 37-48 | 1864 (14) | 277 (14) |
| 49-60 | 1754 (13) | 268 (13) |
| 61-72 | 1456 (11) | 257 (13) |
| >=73 | 2775 (21) | 700 (35) |
| n | 13132 | 3331 |
| Mean | 51.3 | 63.6 |
| SD | 28.02 | 31.11 |
| Median | 44.0 | 59.0 |
| Min, Max | 19, 131 | 19, 130 |

*Analysis population includes the patients who were anti-JCV antibody positive and had neither treatment gap nor overdosing.
Numbers in parentheses are percentages.

TABLE 6
Number of TYSABRI ® infusions before last 18 months
for patients in the EID-1 and SID-1 cohorts.

| | SID group | EID group |
|---|---|---|
| Number of TYSABRI ® | | |
| infusions before last | | |
| 18 months | | |
| <1 | 19 (<1) | 0 |
| 1-12 | 4059 (31) | 354 (18) |
| 13-24 | 2135 (16) | 326 (16) |
| 25-36 | 1737 (13) | 299 (15) |
| 37-48 | 1637 (12) | 292 (15) |
| 49-60 | 1198 (9) | 184 (9) |
| 61-72 | 761 (6) | 180 (9) |
| >=73 | 1586 (12) | 353 (18) |
| n | 13132 | 1988 |
| Mean | 34.0 | 42.0 |
| SD | 28.84 | 29.05 |
| Median | 27.0 | 37.0 |
| Min, Max | 0, 121 | 1, 117 |

*Analysis population includes the patients who were anti-JCV antibody positive and had neither treatment gap nor overdosing.
Numbers in parentheses are percentages.

TABLE 7
Number of TYSABRI ® Infusions within the last 18
months for patients in the EID-1 and SID-1 cohorts.

| | SID group | EID group |
|---|---|---|
| Number of | | |
| TYSABRI ® infusions | | |
| within last 18 months | | |
| <1 | 0 | 0 |
| 1-12 | 0 | 635 (32) |
| 13-24 | 13131 (>99) | 1353 (68) 20 |
| 25-36 | 1 (<1) | 0 |
| 37-48 | 0 | 0 |
| 49-60 | 0 | 0 |
| 61-72 | 0 | 0 |
| >=73 | 0 | 0 |
| n | 13132 | 1988 |
| Mean | 18.7 | 13.0 |
| SD | 1.20 | 1.83 25 |
| Median | 19.0 | 13.0 |
| Min, Max | 16, 25 | 8, 15 |

*Analysis population includes the patients who were anti-JCV antibody positive and had neither treatment gap nor overdosing.
Numbers in parentheses are percentages.

A time to event (PML occurrence) analysis using Kaplan-Meier estimate of cumulative risk was performed for EID and SID cohorts, stratified by prior use of immunosuppressants. Within each stratum of prior use of immunosuppressants (yes/no), a log-rank test was performed to compare the time to event between the EID and SID cohorts.

The PML risk estimates per 1,000 patients were consistently lower in the EID cohort as compared to the SID cohort over the course of treatment with TYSABRI® for both patients having prior treatment with an immunosuppressant and those without such prior treatment (Table 8). Table 8 shows a table of PML risk estimates per 1,000 anti-JCV antibody positive patients grouped according to dosing schedules described in this Example. As described above, the extended interval dosing (EID) cohort was defined as patients having less than or equal to 15 total number of infusions within the last 18 months (month defined as 30 days). The standard interval dosing (SID) cohort was defined as patients having greater than or equal to 16 total number of infusions within the last 18 months (month defined as 30 days). PML risk estimates were obtained for patients previously treated with an immunosuppressant (Prior immunosuppression (IS)) and patients without prior immunosuppressant treatment (No Prior IS).

TABLE 8
PML Risk Estimates per 1,000 Patients (with 95% Cis) using Life Table
Method EID definition 1a-Analysis Population

| | PML Incidence per 1,000 Patients (95% CI) Anti-JCV Antibody Positive | | | |
|---|---|---|---|---|
| Tysabri | Prior IS | | No Prior IS | |
| Exposure | SID group | EID group | SID group | EID group |
| 1-12 | 0.00 (0.00-5.34) | 0.00 (0.00-38.09) | 0.00 (0.00-0.31) | 0.00 (0.00-2.04) |
| 13-24 | 0.00 (0.00-5.80) | 0.00 (0.00-41.05) | 0.28 (0.06-0.80) | 0.00 (0.00-2.22) |
| 25-36 | 1.97 (0.05-10.94) | 0.00 (0.00-49.28) | 0.46 (0.13-1.19) | 0.00 (0.00-2.70) |
| 37-48 | 5.24 (0.63-18.78) | 0.00 (0.00-60.61) | 2.02 (1.08-3.45) | 0.00 (0.00-3.41) |
| 49-60 | 17.61 (5.74-40.61) | 0.00 (0.00-78.17) | 3.96 (2.38-6.17) | 1.23 (0.03-6.86) |
| 61-72 | 9.71 (1.18-34.63) | 28.57 (0.72-149.2) | 4.46 (2.50-7.35) | 1.70 (0.04-9.42) |
| >=73 | 44.94 (12.38-111.1) | 0.00 (0.00-218.0) | 14.70 (9.00-22.60) | 0.00 (0.00-14.71) |

NOTE:
Analysis population includes the patients who have anti-JCV antibody positive and have neither treatment gap nor over dosing.

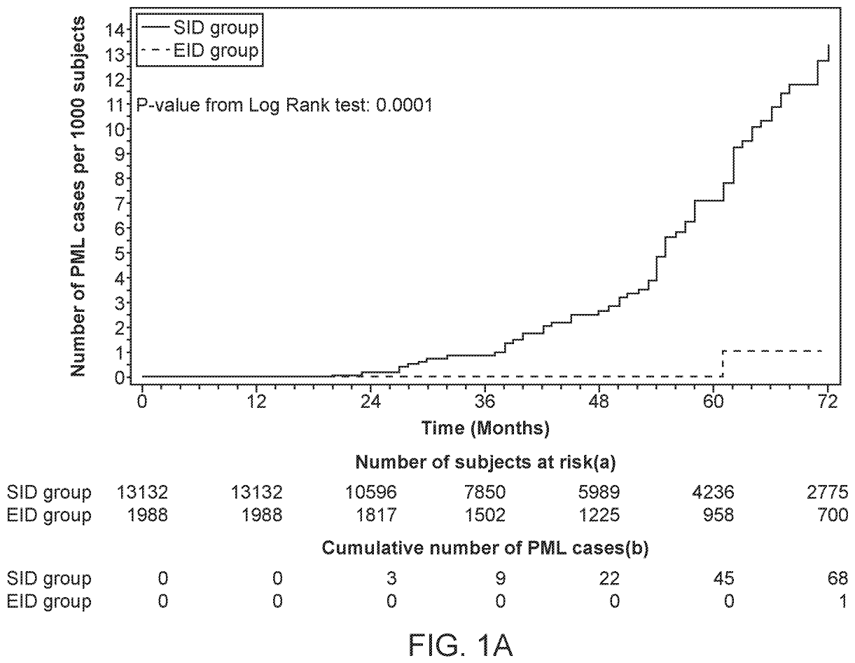

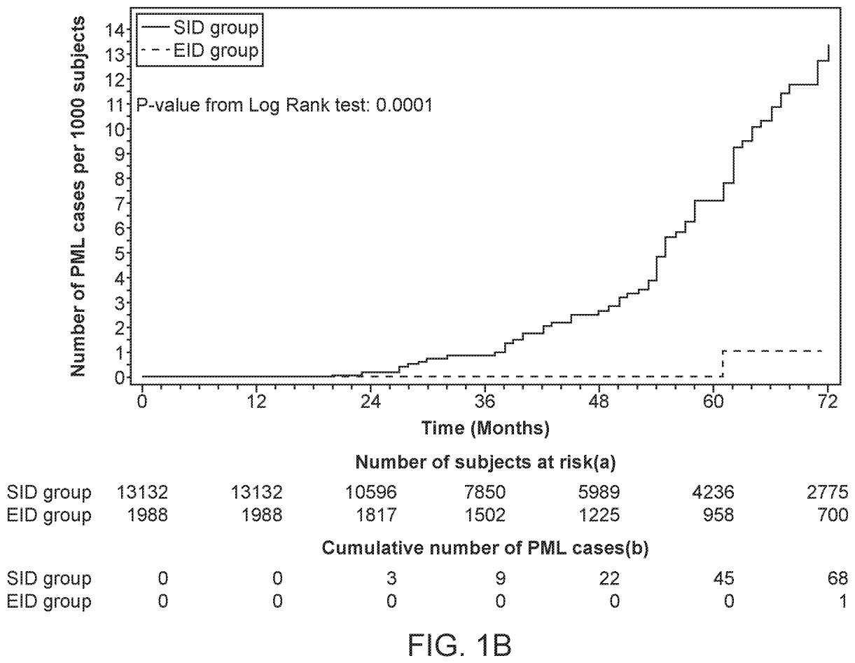

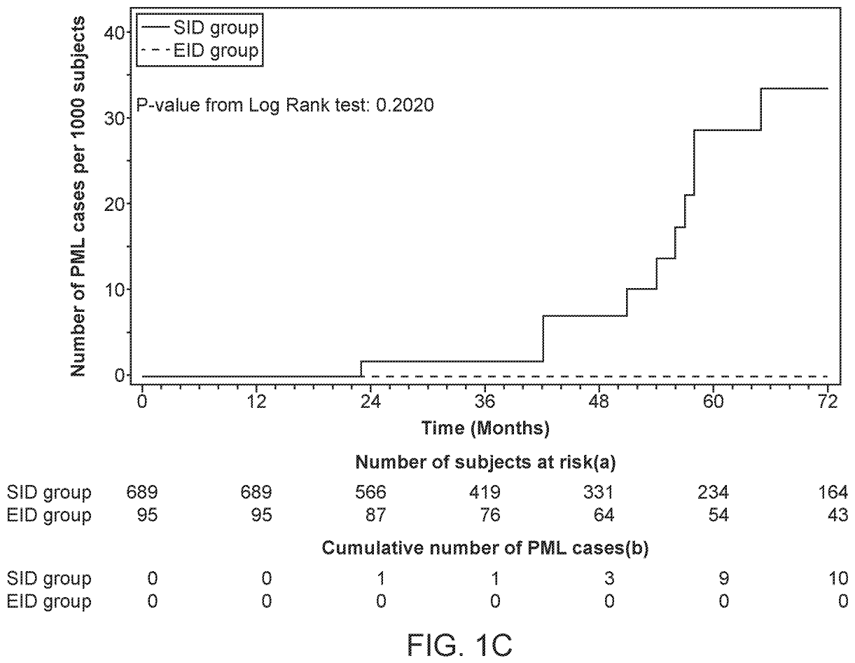

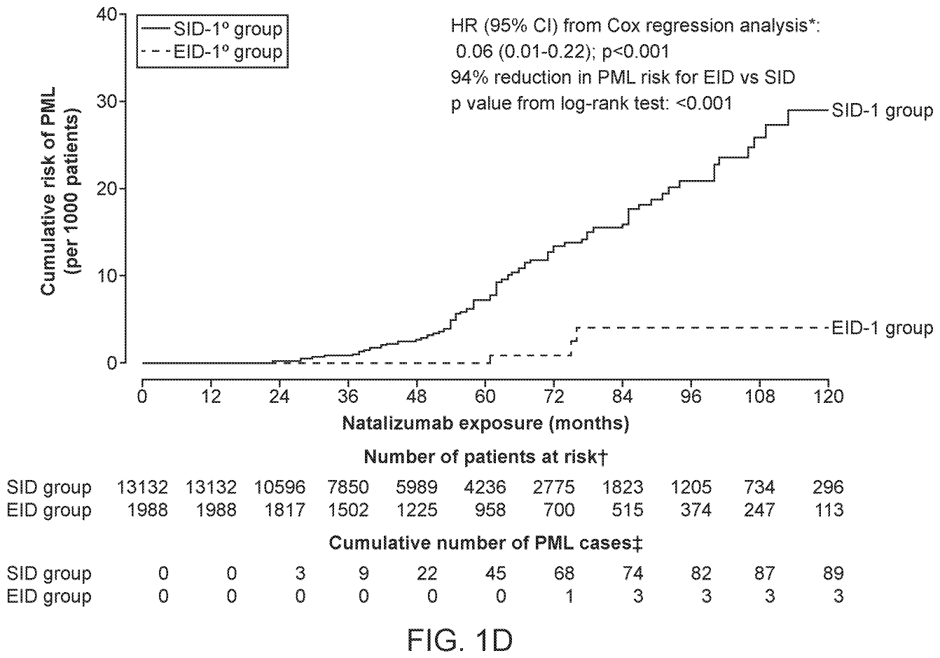

The number of PML cases per patient was significantly reduced in the EID cohort as compared to the SID cohort over 72 months (FIG. 1A) and 120 months (FIG. 1D). The EID cohort had significantly reduced numbers of PML cases per patient as compared to the SID cohort for patients without prior immunosuppressant treatment (FIG. 1B) and with previous immunosuppressant treatment (FIG. 1C).

These results demonstrated that the number of PML cases was significantly reduced in patients treated with the EID regimen as compared to those treated with the SID regimen when the EID regimen was defined as 15 or less infusions in the last 18 months and the SID regimen was defined as 16 or more infusions in the last 18 months.

### Example 2—Secondary Analysis: PML Cases were Reduced in the EID-2 Cohort as Compared to the SID-2 Cohort

The secondary analysis assessed the effect of any prolonged period of EID in the patient's infusion history on PML risk. The EID cohort was defined as follows: in the last 365 days of treatment, the total number of infusions was 10 or less. Patients with the defined dosing pattern in the last 365 days were included in the EID cohort and received consecutive EID infusions for ≥6 months. The SID cohort was defined as follows: in the last 365 days of treatment, the total number of infusions was 11 or more. Patients with the defined dosing pattern in the last 365 days were included in the SID cohort and received consecutive SID infusions for ≥6 months. The number of patients who were anti-JCV antibody positive in the EID and SID cohorts are shown in Table 9. Patient demographics are shown in Table 10.

TABLE 9
Number of patients in the EID-2 and SID-2 cohorts.

| | SID group | EID group |
|---|---|---|
| Number of Patients | 42979 | 26118 |
| +Known anti-JCV Ab positive | 18134 | 13067 |
| +Without gap overdose | 16386 | 3764 |
| +Patients with one sequence | 15424 | 3331 |

TABLE 10
Patient demographics for patients in the EID-2 and SID-2 cohorts.

| | Secondary analysis | |
|---|---|---|
| | EID-2° group | SID-2° group |
| Characteristic | (n = 3331) | (n = 15 424) |
| Females, n (%)* | 2293 (69) | 10 239 (66) |
| Age at first infusion, mean (SD), y | 43.0 (11.2) | 43.9 (11.4) |
| Prior IS therapy, n (%)† | 175 (5) | 799 (5) |
| Number of natalizumab infusions, | 51 (6, 137) | 27 (7, 142) |
| median (min, max) | | |
| Duration of natalizumab treatment, | 56 (8, 131) | 26 (7, 130) |
| median (min, max), months | | |
| ADI, days | | |
| Mean (SD) | 35.0 (4.9) | 29.8 (1.7) |
| Q1, Q3 | 32, 37 | 29, 31 |
| Ever anti-JCV antibody positive | 3331 | 15424 |

*Analysis population includes the patients who were anti-JCV antibody positive and had neither treatment gap nor overdosing.
Numbers in parentheses are percentages.

The total number of TYSABRI® infusions (Table 11) and the total duration of TYSABRI® treatment (Table 12) for the SID and EID cohorts was determined. The number of TYSABRI® infusions before (Table 13) and on/after (Table 14) the start of the EID/SID treatment regimen was also determined for the SID and EID cohorts.

TABLE 11
Total Number of TYSABRI ® Infusions patients
in the EID-2 and SID-2 cohorts.

| | SID group | EID group |
|---|---|---|
| Number of Patients | 15424 | 3331 |
| Total Number of | | |
| TYSABRI ® infusions | | |
| <1 | 0 | 0 |
| 1-12 | 1995 (13) | 116 ( 3) |
| 13-24 | 4949 (32) | 457 (14) |
| 25-36 | 2601 (17) | 486 (15) |
| 37-48 | 1530 (10) | 501 (15) |
| 49-60 | 1252 (8) | 518 (16) |
| 61-72 | 1071 (7) | 360 (11) |
| >=73 | 2026 (13) | 893 (27) |
| n | 15424 | 3331 |
| Mean | 38.0 | 55.3 |
| SD | 28.57 | 29.94 |
| Median | 27.0 | 51.0 |
| Min, Max | 7, 142 | 6, 137 |

*Analysis population includes the patients who were anti-JCV antibody positive and had neither treatment gap nor overdosing.
Numbers in parentheses are percentages.

TABLE 12
Total Duration of TYSABRI ® Treatment patients
in the EID-2 and SID-2 cohorts.

| | SID group | EID group |
|---|---|---|
| Total Duration of | | |
| TYSABRI ® Treatment | | |
| (Months) | | |
| <1 | 0 | 0 |
| 1-12 | 2320 (15) | 46 (1) |
| 13-24 | 5021 (33) | 336 (10) |
| 25-36 | 2455 (16) | 486 (15) |
| 37-48 | 1498 (10) | 473 (14) |
| 49-60 | 1303 (8) | 498 (15) |
| 61-72 | 1071 (7) | 429 (13) |
| >=73 | 1756 (11) | 1063 (32) |
| n | 15424 | 3331 |
| Mean | 36.4 | 60.7 |
| SD | 27.11 | 30.78 |
| Median | 26.0 | 56.0 |
| Min, Max | 7, 130 | 8, 131 |

*Analysis population includes the patients who were anti-JCV antibody positive and had neither treatment gap nor overdosing.
Numbers in parentheses are percentages.

TABLE 13
Number of TYSABRI ® Infusions before start of EID/SID
regimen patients in the EID-2 and SID-2 cohorts.

| | SID group | EID group |
|---|---|---|
| Number of TYSABRI ® | | |
| infusions before EID/SID | | |
| regimen start | | |
| <1 | 0 | 0 |
| 1-12 | 15328 (>99) | 830 (25) |
| 13-24 | 94 (<1) | 801 (24) |
| 25-36 | 2 (<1) | 602 (18) |
| 37-48 | 0 | 369 (11) |
| 49-60 | 0 | 255 (8) |
| 61-72 | 0 | 180 (5) |
| >=73 | 0 | 294 (9) |
| n | 15424 | 3331 |
| Mean | 1.3 | 32.0 |
| SD | 1.40 | 25.28 |
| Median | 1.0 | 25.0 |
| Min, Max | 1, 31 | 1, 121 |

*Analysis population includes the patients who were anti-JCV antibody positive and had neither treatment gap nor overdosing.
Numbers in parentheses are percentages.

TABLE 14
Number of TYSABRI ® Infusions on/after start of
EID/SID regimen patients in the EID-2 and SID-2 cohorts.

| | SID group | EID group |
|---|---|---|
| Number of TYSABRI ® infusions | | |
| on/after EID/SID regimen start | | |
| <1 | 0 | 0 |
| 1-12 | 2646 (17) | 1213 (36) |
| 13-24 | 4746 (31) | 987 (30) |
| 25-36 | 2340 (15) | 504 (15) |
| 37-48 | 1470 (10) | 295 (9) |
| 49-60 | 1249 (8) | 137 (4) |
| 61-72 | 1049 (7) | 98 (3) |
| >=73 | 1924 (12) | 97 (3) |
| n | 15424 | 3331 |
| Mean | 36.8 | 23.3 |
| SD | 28.55 | 18.79 |
| Median | 25.0 | 17.0 |
| Min, Max | 6, 141 | 4, 118 |

*Analysis population includes the patients who were anti-JCV antibody positive and had neither treatment gap nor overdosing.
Numbers in parentheses are percentages.

A time to event (PML occurrence) analysis using Kaplan-Meier estimate of cumulative risk was performed for EID and SID cohorts, stratified by prior use of immunosupressants. Within each stratum of prior use of immunosuppressants (yes/no), a log-rank test was performed to compare the time to event between the EID and SID cohorts.

The PML risk estimates per 1,000 patients were consistently lower in the EID cohort as compared to the SID cohort over the course of treatment with TYSABRI® for both patients having prior treatment with an immunosuppressant and those without such prior treatment (Table 15). As described above, Table 15 shows a table of PML risk estimates per 1,000 anti-JCV antibody positive patients grouped according to dosing schedule described in this Example. The SID cohort was defined as patients having 11 or more infusions within the last 365 days. The EID cohort was defined as patients having 10 or less infusions within the last 365 days. PML risk estimates were obtained for patients previously treated with an immunosuppressant (Prior IS) and patients without prior immunosuppressant treatment (No Prior IS).

TABLE 15
PML Risk Estimates per 1,000 Patients (with 95% Cis) using Life Table
Method EID definition 2a-Analysis Population

| | PML Incidence per 1,000 Patients (95% CI) Anti-JCV Antibody Positive | | | |
|---|---|---|---|---|
| Tysabri | Prior IS | | No Prior IS | |
| Exposure | SID group | EID group | SID group | EID group |
| 1-12 | 0.00 (0.00-4.93) | 0.00 (0.00-21.34) | 0.00 (0.00-0.28) | 0.00 (0.00-1.24) |
| 13-24 | 0.00 (0.00-6.55) | 0.00 (0.00-23.52) | 0.28 (0.22-1.32) | 0.00 (0.00-1.35) |
| 25-36 | 2.73 (0.07-15.13) | 0.00 (0.00-27.35) | 0.46 (0.09-1.35) | 0.44 (0.01-2.43) |
| 37-48 | 3.83 (0.10-21.16) | 9.01 (0.23-49.17) | 2.58 (1.33-4.50) | 0.00 (0.00-2.00) |
| 49-60 | 26.04 (8.51-59.72) | 0.00 (0.00-43.47) | 4.14 (2.26-6.93) | 1.45 (0.18-5.23) |
| 61-72 | 14.71 (1.79-52.11) | 16.39 (0.41-87.99) | 4.74 (2.37-8.46) | 2.04 (0.25-7.35) |
| >=73 | 35.71 (4.35-123.1) | 0.00 (0.00-127.7) | 14.07 (7.51-23.94) | 12.20 (3.97-28.23) |

NOTE:
Analysis population includes the patients who have anti-JCV antibody positive and have neither treatment gap nor over dosing and only one EID/SID sequence.
Numbers in parentheses are percentages.

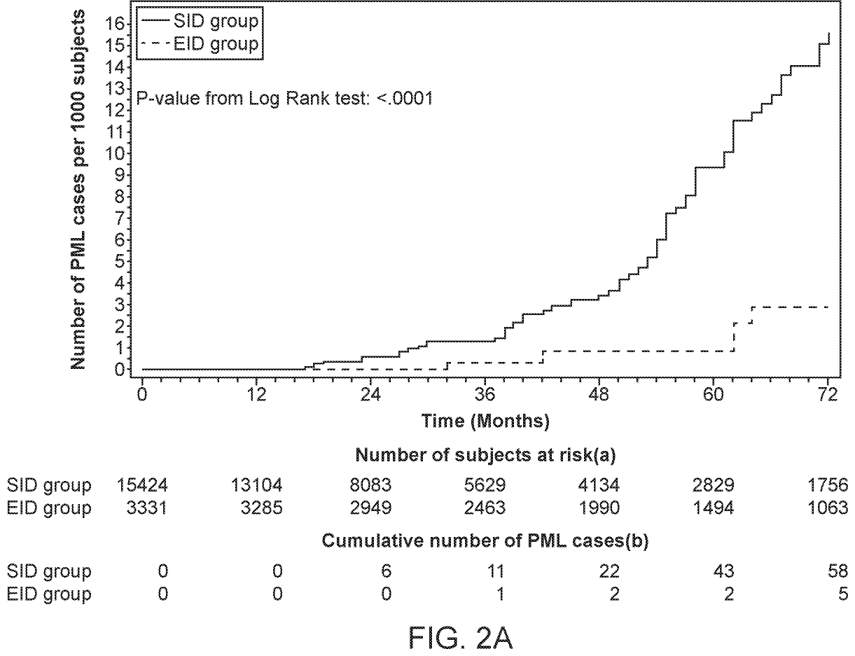

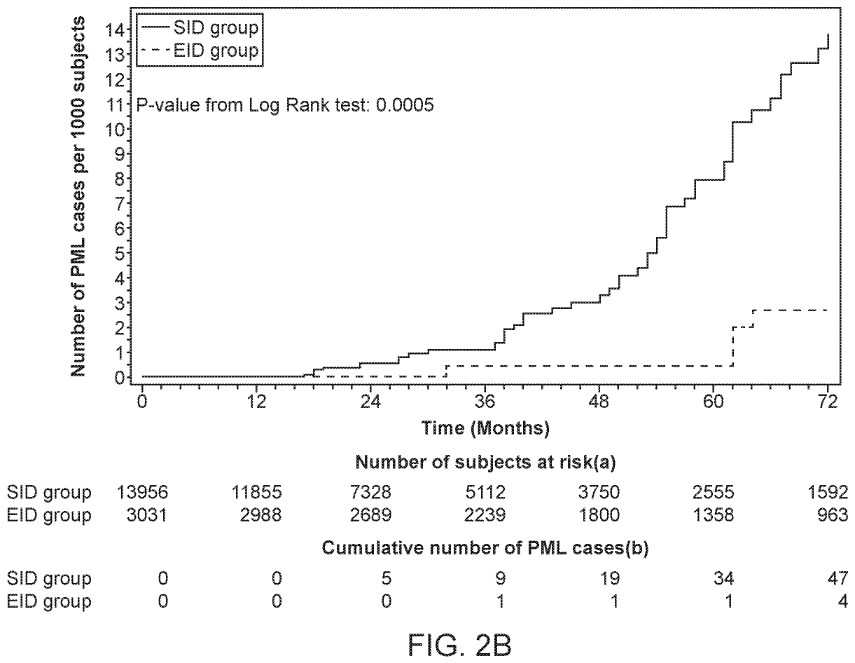

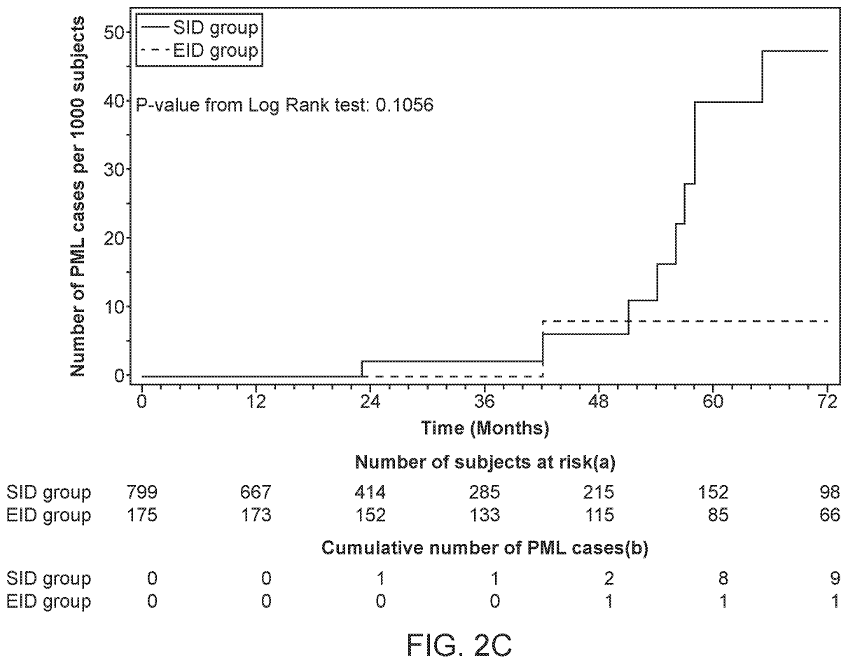

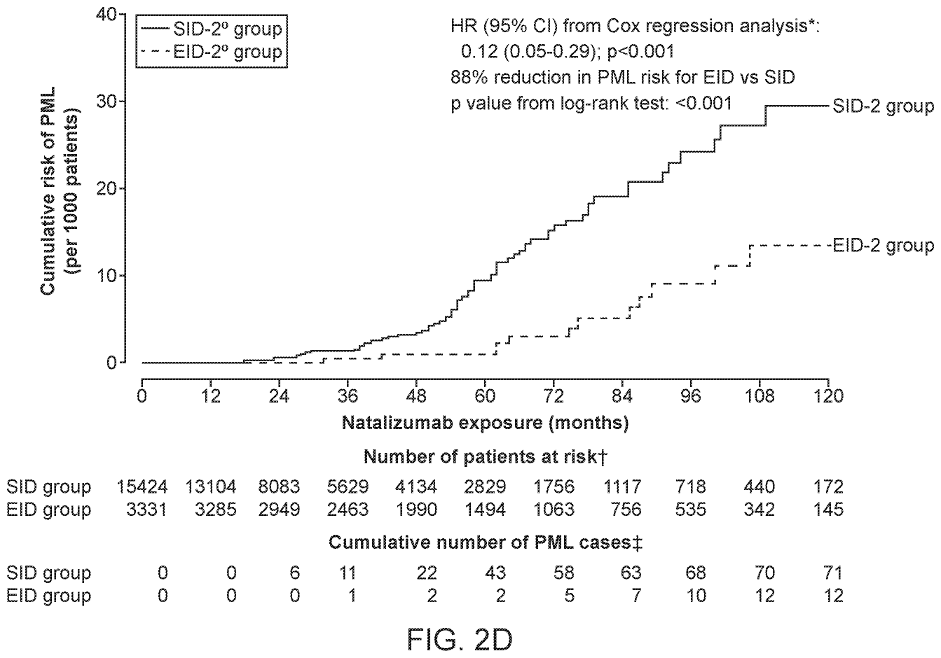

The number of PML cases per patient was significantly reduced in the EID cohort as compared to the SID cohort over 72 months (FIG. 2A) and 120 months (FIG. 2D). The EID cohort had significantly reduced numbers of PML cases per patient as compared to the SID cohort for patients without prior immunosuppressant treatment (FIG. 2B) and with previous immunosuppressant treatment (FIG. 2C).

These results demonstrated that the number of PML cases was significantly reduced in patients treated with the EID regimen as compared to those treated with the SID regimen when the EID regimen was defined as 10 or less infusions in the 365 day period immediately prior to a treatment dose and the SID regimen was defined as 11 or more infusions in the 365 day period immediately prior to a treatment dose.

### Example 3—Tertiary Analysis: PML Cases were Reduced in the EID-3 Cohort as Compared to the SID-3 Cohort

The tertiary analysis assessed the effect of a dosing history consisting primarily of EID on PML risk. The EID cohort was defined as follows: the average number of infusions per year was less than or equal to 10 over the entire treatment duration. Patients with the defined dosing pattern over the entire treatment duration were included in the EID cohort. The SID cohort was defined as follows: the average number of infusions per year was greater than 10 over the entire treatment duration. Patients with the defined dosing pattern over the entire treatment duration were included in the SID cohort. The number of patients who were anti-JCV antibody positive in the EID and SID cohorts are shown in Table 16. Patient demographics are shown in Table 17.

TABLE 16
Number of patients in the EID-3 and SID-3 cohorts.

| | SID group | EID group |
|---|---|---|
| Number of Patients | 75699 | 14337 |
| +Known anti-JCV Ab positive | 28861 | 6660 |
| +Without gap overdose | 23168 | 815 |

TABLE 17
Patient demographics for patients in the EID-3 and SID-3 cohorts.

| | Tertiary analysis | |
|---|---|---|
| | EID-3° group | SID-3° group |
| Characteristic | (n = 815) | (n = 23 168) |
| Females, n (%)* | 539 (66) | 15 636 (67) |
| Age at first infusion, mean (SD), y | 42.0 (11.4) | 43.9 (11.6) |
| Prior IS therapy, n (%)† | 49 (6) | 1310 (6) |
| Number of natalizumab infusions, | 32 (2, 103) | 26 (1, 142) |
| median (min, max) | | |
| Duration of natalizumab treatment, | 43 (3, 129) | 25 (1, 131) |
| median (min, max), months | | |
| ADI, days | | |
| Mean (SD) | 43.0 (5.4) | 30.5 (2.6) |
| Q1, Q3 | 39, 45 | 29, 31 |
| Ever anti-JCV antibody positive | 3331 | 23168 |

*Analysis population includes the patients who were anti-JCV antibody positive and had neither treatment gap nor overdosing.
Numbers in parentheses are percentages.

The total number of TYSABRI® infusions (Table 18) and the total duration of TYSABRI® treatment (Table 19) for the SID and EID cohorts was determined.

TABLE 18
Total Number of TYSABRI ® Infusions
patients in the EID-3 and SID-3 cohorts.

| | SID group | EID group |
|---|---|---|
| Number of | 23168 | 815 |
| Patients | | |
| Total Number of | | |
| TYSABRI ® infusions | | |
| <1 | 0 | 0 |
| 1-12 | 5680 (25) | 145 (18) |
| 13-24 | 5357 (23) | 191 (23) |
| 25-36 | 3103 (13) | 116 (14) |
| 37-48 | 2089 (9) | 126 (15) |
| 49-60 | 1905 (8) | 94 (12) |
| 61-72 | 1593 (7) | 52 (6) |
| >=73 | 3441 (15) | 91 (11) |
| n | 23168 | 815 |
| Mean | 37.1 | 36.8 |
| SD | 31.36 | 24.48 |
| Median | 26.0 | 32.0 |
| Min, Max | 1, 142 | 2, 103 |

*Analysis population includes the patients who were anti-JCV antibody positive and had neither treatment gap nor overdosing. Numbers in parentheses are percentages.

TABLE 19
Total Duration of TYSABRI ® Treatment
patients in the EID-3 and SID-3 cohorts.

| | SID group | EID group |
|---|---|---|
| Total Duration of | | |
| TYSABRI ® Treatment | | |
| (Months) | | |
| <1 | 0 | 0 |
| 1-12 | 5981 (26) | 66 (8) |
| 13-24 | 5351 (23) | 172 (21) |
| 25-36 | 2939 (13) | 123 (15) |
| 37-48 | 2057 (9) | 84 (10) |
| 49-60 | 1926 (8) | 96 (12) |
| 61-72 | 1637 (7) | 76 (9) |
| >=73 | 3277 (14) | 198 (24) |
| n | 23168 | 815 |
| Mean | 36.4 | 49.6 |
| SD | 30.98 | 32.56 |
| Median | 25.0 | 43.0 |
| Min, Max | 1, 131 | 3, 129 |

*Analysis population includes the patients who were anti-JCV antibody positive and had neither treatment gap nor overdosing. Numbers in parentheses are percentages.

A time to event (PML occurrence) analysis using Kaplan-Meier estimate of cumulative risk was performed for EID and SID cohorts, stratified by prior use of immunosupressants. Within each stratum of prior use of immunosuppressants (yes/no), a log-rank test was performed to compare the time to event between the EID and SID cohorts.

The PML risk estimates per 1,000 patients were consistently lower in the EID cohort as compared to the SID cohort over the course of treatment with TYSABRI® for both patients having prior treatment with an immunosuppressant and those without such prior treatment (Table 20). As described above, Table 20 shows a table of PML risk estimates per 1,000 anti-JCV antibody positive patients grouped according to dosing schedule described in this Example. The SID cohort was defined as patients averaging less than or equal to 10 infusions per year. The EID cohort was defined as patients averaging greater than or equal to 10 infusions per year. PML risk estimates were obtained for patients previously treated with an immunosuppressant (Prior IS) and patients without prior immunosuppressant treatment (No Prior IS)

TABLE 20
PML Risk Estimates per 1,000 Patients (with 95% CIs) using Life Table
Method EID definition 3a-Analysis Population

| | PML Incidence per 1,000 Patients (95% CI) Anti-JCV Antibody Positive | | | |
|---|---|---|---|---|
| Tysabri | Prior IS | | No Prior IS | |
| Exposure | SID group | EID group | SID group | EID group |
| 1-12 | 0.00 (0.00-3.32) | 0.00 (0.00-78.71) | 0.00 (0.00-0.20) | 0.00 (0.00-5.56) |
| 13-24 | 0.00 (0.00-4.79) | 0.00 (0.00-94.89) | 0.52 (0.21-1.07) | 0.00 (0.00-7.21) |
| 25-36 | 1.81 (0.05-10.07) | 0.00 (0.00-115.7) | 0.42 (0.11-1.07) | 0.44 (0.00-9.89) |
| 37-48 | 4.76 (0.58-17.09) | 0.00 (0.00-161.1) | 1.79 (0.95-3.06) | 0.00 (0.00-13.82) |
| 49-60 | 15.77 (5.14-36.42) | 0.00 (0.00-247.1) | 3.67 (2.25-5.67) | 1.45 (0.18-5.23) |
| 61-72 | 12.88 (2.66-37.16) | 0.00 (0.00-369.4) | 4.16 (2.38-6.74) | 2.04 (0.25-7.35) |
| >=73 | 39.60 (10.89-98.31) | 0.00 (0.00-602.4) | 12.75 (7.80-19.62) | 0.00 (0.00-86.04) |

NOTE:
Analysis population includes the patients who have anti-JCV antibody positive and have neither treatment gap nor over dosing.

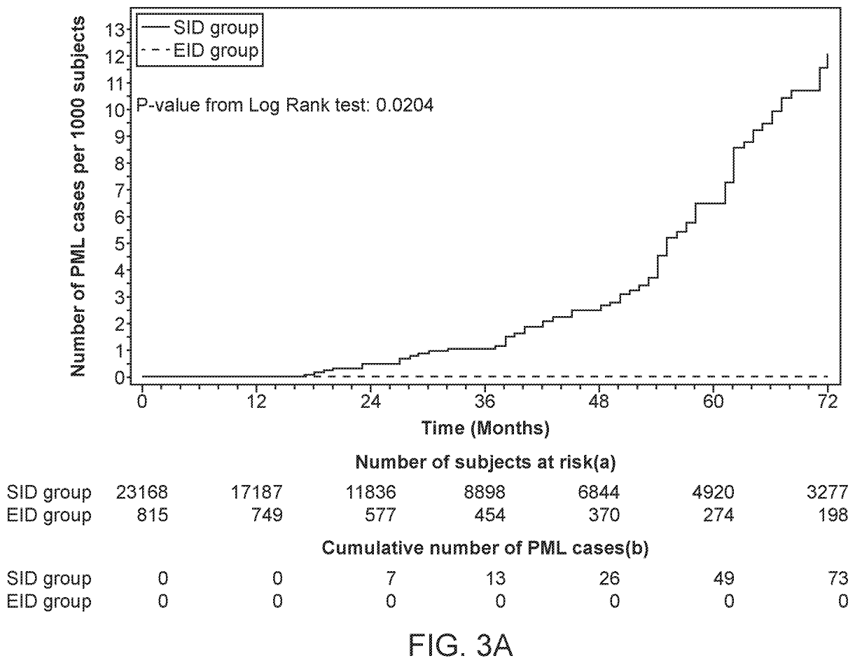

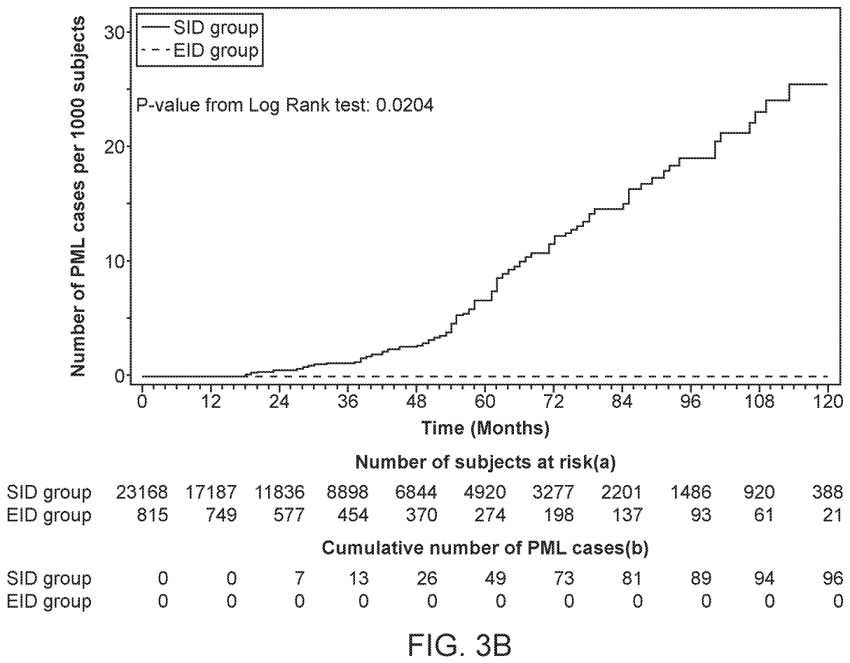

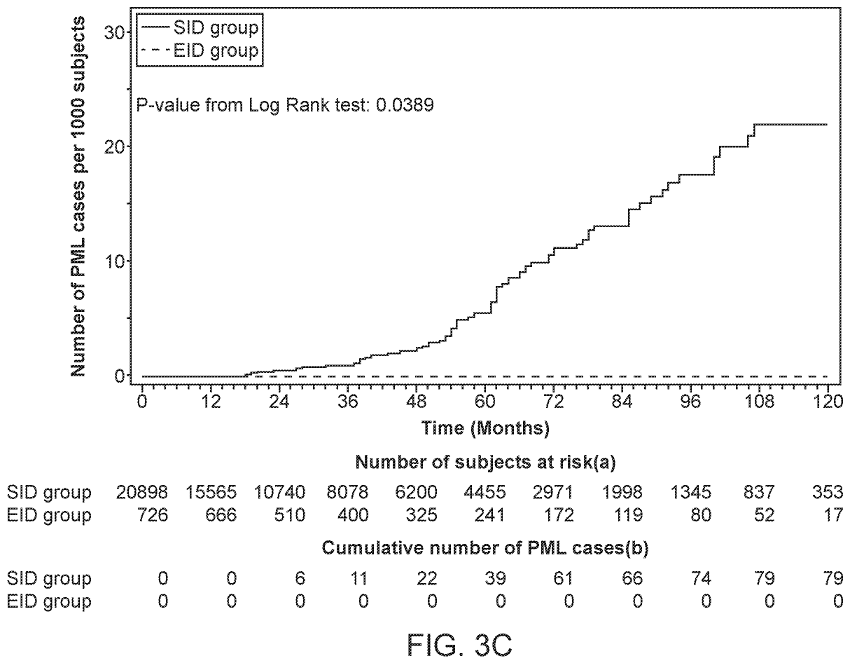

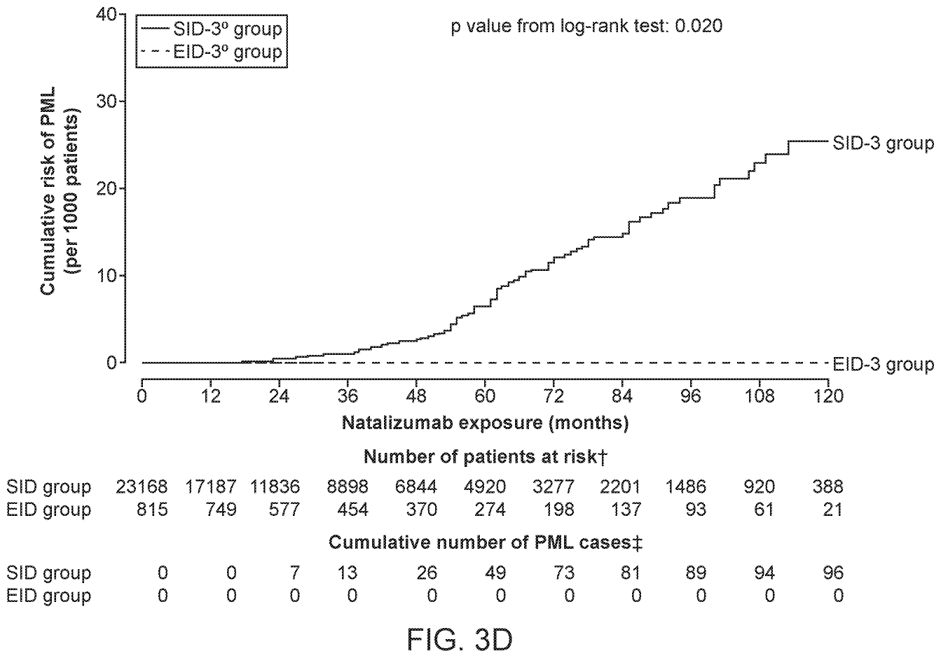

The number of PML cases per patient was significantly reduced in the EID cohort as compared to the SID cohort over 72 months (FIG. 3A) and 120 months (FIG. 3B). The EID cohort had significantly reduced numbers of PML cases per patient as compared to the SID cohort for patients without prior immunosuppressant treatment (FIG. 3C) and with previous immunosuppressant treatment (FIG. 3D).

These results demonstrated that the number of PML cases was significantly reduced in patients treated with the EID regimen as compared to those treated with the SID regimen when the EID regimen was defined as 10 or less infusions in the 365 day period immediately prior to a treatment dose and the SID regimen was defined as 11 or more infusions in the 365 day period immediately prior to a treatment dose.

### Summary of Results for Primary, Secondary, and Tertiary Analyses (Examples 1-3)

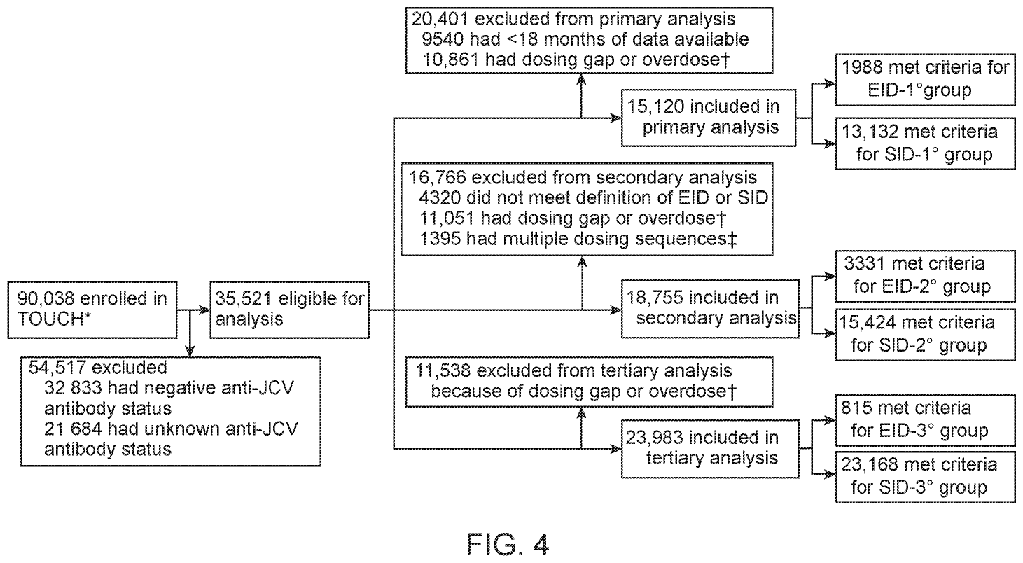

Of the 90,038 patients enrolled in this study, 35,521 were anti-JCV antibody positive and eligible for this study (FIG. 4). After applying the prespecified EID and SID inclusion criteria, the study populations included 1,988 EID and 13,132 SID patients in the primary analysis, 3,331 EID and 15,424 SID patients in the secondary analysis, and 815 EID and 23,168 SID patients in the tertiary analysis. The most common reasons for patient exclusion were the presence of dosing gaps or overdoses in treatment history (criteria applied to primary, secondary, and tertiary analyses) and <18 months of available dosing data (primary analysis only).

The baseline demographics in the EID and SID groups were well balanced across the three analyses (Tables 3, 10, and 17). In all three analyses, EID patients had more natalizumab infusions and longer total duration of natalizumab treatment than SID patients. EID patients included in the primary analysis had received a median (range) of 37 (1-117) infusions before starting EID. In the secondary analysis (in which each infusion was defined as either EID or SID), EID-2° patients had received a median (range) of 25 (1-121) infusions before starting EID. For all three analyses, the average dosing interval (ADI) over the entire treatment duration was 35.0-43.0 days for EID patients and 29.8-30.5 days for SID patients.

The Kaplan-Meier estimated cumulative risk of PML was significantly lower with EID than with SID (FIGS. 1D, 2D and 3D). In the primary and secondary analyses, cumulative risk appeared to separate after 24-36 months, with increasing separation at later time points. Cox regression analysis also identified significant reductions in PML risk with EID treatment in the primary and secondary analyses (both p<0.001; Table 21). The covariate-adjusted HR in the primary analysis was 0.06 (95% CI, 0.01-0.22), corresponding to a relative risk reduction of 94% in EID-1° patients vs SID-1° patients. In the secondary analysis, the covariate-adjusted HR was 0.12 (95% CI, 0.05-0.29), corresponding to a relative risk reduction of 88% in EID-2° patients vs SID-2° patients. As no PML cases were observed with EID in the tertiary analysis, the risk-reduction point estimate was 100% and the Cox regression model 95% CT was non-estimable

TABLE 21
Impact of EID vs SID on PML risk in a Cox regression model in the primary
and secondary analyses*

| | Primary analysis | | Secondary analysis | |
|---|---|---|---|---|
| | Hazard ratio | | Hazard ratio | |
| Risk factor | (95% CI) | p value | (95% CI) | p value |
| Age | 1.00 (0.98-1.02) | 0.999 | 0.99 (0.97-1.01) | 0.411 |
| Sex (male, female) | 1.05 (0.58-1.63) | 0.828 | 0.99 (0.63-1.57) | 0.969 |
| Prior IS use (yes, no) | 2.92 (1.67-5.11) | <0.001 | 2.90 (1.60-5.27) | 0.001 |
| Calendar year at the start of treatment | 0.99 (0.88-1.12) | 0.881 | 0.94 (0.83-1.06) | 0.327 |
| Number of cumulative infusions | 0.91 (0.87-0.95) | <0.001 | 0.97 (0.87-0.94) | <0.001 |
| EID group (EID, SID) | 0.06 (0.01-0.22) | <0.001 | 0.12 (0.05-0.29) | <0.001 |

CI = confidence interval.
EID = extended interval dosing.
IS = immunosuppressant.
PML = progressive multifocal leukoencephalopathy.
SID = standard interval dosing.
*Model includes age, sex, prior use of IS, EID/SID group, and calendar year at the start of natalizumab treatment as covariates. Modelling could not be performed in the tertiary analysis because no PML events occurred in the tertiary analysis EID group.

Prior immunosuppressant use significantly increased PML risk. Covariate-adjusted HRs were 2.92 (95% CI, 1.67-5.11; p<0.001) in the primary analysis and 2.90 (95% CI, 1.60-5.27; p=0.001) in the secondary analysis (Table 21). However, the significance of this observation is limited by the small number of patients with immunosuppressant use (95 for EID-1° and 175 for EID-2°).

### Example 4

In Examples 1-3 above, SID was based on average dosing intervals (ADIs) of >3 to <5 weeks and EID was based on ADIs of >5 to ≤12 weeks. Two pre-specified sensitivity analyses were then performed, which evaluated inclusion of PML cases occurring prior to JCV Ab testing and explored alternative definitions of EID. In the first, PML cases occurring prior to collection of anti-JCV antibody test results in TOUCH were assumed to be anti-JCV antibody positive and added to the three planned analyses described above. In the second sensitivity analysis, alternative EID definitions of ≤13 infusions in the last 18 months and ≤9 infusions over any 12 month period were used for inclusion in the primary and secondary EID analysis groups, respectively. Alternative inclusion criteria for the tertiary analysis were not tested.

The robustness of the three analyses was evaluated to determine the impact of study design decisions on the results. The first sensitivity analysis examined the effect of excluding patients without known anti-JCV antibody status by including PML cases that occurred before 2012 under the assumption that all were anti-JCV antibody positive. This added one EID and 67 SID PML cases to the primary analysis, five EID and 65 SID PML cases to the secondary analysis, and 0 EID and 71 SID PML cases to the tertiary analysis. Using the same post-2012 population denominators as the initial analyses (since anti-JCV antibody status is mostly unknown for the pre-2012 population), HRs for EID vs SID ranged from <0.01 to 0.09 in all three analyses (Table 22).

The second sensitivity analysis investigated the effect of the number of EID doses required for inclusion in EID groups by employing alternative EID eligibility criteria. The risk of PML was significantly lower for EID than for SID using the alternative EID inclusion criteria of ≤13 infusions in the previous 18 months (HR, 0.10; 95% CI, 0.02-0.45) in the primary analysis, or ≤9 infusions over 12 months (HR, 0.01; 95% CI, <0.01-0.09) in the secondary analysis (Table 22). Alternative EID inclusion criteria in the tertiary analysis were not explored.

Two post hoc analyses were carried out to address the impact of potential selection biases on the composition of the EID analysis cohorts. When the effect of excluding patients with dosing gaps (intervals >12 weeks between two infusions) was assessed by including patients with dosing gaps in the three planned analyses of PML risk, the resulting HRs ranged from 0.08 to 0.16 (Table 22).

Although all patients included in this study had tested positive for anti-JCV antibodies at least once, a second post hoc analysis was conducted to evaluate whether the duration of anti-JCV antibody seropositivity affected risk estimates. Longitudinal anti-JCV antibody status (antibody status conversion from negative to positive at some point in time) as a time-varying covariate was incorporated in the Cox regression model. The resulting HR (95% CI) estimates were 0.05 (0.11-0.18) in the primary analysis and 0.11 (0.04-0.26) in the secondary analysis (Table 22). This sensitivity analysis was not performed for the planned tertiary analysis.

EID was associated with a reduction in the conditional risk of PML in each successive epoch of natalizumab treatment for all three definitions of EID and SID (Table 23). Over the first four treatment epochs (<48 infusions), only one PML case (in the secondary analysis) was observed in EID groups; no cases were observed in the primary and tertiary analyses. In the fifth and sixth epochs (49-72 infusions), PML risk was substantially lower for EID than for SID across all three analyses (Table 23).

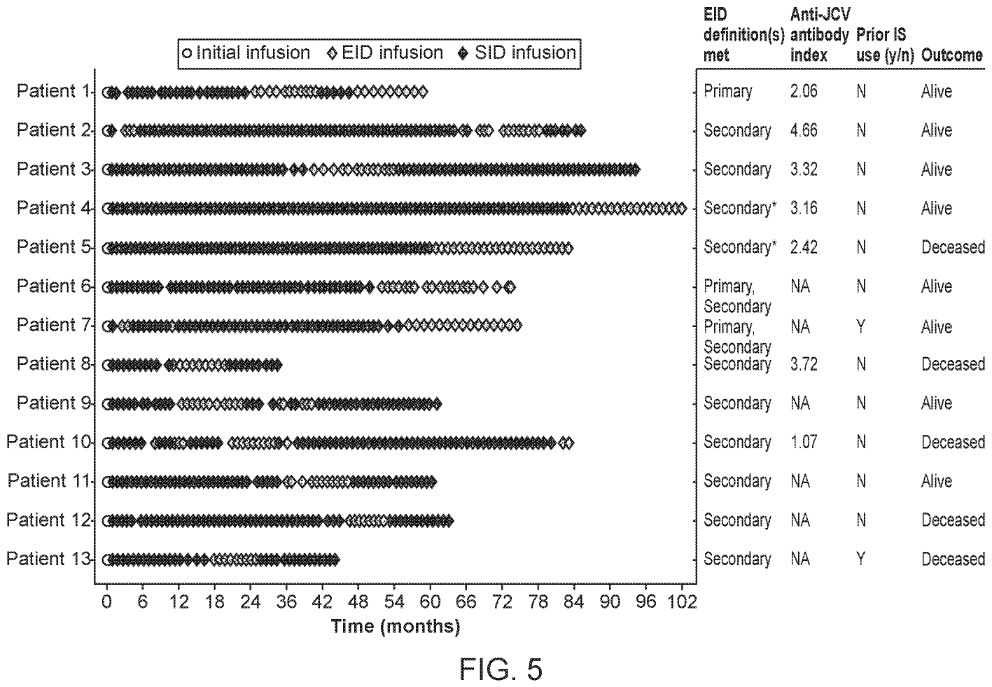

Thirteen PML cases were identified among patients meeting primary and secondary EID inclusion criteria. One case met the primary analysis criteria only, 10 cases met the secondary analysis criteria only, and two cases met criteria for both analyses. There were no PML cases in the tertiary analysis. At the time of PML diagnosis, eight of 13 patients, all of whom were included in the secondary analysis, had switched back to SID from EID and had been on SID for ≥28 weeks immediately before PML diagnosis (FIG. 5). PML patients with a history of EID had longer natalizumab treatment durations, more natalizumab infusions before starting an EID regimen, and more total natalizumab infusions on average than their respective overall EID cohorts (Table 24). Prior immunosuppressant use was also more common in EID PML cases than in the overall EID cohorts (primary analysis: 33% vs 5%; secondary analysis: 17% vs 5%). Of the seven PML cases for whom pre-PML anti-JCV antibody index values were available, six had index values >1.5 (FIG. 5).

Thus, the primary analysis results described in Examples 1-3 are robust to changes in EID interval definition, study inclusion/exclusion criteria, and PML definitions, providing further evidence that, in the US, natalizumab EID is associated with a statistically significant, clinically meaningful PML risk reduction in JCV Ab+ patients compared with natalizumab SID.

TABLE 22
PML hazard ratio (95% CI) for EID vs SID in the sensitivity and post hoc selection bias analyses

| | | | | |
|---|---|---|---|---|
| | | | Post hoc analysis: | |
| | | | inclusion of PML | |
| | | | cases without | |
| | Sensitivity analysis: | Sensitivity | known anti-JCV | Post hoc analysis: |
| | inclusion of PML cases | analysis: | antibody positive | duration of anti- |
| | without known anti-JCV | alternative EID | status and patients | JCV antibody |
| Planned analysis | antibody positive status* | inclusion criteria† | with dosing gaps‡ | positive status§ |

Primary analysis: EID in the last 18 months of treatment
≤15 infusions in the last 18 months

| | | | | |
|---|---|---|---|---|
| EID-1°, n | 1989 | 998 | 7029 | 1988 |
| SID-1°, n | 13 199 | 14 122 | 17 185 | 13 132 |
| PML HR (95% CI) | 0.05 (0.02-0.16) | 0.10 (0.02-0.45) | 0.10 (0.04-0.20) | 0.05 (0.11-0.18) |

Secondary analysis: EID lasting ≥6 months at any time in treatment history
≤10 infusions over 12 months

| | | | | |
|---|---|---|---|---|
| EID-2°, n | 3336 | 1870 | 9593 | 3331 |
| SID-2°, n | 15 489 | 17 902 | 16 282 | 15 424 |
| PML HR (95% CI) | 0.09 (0.04-0.18) | 0.01 (<0.01-0.09) | 0.16 (0.10-0.24) | 0.11 (0.04-0.26) |

Tertiary analysis: majority of treatment received as EID
≤10 infusions/year over the duration of infusion history

| | | | | |
|---|---|---|---|---|
| EID-3°, n | 815 | NA | 6307 | NA |
| SID-3°, n | 23 239 | NA | 27 336 | NA |
| PML HR (95% CI) | <0.01¶ | NA | 0.08 (0.03-0.17) | NA |

CI = confidence interval.
EID = extended interval dosing.
HR = hazard ratio.
JCV = JC virus.
NA = not analysed.
PML = progressive multifocal leukoencephalopathy.
SID = standard interval dosing.
*PML cases assumed to be anti-JCV antibody positive and occurring before 2012 were added to the analysis populations. This added 1 EID and 67 SID cases in the primary analysis, 5 EID and 65 SID cases in the secondary analysis, and 0 EID and 71 SID cases in the tertiary analysis.
†Alternative EID definitions were ≤13 infusions in the last 18 months in the primary analysis and ≤9 infusions over 12 months in the secondary analysis. An alternative definition in the tertiary analysis was not explored.
‡Patients with dosing gaps >12 weeks between infusions were added to the pre-2012 PML case sensitivity analysis cohorts.
§Cox regression modelling of JCV status as a time varying covariate, EID vs SID. This model was not tested in the tertiary analysis.
¶95% CI not estimable because no PML cases occurred in the EID-3° group.

TABLE 23
Life-table estimates of PML risk in patients included in the
primary, secondary, and tertiary analyses

| Natalizumab | | Estimated risk of PML per 1000 patients (no. of cases per adjusted no. of patients) | | | | | |
|---|---|---|---|---|---|---|---|
| exposure* | | Primary analysis | | Secondary analysis | | Tertiary analysis | |
| Expos. | No. of | EID-1° | SID-1° | EID-2° | SID-2° | EID-3° | SID-3° |
| epoch | infusions | group | group | group | group | group | group |
| 1 | 1-12 | 0 (0/1806) | 0 (0/11 890) | 0 (0/2980) | 0 (0/13 049) | 0 (0/662) | 0 (0/18 364) |
| 2 | 13-24 | 0 (0/1659) | 0.28 (3/10 907) | 0 (0/2722) | 0.60 (6/9921) | 0 (0/510) | 0.52 (7/13 425) |
| 3 | 25-36 | 0 (0/1366) | 0.46 (4/8608) | 0.44 (1/2292) | 0.46 (3/6514) | 0 (0/371) | 0.42 (4/9603) |
| 4 | 37-48 | 0 (0/1080) | 2.02 (13/6439) | 0 (0/1841) | 2.58 (12/4650) | 0 (0/265) | 1.79 (13/7254) |
| 5 | 49-60 | 1.23 (1/810) | 3.96 (19/4801) | 1.45 (2/1380) | 4.14 (14/3385) | 0 (0/169) | 3.67 (20/5443) |
| 6 | 61-72 | 1.70 (1/589) | 4.46 (15/3363) | 2.04 (2/980) | 4.74 (11/2323) | 0 (0/104) | 4.16 (16/3848) |

PML risk is shown as the incidence rate per 1000 patients (number of PML cases per adjusted number of patients at risk) in anti-JCV antibody positive patients without prior IS use for the primary and secondary definitions.
Patients with prior IS use could not be analysed due to an insufficient number of patients.
The adjusted number of patients at risk was 95 in the EID-1° group, 689 in the SID-1° group, 171 in the EID-2° group, and 747 in the SID-2° group.
PML risk could not be calculated in the tertiary analysis of EID since no PML cases occurred in this analysis.
Refer to FIG. 4 for definitions of EID and SID under the primary, secondary, and tertiary analyses.
EID = extended interval dosing.
IS = immunosuppressant.
JCV = JC virus.
PML = progressive multifocal leukoencephalopathy.
SID = standard interval dosing.
*Data beyond 6 years are not shown.

TABLE 24
Characteristics of PML patients in EID and SID groups by
primary and secondary PML risk analysis groups

| Natalizumab | | Estimated risk of PML per 1000 patients (no. of cases per adjusted no. of patients) | | | | | |
|---|---|---|---|---|---|---|---|
| exposure* | | Primary analysis | | Secondary analysis | | Tertiary analysis | |
| Expos. | No. of | EID-1° | SID-1° | EID-2° | SID-2° | EID-3° | SID-3° |
| epoch | infusions | group | group | group | group | group | group |
| 1 | 1-12 | 0 (0/1806) | 0 (0/11 890) | 0 (0/2980) | 0 (0/13 049) | 0 (0/662) | 0 (0/18 364) |
| 2 | 13-24 | 0 (0/1659) | 0.28 (3/10 907) | 0 (0/2722) | 0.60 (6/9921) | 0 (0/510) | 0.52 (7/13 425) |
| 3 | 25-36 | 0 (0/1366) | 0.46 (4/8608) | 0.44 (1/2292) | 0.46 (3/6514) | 0 (0/371) | 0.42 (4/9603) |
| 4 | 37-48 | 0 (0/1080) | 2.02 (13/6439) | 0 (0/1841) | 2.58 (12/4650) | 0 (0/265) | 1.79 (13/7254) |
| 5 | 49-60 | 1.23 (1/810) | 3.96 (19/4801) | 1.45 (2/1380) | 4.14 (14/3385) | 0 (0/169) | 3.67 (20/5443) |
| 6 | 61-72 | 1.70 (1/589) | 4.46 (15/3363) | 2.04 (2/980) | 4.74 (11/2323) | 0 (0/104) | 4.16 (16/3848) |

PML risk is shown as the incidence rate per 1000 patients (number of PML cases per adjusted number of patients at risk) in anti-JCV antibody positive patients without prior IS use for the primary and secondary definitions.
Patients with prior IS use could not be analysed due to an insufficient number of patients.
The adjusted number of patients at risk was 95 in the EID-1° group, 689 in the SID-1° group, 171 in the EID-2° group, and 747 in the SID-2° group.
PML risk could not be calculated in the tertiary analysis of EID since no PML cases occurred in this analysis.
Refer to FIG. 4 for definitions of EID and SID under the primary, secondary, and tertiary analyses.
EID = extended interval dosing.
IS = immunosuppressant.
JCV = JC virus.
PML = progressive multifocal leukoencephalopathy.
SID = standard interval dosing.
*Data beyond 6 years are not shown.

### Example 5

The primary objective of this study is to evaluate the efficacy of natalizumab (300 mg IV infusion) extended interval dosing (EID) in subjects who have previously been treated with natalizumab standard interval dosing (SID) for at least 12 months, in relation to continued SID treatment.

The secondary objectives are to evaluate additional relapse-based clinical efficacy measures of natalizumab SID for at least 12 months in relation to continued SID treatment, additional magnetic resonance imaging (MRI)-lesion efficacy measures of natalizumab SID for at least 12 months in relation to continued SID treatment, and the safety of natalizumab SID for at least 12 months in relation to continued SID treatment.

Arms

| Arm Title | Type | Description |
|---|---|---|
| SID | Experimental | Participants will receive |
| | | natalizumab 300 milligram |
| | | (mg) intravenous (IV) |
| | | infusion every 4 weeks |
| | | (−2/+5 days) up to Week 72. |
| EID | Experimental | Participants will receive |
| | | natalizumab 300 mg IV |
| | | infusion every 6 weeks |
| | | (2/+5 days) up to Week 72. |

Outcome Measures

| Outcome Measures | Time Frame | Description |
|---|---|---|
| Number of new or Newly | Week 48 | Number of new or newly |
| Enlarging T2 Hyperintense | | enlarging T2 hyperintense |
| Lesions at Week 48 | | lesions on brain will be |
| | | analysed by magnetic |
| | | resonance imaging (MRI) |
| | | scans of brain. New MRI |
| | | scans will be compared with |
| | | the prior MRI scans to analyse the |
| | | number of new or newly enlarging |
| | | T2 hyperintense lesions. |
| Time to First Relapse as | Up to | A multiple sclerosis (MS) relapse |
| Adjudicated by an | Week 96 | will be defined as the onset of new |
| Independent Neurology | | or recurrent neurological symptoms, |
| Evaluation Committee | | not associated with fever, infection, |
| (INEC) | | severe stress, or drug toxicity, |
| | | lasting at least 24 hours, |
| | | accompanied by new objective |
| | | abnormalities on a neurological |
| | | examination. Only relapses |
| | | confirmed by an INEC will be |
| | | included in the analysis. |
| Number of new Gadolinium | Weeks 24, | Number of new Gd enhancing and |
| (Gd) Enhancing and new T1 | 48 and 72 | new T1 hypointense lesions on |
| Hypointense Lesions at | | brain will be analysed by MRI |
| Weeks 24, 48 and 72 | | scans of brain. New MRI scans will |
| | | be compared with the prior MRI |
| | | scans to analyse number of new Gd |
| | | enhancing and new T1 hypointense |
| | | lesions. |
| Annualized Relapse Rate at | Weeks | An MS relapse will be defined as |
| Weeks 48 and 72 | 48 and 72 | the onset of new or recurrent |
| | | neurological symptoms, not |
| | | associated with fever, infection, |
| | | severe stress, or drug toxicity, |
| | | lasting at least 24 hours, |
| | | accompanied by new objective |
| | | abnormalities on a neurological |
| | | examination. Only relapses |
| | | confirmed by an INEC will be |
| | | included in the analysis. |
| Number of new or Newly | Weeks | Number of new or newly enlarging |
| Enlarging T2 Hyperintense | 24 and 72 | T2 hyperintense lesions on brain |
| Lesions at Weeks 24 and 72 | | will be analysed by MRI scans of |
| | | brain. New MRI scans will be |
| | | compared with the prior MRI scans |
| | | to analyse the number of new or |
| | | newly enlarging T2 hyperintense |
| | | lesions. |
| Percentage of Participants | Up to | An AE is any untoward medical |
| With Adverse Events (AEs) | Week 96 | occurrence in a participant or |
| and Serious Adverse Events | | clinical investigation participant |
| (SAEs) | | administered a pharmaceutical |
| | | product and that does not |
| | | necessarily have a causal |
| | | relationship with this treatment. An |
| | | AE can therefore be any |
| | | unfavorable and unintended sign |
| | | (including an abnormal laboratory |
| | | finding), symptom, or disease |
| | | temporally associated with the use |
| | | of a medicinal (investigational) |
| | | product, whether or not related to |
| | | the medicinal (investigational) |
| | | product. A SAE is any untoward |
| | | medical occurrence that at any dose |
| | | results in death, is a lifethreatening |
| | | event, requires inpatient |
| | | hospitalization or prolongation of |
| | | existing hospitalization, results in a |
| | | significant disability/incapacity or |
| | | congenital anomaly, is a medically |
| | | important event. |

Key Inclusion Criteria:
Ability of the participant to understand the purpose and risks of the study and provide signed and dated informed consent and authorization to use confidential health information in accordance with national and local participant privacy regulations.
Diagnosis of relapsing remitting multiple sclerosis (RRMS) according to the McDonald criteria.
Treatment with natalizumab as disease-modifying monotherapy for RRMS that is consistent with the approved dosing for a minimum of 12 months prior to randomization. The participant must have received at least 11 doses of natalizumab in the 12 months prior to randomization with no missed doses in the 3 months prior to randomization.
Expanded Disability Status Scale (EDSS) <=5.5 at screening.
No relapses in the last 12 months prior to randomization, as determined by the enrolling Investigator.

Key Exclusion Criteria:
Primary and secondary progressive multiple sclerosis (MS).
MRI positive for Gd-enhancing lesions at screening.
Participants for whom MRI is contraindicated (e.g., have a contraindicated pacemaker or other contraindicated implanted metal device, have suffered, or are at risk for, side effects from Gd, or have claustrophobia that cannot be medically managed).
History of any clinically significant (as determined by the Investigator) cardiac, endocrinologic, hematologic, hepatic, immunologic, metabolic (including diabetes), urologic, pulmonary, neurologic (except for RRMS), dermatologic, psychiatric, renal, or other major disease that would preclude participation in a clinical study, in the opinion of the Investigator.
Presence of anti-natalizumab antibodies at screening.

### Example 6

### Introduction

This report provides
a. A detailed retrospective analysis of EID versus SID;
b. A proposal for how to further investigate the efficacy and safety in terms of PML risk reduction of EID relative to SID; and
c. An updated PK/PD modelling taking into account body weight and extended dosing intervals.

### Summary of Data

a. Detailed Retrospective Analysis of EID Versus SID;

The recommended natalizumab dose of the approved Prescribing Information is 300 mg intravenous infusion over 1 hour every 4 weeks. The most important adverse event affecting the natalizumab benefit-risk assessment is the occurrence of progressive multifocal leukoencephalopathy (PML). PML risk is increased with the presence of anti-John Cunningham virus (JCV) antibodies, longer treatment duration, and prior use of an immunosuppressant (IS) therapy. The rigorous assessment of the impact of alternative dosing regimens on PML risk is limited.

In the United States, only prescribers registered in the MS TYSABRI Outreach: United Commitment to Health (TOUCH) Prescribing Program may prescribe natalizumab for the treatment of MS. The TOUCH database captures all natalizumab infusion records, demographic information, prior use of IS therapy, and JCV antibody status (JCV antibody status being available since February 2012), and could provide information on alternative dosing intervals. PML cases are captured in Biogen's TYSABRI Global Safety Database (the PML database). This analysis was designed to evaluate the potential impact of extended interval dosing (EID) compared with standard interval dosing (SID) on PML risk through a retrospective analysis of the TOUCH database and the PML database.

Research objectives

### Primary Objective

Comparison of the risk of PML between patients who received Tysabri on the current label recommended SID and those who received EID during treatment history, based on the TOUCH database.
This objective intended to investigate the effect of EID as a defined treatment regimen on the risk of PML.

### Secondary Objective

Comparison of the risk of PML between patients who have overall exposure to Tysabri consistent with what is expected based on SID and those who have overall exposure consistent with what is expected from EID, based on the TOUCH database.
This objective intended to investigate the effect of reduced overall exposure to Tysabri on the risk of PML.

### Methodology

The TOUCH Prescribing Program data as well as PML cases up to 1 Jun. 2017 were included in this analysis. The data cut-off (DCO) for the PML database was aligned with the exposure data on 1 Jun. 2017. Throughout planning and finalization of the statistical analysis plan (SAP), all Biogen and non-Biogen investigators remained blinded to the PML data. The PML data were merged with the TOUCH Prescribing Program data after the SAP was finalized.

As the TOUCH database reflects real-world clinical practice, patient choices, and daily life considerations, the dosing frequency of natalizumab varies considerably between patients. Three definitions for EID and SID, which differed in the relevant time periods, were assessed to determine the impact of EID on the risk of PML. These 3 EID and SID definitions were based on a dosing frequency of 0.83 doses per month for at least 18 months.

TABLE 25
Definitions of the EID and SID Groups
Definition for Primary Objective

| | |
|---|---|
| 1a | EID: In the last 18 months (548 days, assuming 30.42 days per month¹) of treatment, the total |
| | number of infusions was ≤ 15.0. These patients were included in the EID group. |
| | SID: In the last 18 months (548 days, assuming 30.42 days per month¹) of treatment, the total |
| | number of infusions was ≥ 16.0. These patients were included in the SID group. |
| 1b (for | EID: In the last 18 months (548 days, assuming 30.42 days per month¹) of treatment, the total |
| sensitivity | number of infusions was ≤ 13.0. These patients were included in the EID group. |
| analysis) | SID: In the last 18 months (548 days, assuming 30.42 days per month¹) of treatment, the total |
| | number of infusions was ≥ 14.0. These patients were included in the SID group. |
| | EID: For at least 6 months: the number of infusions in the 365 days prior to each infusion |
| | was ≤ 10.0. |
| 2a | Specifically. if the number of infusions in the 365 days prior to a given infusion was ≤ 10.0 doses |
| | (or the average number of doses per month was ≤ 0.833), then it was considered an EID infusion. |
| | For patients with <1 year of treatment since the initiation of natalizumab, if the average number of |
| | doses per month prior to the infusion was ≤ 0.833, then it was considered an EID infusion. |
| | Patients were included in the EID group if EID infusions continued for ≥ 6 months after the first |
| | EID infusion. |
| | SID: For at least 6 months, the number of infusions in the 365 days prior to each infusion |
| | was ≥ 11.0 (or the average number of doses per month was > 0.833). |
| | If the dosing pattern of a patient contained both ≥ 6 months of ED dosing and ≥ 6 months of SID |
| | dosing, the patient was included in the EID group only. |
| | EID: For at least 6 months, the number of infusions in the 365 days prior to each infusion |
| | was ≤ 9.0. |
| 2b (for | Specifically, if the number of infusions in the 365 days prior to a given infusion was ≤ 9.0 doses |
| sensitivity | (or the average number of doses per month was ≤ 0.75), then it was considered an EID infusion. |
| analysis) | For patients with < 1 year of treatment since the initiation of natalizumab, if the average number |
| | of doses per month prior to the infusion was ≤ 0.75, then it was considered an EID infusion. |
| | Patients were included in the EID group if EID infusions continued for ≥ 6 months after the first |
| | EID infusion. |
| | SID: For at least 6 months, the number of infusions in the 365 days prior to each infusion |
| | was ≥ 10.0 (or the average number of doses per month was > 0.75). |
| | If the dosing pattern of a patient contained both ≥ 6 months of ED dosing and ≥ 6 months of SID |
| | dosing. The patient was included in the EID group only. |

Definition for Secondary Objective

| | |
|---|---|
| 3a | EID exposure group: Patients with an average number of infusions per year of ≤ 10.0 (total |
| | number of infusions/total number of years of treatment of ≤ 10.0) |
| | SID exposure group: Patients with an average number of infusions per year of 10.0 (total number |
| | of infusions/total number of years of treatment of > 10.0). |
| 3b (for | EID exposure group: Patients with an average number of infusions per year of ≤ 9.5 (total number |
| sensitivity | of infusions/total number of years of treatment of ≤ 9.5) |
| analysis) | SID exposure group: Patients with an average number of infusions per year of 11.5 (total number |
| | of infusions/total number of years of treatment of > 11.5). |

For all definitions, the risk of PML in the EID and SID groups was estimated using the life-table method and Kaplan-Meier estimates. Hazards of PML in the EID and SID groups were compared using Cox regression models adjusted for age, gender, prior use of IS therapy, initiation calendar year, and number of infusions.

### Subjects and Sample Size

The analysis population included patients with a known anti-JCV antibody-positive test result at any timepoint.

For the analysis of prior use of IS therapy, IS therapy included the following reported medication terms: azathioprine, azathioprine or mercaptopurine or thioguanine, cyclophosphamide, methotrexate, mitoxantrone, mycophenolate, and Novantrone.

Patients with any incidence of a dosing gap of >12 weeks (defined as an interval >12 weeks between 2 consecutive infusions) or an overdose (defined as an interval <3 weeks between 2 consecutive infusions) were excluded from the analysis.

As of 1 Jun. 2017, the TOUCH database included data from 90,038 patients. After the inclusion and exclusion criteria in Example 4 were applied, the number of patients analyzed for each definition was: definition la, 1988 EID patients and 13,132 SID patients; definition 1b, 998 EID patients and 14,122 SID patients; definition 2a, 3331 EID patients and 15,424 SID patients; definition 2b, 1870 EID patients and 17,902 SID patients; and, definition 3a, 815 EID patients and 23,168 SID patients. Definition 3b was not analyzed because no PML cases were reported for definition 3a and it was therefore not expected that any meaningful results would be observed for the more restrictive definition 3b.

It is estimated that >90 of the total 196 PML cases reported in the US as of 3 Jan. 2017 occurred after the implementation of the anti-JCV antibody status check into the TOUCH information collection form and, thus, occurred in the population with a known anti-JCV status.

With these potential analysis population sizes and the approximate number of PML events, the primary comparison was expected to have approximately 85% power to detect a reduction in the risk of PML, assuming that EID can reduce the risk by ≥50% (i.e., a hazard ratio [HR] of ≤0.5). If the results of the analyses at the planned DCO of 1 Jun. 2017 were inconclusive based on the criteria described in the statistical methods, an updated analysis was to be performed when the number of patients in the EID group of any definition reached twice the size of that at the current DCO.

### Variables and Data Sources

The data sources for this analysis were the TOUCH and the PML database. The analyses included demographic and treatment history data (including age, gender, prior use of IS therapy, total duration of natalizumab treatment, and total number of natalizumab infusions), and PML occurrence.

### Statistical Methods

### Kaplan Meier Time to Event Analyses

For each definition described in Table 25, time to event (PML occurrence) analyses using a KM estimate of cumulative risk were performed for the EID and SID groups, stratified by prior use of IS therapy. Time to event analyses were based on the time since the initiation of natalizumab treatment rather than the number of natalizumab infusions. Within each stratum of prior use of IS therapy (yes/no), a log-rank test was performed to compare the time to event between the EID and SID groups. It should be noted that the analysis was only intended to determine whether the risk of PML in patients who received natalizumab on an EID regimen at some point during their treatment history was reduced compared with patients who received natalizumab in a consistent SID regimen.

Because an EID regimen is generally more likely to be initiated after a period of SID treatment of varied lengths and tends to have a far more limited length of treatment duration, the analyses did not determine the time to event for an EID regimen compared with that for an SID regimen, and did not assess the effect of the extent of SID exposure prior to EID on the risk of PML.

### Cox Regression Time to Event Analyses

For each definition described in Table 22, time to event analyses using a Cox regression model were performed to compare the hazard of PML between the EID and SID groups, with age, gender, cumulative number of infusions, calendar year of the start of natalizumab treatment, and prior use of IS therapy (yes/no) as covariates. Time to event analyses were based on the time since the initiation of natalizumab treatment rather than the number of natalizumab infusions. The HR (EID to SID) estimate and its 95% confidence interval (CI) from a Cox regression model were the primary basis of inference for each definition of EID. Specifically, if the upper limit of the 95% CI of the HR was <1, the EID group was considered to have a lower risk of PML than the SID group. If the point estimate of the HR was ≥0.9 and ≤1.1, the EID and SID groups were considered to have a similar risk of PML. If the lower limit of the 95% CI of the HR was >1, the EID group was to have a higher risk of PML than the SID group.

The validity proportional hazard assumption was checked, and if there was a major deviation from the assumption, then the analysis based on the log-rank test was considered primary.

### Demographics

Baseline demographic characteristics were balanced across the EID and SID groups for each EID definition. The majority of patients (approximately 68%) were female. The median age of patients at first infusion was approximately 43.0 years (range: 6 years to 84 years). Approximately 5% of patients had prior use of IS therapy. All patients had a known anti-JCV antibody-positive test result at some time point during natalizumab treatment. Baseline demographic characteristics were generally consistent with the main analysis population for patients with known PML or who were JCV antibody positive, for patients with known PML or who were JCV antibody positive including those with a treatment gap, and for patients with a treatment gap only.

### Natalizumab Exposure

For each EID definition, the median total duration of natalizumab treatment was longer in the EID groups (43.0 months to 63.0 months [range: 3 to 131 months] across all definitions) than in the SID groups (25.0 months to 45.0 months [range: 1 to 131 months] across all definitions). The median total number of natalizumab infusions was higher in the EID groups (32.0 infusions to 53.0 infusions [range: 2 to 137 infusions] across all definitions) than in the SID groups (26.0 infusions to 46.0 infusions [range: 1 to 142 infusions] across all definitions). The median number of natalizumab infusions prior to the start of the defined EID treatment period for patients in the EID groups was 37.0 infusions (range: 1 to 117 infusions) and 40.0 infusions (range: 1 to 115 infusions) for definitions 1a and 1b, respectively, and 25.0 infusions (range: 1 to 121 infusions) and 30.0 infusions (range: 1 to 122 infusions) for definitions 2a and 2b, respectively. The results from definitions 2a and 2b indicate that the majority of patients had received natalizumab on an SID regimen for >2 years before switching to an EID regimen. The median number of natalizumab infusions prior to the start of the defined SID treatment period for patients in the SID groups was 27.0 infusions (range: 0 to 121 infusions) and 27.0 infusions (range: 0 to 121 infusions) for definitions 1a and 1b, respectively, and 1.0 infusion (range: 1 to 31 infusions) and 1.0 infusion (range: 1 to 21 infusions) for definitions 2a and 2b, respectively. The median number of natalizumab infusions on or after the start of the defined EID or SID treatment period was lower in the EID groups (12.0 infusions to 17.0 infusions [range: 4 to 120 infusions] across definitions 1a, 1b, 2a, and 2b) than in the SID groups (19.0 infusions to 28.0 infusions [range: 6 to 141 infusions] across definitions la, lb, 2a, and 2b). Definition 3a was defined based on an overall mean of exposure; therefore, no EID regimen was defined. As such, the number of natalizumab infusions prior to or after the start of EID treatment could not be calculated. The mean average duration of 2 natalizumab infusions was longer in the EID groups (35.0 days to 43.0 days) than in the SID groups (29.8 days to 30.5 days) for EID definitions la, 2a, and 3a. Natalizumab exposure data for patients with known PML or who were JCV antibody positive, for patients with known PML or who were JCV antibody positive including those with a treatment gap, and patients with a treatment gap only were generally consistent with the main analysis population. However, for definitions 1a and 2a, patients with known PML or who were JCV antibody positive including those with a treatment gap received fewer natalizumab infusions in total (median 41.0 infusions [range: 2 to 132 infusions] and 45.0 infusions [range: 3 to 137 infusions], respectively) than for the main analysis population (median 50.0 infusions [range: 11 to 132 infusions] and 51.0 infusions [range: 6 to 137 infusions], respectively). In addition, for definitions 1a and 2a, patients with known PML or who were JCV antibody positive including those with a treatment gap received fewer natalizumab infusions prior to the start of the defined EID treatment period (median 30.0 infusions [range: 0 to 117 infusions] and 19.0 infusions [range: 1 to 121 infusions], respectively) than for the main analysis population (median 37.0 infusions [range: 1 to 117 infusions] and 25.0 infusions [range: 1 to 121 infusions], respectively). The same trends were observed for definition la and 2a in patients with a treatment gap only. Note: For the sensitivity analyses that included patients with a treatment gap, exposure time was calculated from the first dose to the last dose; the gap period was not removed from the summary of exposure time.

### Results

Each EID group was associated with a clinically and statistically significantly lower risk of PML compared with the SID group in anti-JCV antibody-positive patients.

### Progressive Multifocal Leukoencephalopathy Risk Analyses

The risk of PML for the main analysis population using the life-table method across all EID definitions was lower in the EID groups than in the SID groups, without prior use of IS therapy. The sample size for patients with prior use of IS therapy was insufficient for interpretation. Without wishing to be bound by theory, the present inventors consider that patients classified as having prior use of IS therapy present a similar decrease in PML risk as a result of EID administration.

For each EID definition, sensitivity analyses to evaluate the risk of PML for patients with known PML or who were JCV antibody positive including those with a treatment gap and for patients with a treatment gap only were generally consistent with the main analysis population.

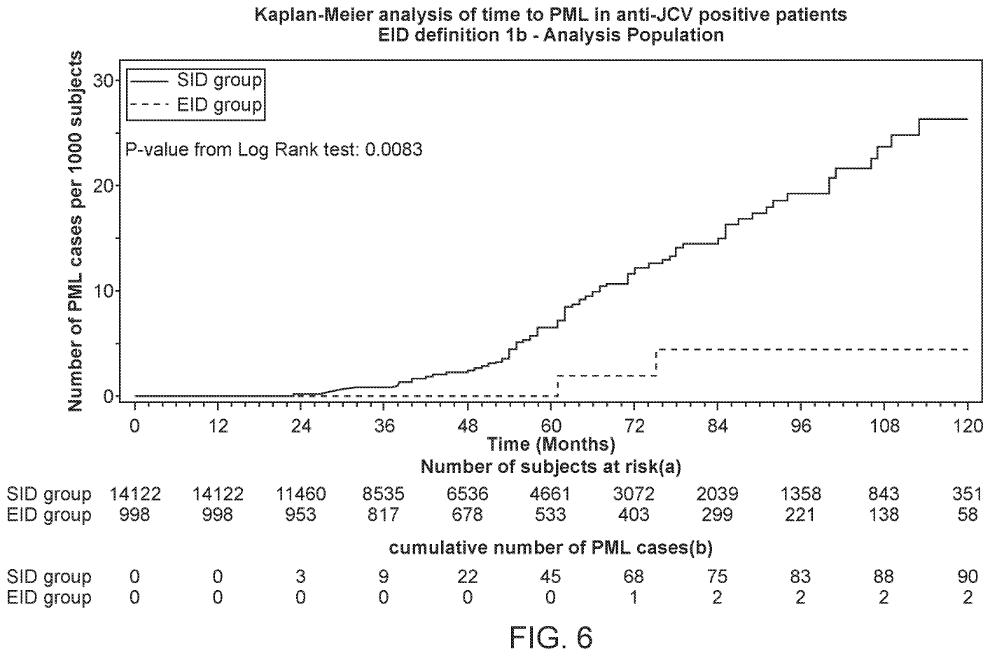

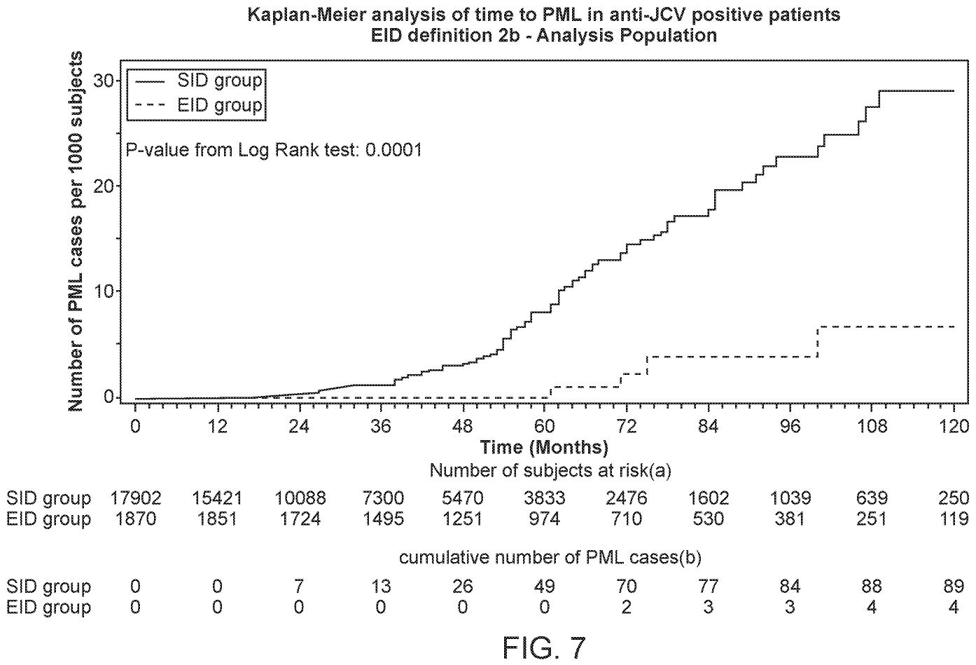

For each EID definition, the KM analysis of the time to PML showed a lower risk of PML for patients in the EID groups than in the SID groups for the main analysis population (FIG. 1D, FIG. 2D, FIG. 3D, and FIGS. 6-7). The KM analysis of time to PML for patients with known PML or who were JCV antibody positive and for patients with known PML or who were JCV antibody positive including those with a treatment was consistent with the main analysis population.

### Hazard of Progressive Multifocal Leukoencephalopathy

For each EID definition, a Cox regression model for time to PML, including the number of cumulative doses as a time-varying covariate, showed that EID treatment was associated with a lower risk of PML than SID treatment, with HR values ranging from 0.014 (95% CI 0.002 to 0.091; p<0.0001) (definition 2b) to 0.122 (95% CI 0.051 to 0.291; p<0.0001) (definition 2a). There were no PML cases reported for definition 3a.

The results for patients with known PML or who were JCV antibody positive, patients with known PML or who were JCV antibody positive including those with a treatment gap, and patients with a treatment gap only were consistent with those for the main analysis population.

Similar trends were observed for the estimated parameters of a Cox regression model for time to PML for each EID definition.

### Discussion and Conclusion

This analysis demonstrates that natalizumab EID treatment is associated with a lower risk of PML than SID treatment in anti-JCV antibody-positive patients. The results are consistent across all EID definitions assessed.

The EID population was primarily composed of patients who had switched to EID treatment after >2 years receiving SID treatment and had a mean average dosing interval of approximately 5 to 6 weeks. Thus, the observed relative decrease in the risk of PML between EID and SID in these analyses is driven by anti-JCV antibody-positive patients after >2 years of natalizumab exposure.

There are a number of limitations and potential biases that should be considered when interpreting the results of these analyses.

The most important limitation is the lack of effectiveness data captured. As such, the benefit-risk of EID treatment compared with SID treatment cannot be assessed in this dataset. Given the effect of EID treatment on the risk of PML observed in this analysis, the marketing authorization holder (MAH) is interested in further investigating the effectiveness of natalizumab EID treatment to better inform on the EID benefit-risk.

In addition, index values are not available in the TOUCH database; therefore, it is unknown how the lower risk associated with EID treatment interacts with the decreased risk of PML associated with lower index values and whether the lack of index information might bias against or toward the observed lower risk of PML with EID treatment compared with SID treatment. As EID treatment is typically utilized in clinical practice in order to lower the risk of PML, it is assumed that index values may be higher in EID-treated patients than in SID-treated patients, which would bias against the treatment group. There are also potential biases in patients who are included for each EID definition. EID-treated patients had on average received more total exposure to natalizumab than SID-treated patients, which would bias against the treatment group. As most patients who switched to EID had been receiving SID for >2 years, the EID-treated patients can be considered as having a selection bias because they were SID-treated patients who did not develop PML while receiving SID prior to switching to EID; this would bias towards the treatment group.

Furthermore, because the risk of PML is increased with the presence of anti-JCV antibodies, only patients with a known anti-JCV antibody-positive test result were included in the analysis population. The TOUCH database only collected JCV antibody status data since late 2012; however, it should be noted that PML patients may have been anti-JCV antibody positive before this time. For the sensitivity analyses, all PML patients were assumed to be anti-JCV antibody positive and were included in the analysis.

Given the robustness, consistency, and magnitude of the difference between EID- and SID-treated patients in terms of PML risk, it is considered highly unlikely that the potential biases described in this section would impact the conclusion that the risk of PML is lower with EID treatment than with SID treatment in patients at, e.g., high, risk of PML, such as anti-JCV antibody positive patients.

As the TOUCH Prescribing Program does not collect effectiveness data, additional studies may establish whether the effectiveness of natalizumab is maintained with EID treatment and to better inform on the EID benefit-risk.

The results of this analysis are not generalizable because it is unclear whether the observed effect of EID treatment on the risk of PML is maintained in different patient populations (e.g., patients with different body weights and other patient populations where the risk of PML may be higher than that in the US population).

b. Further Methods to Increase Efficacy and Safety in Terms of PML Risk Reduction of EID Relative to SID;

In view of the foregoing results from the TOUCH analysis comparing patients treated with standard interval dosing (SID) and patients treated with extended interval dosing (EID) for ≥6 months, the present inventors have developed a multi-phased approach to increase the efficacy and safety of EID relative to SID. The approach is considered suitable for clinical validation in a Randomized, Controlled, Open-Label, Rater-Blinded clinical study. This includes retrospective analyses of established databases through sponsor research agreements and/or MAH led retrospective collaboration, and a prospective, randomized, controlled study.

### A Randomized, Controlled, Open-Label, Rater-Blinded Clinical Study

The primary objective of the study is to evaluate the efficacy of EID of natalizumab after at least 12 months on SID in relation to continued SID with a primary goal of estimating ΔSID-EID with high precision, narrow 95% CI, and adequate power to detect small but clinically relevant differences.

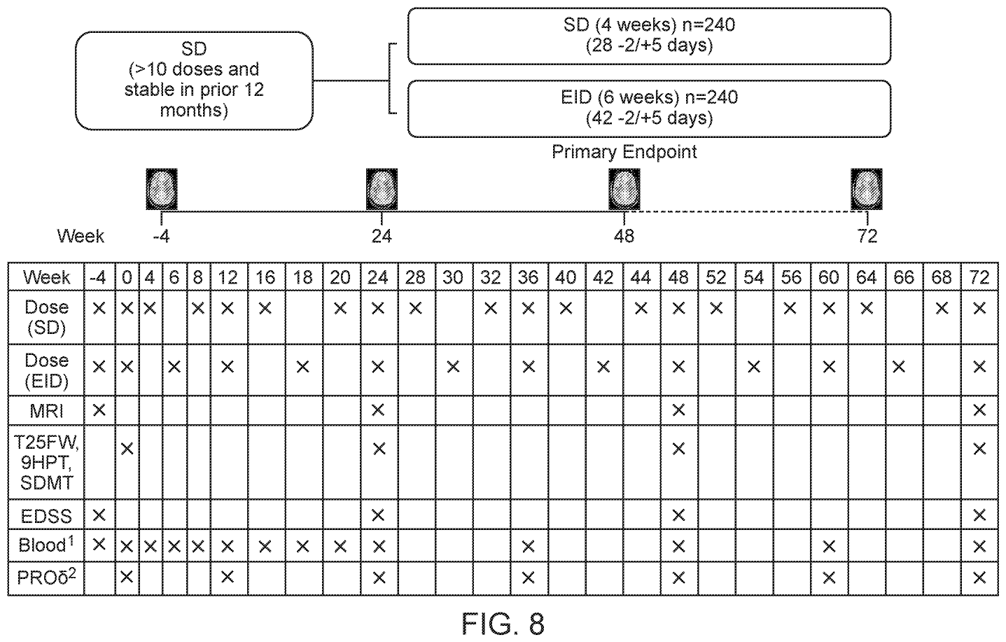

A schematic of this prospective study design is presented as FIG. 8 with the following Study Endpoints:

### Primary Endpoint:

Number of N/NE T2 lesions at week 48
Blinded Central reading provides objective outcome measure, versus relapse, when blinding is not feasible
N/NE T2 is a cumulative measure versus Gd+ lesions—a more transient measure

### Secondary Endpoints:

Clinical outcomes: Time to first relapse, ARR, at week 48 and week 72 and safety
MRI: Number of new or newly enlarging T2 lesions at week 24 and 72, Number of new Gd+enhancing and new T1 hypointense lesions at week 24, 48, 72

PK, PD, and Exploratory Biomarker endpoints:
α4-integrin saturation
natalizumab concentration
lymphocyte subsets

### Study Population:

### Key Inclusion Criteria

Age 18-60, RMS, EDSS ≤5.5
Stable on SID natalizumab for at least 12 months
JCV− and JCV+ included
Disease activity pre-natalizumab: per local label
No relapses in the last 12 months prior to randomization

### Key Exclusion Criteria

Prior immunosuppressant use
Gd+ lesions at screening MRI
Primary and secondary progressive MS

This is a prospective, randomized, interventional, controlled, open-label, rater-blinded, Phase 3b study in subjects with RRMS who have been receiving natalizumab SID for at least 12 months without relapses in the last 12 months. Subjects are randomized to 1 of 2 arms to continue to receive natalizumab at either (1) 4-week intervals, or (2) 6-week intervals.

Randomization is stratified by country/region, body weight (>90 kg versus ≤90 kg) and duration of natalizumab exposure (>3 years versus ≤3 years). All MRI scans are read at a central facility with raters blinded to subject assignment.

Subjects are screened at a regularly scheduled natalizumab dose visit (±3 days) and enrolled and randomized at their next monthly visit if they do not have disease activity as evidenced by Gd+ enhancing lesions on MRI at Screening and meet all other eligibility criteria. Subjects receive open-label natalizumab at their assigned frequency (±3 days) throughout the 72 weeks of the study.

### Study Rationale:

The safety and efficacy of the currently recommended dose (300 mg natalizumab administered intravenously (IV) every 4 weeks) has been well established through clinical trials and in real-world clinical practice.

Extending the dosing interval of natalizumab (e.g., to 6 weeks) has been practiced by some physicians in anti-JCV antibody-positive patients. In a prespecified, retrospective analysis of anti-JCV antibody-positive patients treated with natalizumab in the US TOUCH (Tysabri® Outreach: United Commitment to Health) program (n=18,755), the risk of PML was compared between patients treated with standard interval dosing (SID) and patients treated with extended interval dosing (EID) for at least 6 months (300 mg with an average dosing interval of 5 to 6 weeks). The majority of patients were treated with SID for at least 1 year before switching to EID (median of 25 SID infusions prior to switch). The analysis demonstrated a clinically and statistically significant reduction in the risk of PML in patients treated with EID. In some embodiments, whether extending the dosing interval of natalizumab can improve benefit/risk for patients is established in the prospective, randomized, controlled study described herein.

This study assesses the efficacy, tolerability, and safety of switching to EID (e.g., dosing interval of 6 weeks) after at least 12 months of disease stability on SID (dosing interval of 4 weeks).

This prospective study is designed to generate high quality efficacy data with the goal of estimating the difference between SID and EID, with high precision with a narrow 95% confidence interval, and adequate power to detect small but clinically relevant differences. While this data generation effort will take more time, it will ultimately provide higher level evidence than is possible from PK/PD modelling and registry analyses.

The study will be run in the USA, Canada; Germany; Italy; UK; Spain; France; Netherlands; Belgium, Australia and New Zealand. The first patient is planned to be enrolled in Q1 2019 and the final results are expected in Q2 2021.

### Study Objectives and Endpoints:

### Primary Objective and Endpoint:

The primary objective of the study is to evaluate the efficacy of natalizumab EID in patients who have previously been treated with natalizumab SID for at least 12 months, in relation to continued SID treatment, with the goal of estimating the difference between SID and EID with high precision with narrow 95% CI, and adequate power to detect small but clinically relevant differences.

The primary endpoint that relates to this objective is the number of new or newly enlarging T2 hyperintense lesions at 48 weeks.

### Secondary Objectives and Endpoints are as Follows:

To evaluate additional relapse-based clinical efficacy measures of natalizumab EID in patients who have previously been treated with natalizumab SID for at least 12 months, in relation to continued SID treatment.

To evaluate additional MRI-lesion efficacy measures of natalizumab EID in patients who have previously been treated with natalizumab SID for at least 12 months, in relation to continued SID treatment.

To evaluate safety of natalizumab EID in patients who have previously been treated with natalizumab SID for at least 12 months in relation to continued SID treatment.

The Secondary Endpoints that Relate to These Objectives are:
Time to first relapse (relapses will be adjudicated by an Independent Neurology Evaluation Committee)
Annualized Relapse Rate at Week 48 and Week 72
Proportion of patients relapsing at Week 48 and Week 72
Number of new or newly enlarging T2 lesions at Week 24 and Week 72
Number of Gd+ enhancing and T1 hypointense lesions at Weeks 24, 48, and 72
Safety assessments of adverse events (AEs) and serious adverse events (SAEs)

### The Exploratory Objectives and Endpoints are as Follows:

To evaluate additional clinical and MRI efficacy, safety, and tolerability measures that may be important for clinicians when making treatment decisions for natalizumab EID in patients who have previously been treated with natalizumab SID for at least 12 months, in relation to continued SID treatment.

To evaluate pharmacokinetics (PK), pharmacodynamics (PD), biomarkers, and patient-reported outcomes (PROs) to allow for analyses on optimal responders to EID switching in patients previously treated with natalizumab SID for at least 12 months.

The Exploratory Endpoints that Relate to these Objectives are:

### Exploratory Clinical Efficacy Outcomes:

Expanded Disability Status Scale (EDSS) progression (sustained for 3 months) at Week 48 and Week 72
EDSS improvement (sustained for 3 months) at Week 48 and Week 72
9-hole peg test (9HPT) time, timed 25-foot walk (T25FW) time, and Symbol Digit Modalities Test (SDMT) score at Week 48 and Week 72

### Exploratory Safety and Tolerability Outcomes:

Anti-natalizumab antibodies at Screening and Weeks 12, 24, 36, 48, 60, and 72
Anti-JCV antibody status assessed at Baseline and Weeks 24, 48, and 72

### Exploratory MRI Outcomes:

Volume of Gd+ enhancing, T2 hyperintense, and non-enhancing T1 hypointense lesions at Week 48 and Week 72
Percentage of brain volume change (PBVC) at Week 48 and Week 72 Exploratory PK, PD,

### Biomarker, and PRO Outcomes:

Serum trough natalizumab concentrations (Ctrough)
Trough α4-integrin saturation
Lymphocyte counts with lymphocyte subsets, including T cells, B cells, and NK cells (CD4, CD8, CD19, and CD56)
Additional serum, plasma, whole blood RNA, and PBMC samples will be collected and stored for future potential testing of exploratory markers related to natalizumab treatment response or MS disease biomarkers, to be tested at the Sponsor's discretion.
For example, exploratory biomarkers may include, but are not limited to, serum neurofilament light chain, soluble vascular cell adhesion molecule 1 (VCAM 1), α4-integrin expression, etc.
PROs: Treatment Satisfaction Questionnaire for Medication (TSQM), Neurology Quality of Life (Neuro-QoL) fatigue questionnaire, Multiple Sclerosis Impact Scale (MSIS-29), EuroQol 5 dimensions questionnaire (EQ-5D-5L), and clinical global impression scale (physicians and patients) at Weeks 12, 24, 36, 48, 60, and 72.

### Exploratory CSF Substudy Objectives and Endpoints are as Follows:

To explore if EID leads to evidence of CNS lymphocyte activity in the absence of MS disease activity.

For a selected number of sites with lumber puncture capability, CSF will be collected from consenting subjects and stored for future potential testing of exploratory markers related to natalizumab treatment response or MS disease biomarkers, to be tested at the Sponsor's discretion. For example, exploratory biomarkers may include, but are not limited to, natalizumab concentration, lymphocyte counts, lymphocyte subsets, neurofilament light levels, immunoglobulin index oligoclonal bands (OCBs), or other markers of inflammation, etc.

### Rationale for Dose and Schedule Selection:

The recommended dose of natalizumab is 300 mg intravenous (IV) infusion every 4 weeks. In a prespecified retrospective analysis of anti-JCV antibody-positive patients treated with natalizumab in the United States (n =18755), the risk of PML was compared between patients treated with SID and patients treated with EID (300 mg with an average dosing interval of 5 to 6 weeks). The majority of patients were treated with SID for at least 1 year before switching to EID. The analysis demonstrated a clinically and statistically significant reduction in the risk of PML in patients treated with EID. This study assesses the efficacy of switching to EID (e.g., dosing interval of 6 weeks) in relation to remaining on the recommended dose.

### Duration of Study Participation:

An individual subject participates in this study for 88 weeks including a 4-week screening period, 72 weeks of randomized treatment, and a follow-up period of 12 weeks.

Follow-up Period: 12 weeks

### Study Location:

Approximately 80 sites in North America (approximately 70%), Europe (approximately 25%), and Australia (approximately 5%) are planned.

### Number of Planned Subjects:

Approximately 480 subjects are expected to be enrolled. The subjects are randomized in a 1:1 ratio to SID or EID treatment. The randomization is stratified by country/region, body weight (<90 kg versus >90 kg), and duration of natalizumab exposure (>3 years versus <3 years).

### Sample Size Determination:

To detect an increase in the mean number of new or newly enlarging T2 lesions over 48 weeks at the alpha level of 0.05 (2-sided), a sample size of 400 subjects (200 subjects per arm) provides the following:
>80% power to detect an increase from a mean of 0.3 (expected efficacy of SID dosing arm in this population) to 0.5
≥90% power to detect an increase from a mean of 0.3 to 0.6.

Historical data on MS treatments, including meta-analyses on the relationship between new or newly-enlarging T2 lesions and relapses suggest little or no clinical relevance of a difference smaller than 0.2 in mean new or newly-enlarging T2 lesions over 48 weeks. Approximately 480 subjects will be enrolled to account for a drop-out rate of approximately 17%.

### Study Population:

### Inclusion Criteria

To be eligible to participate in this study, candidates must meet the following eligibility criteria at the time of randomization, or at the time point specified in the individual eligibility criterion listed:
1. Ability of the subject to understand the purpose and risks of the study and provide signed and dated informed consent and authorization to use confidential health information in accordance with national and local subject privacy regulations.
2. Aged 18 to 55 years old, inclusive, at the time of informed consent.
3. Diagnosis of RRMS
   Treatment with natalizumab that is consistent with the approved dosing for a minimum of 12 months prior to randomization. The subject must have received at least 11 doses of natalizumab in the 12 months prior to randomization with no missed doses in the 3 months prior to randomization.
4. EDSS <5.5 at Screening.
5. No relapses in the last 12 months prior to randomization, as determined by the enrolling Investigator.
6. All women of childbearing potential must practice effective contraception during the study and for 3 months after their last dose of study treatment.

### Exclusion Criteria

Candidates will be excluded from study entry if any of the following exclusion criteria exist at the time of randomization, or at the time point specified in the individual criterion listed:
1. Known history of human immunodeficiency virus.
2. Known history of hepatitis C (test for hepatitis C virus antibody [HCV Ab]) or hepatitis B virus (test for hepatitis B surface antigen [HBsAg] and/or hepatitis B core antibody [HBcAb]).
3. MRI positive for Gd-enhancing lesions at Screening.
4. Subjects for whom MRI is contraindicated (e.g., have a contraindicated pacemaker or other contraindicated implanted metal device, have suffered, or are at risk for, side effects from Gd, or have claustrophobia that cannot be medically managed).
5. History of any clinically significant (as determined by the Investigator) cardiac, endocrinologic, hematologic, hepatic, immunologic, metabolic (including diabetes), urologic, pulmonary, neurologic (except for RRMS), dermatologic, psychiatric, renal, or other major disease that would preclude participation in a clinical study, in the opinion of the Investigator.
6. History of malignant disease, including solid tumors and hematologic malignancies (with the exception of basal cell and squamous cell carcinomas of the skin that have been completely excised and are considered cured).
7. History of transplantation or any anti-rejection therapy.
8. History of severe allergic or anaphylactic reactions or known hypersensitivity to any antibody drug therapy.
9. A clinically significant infectious illness (e.g., cellulitis, abscess, pneumonia, septicemia) within 30 days prior to Screening, or PML or other opportunistic infections at any time.
10. Presence of anti-natalizumab antibodies at Screening.
11. Signs or symptoms suggestive of any serious infection, based on medical history, physical examination, or laboratory testing, as determined by the Investigator.

### Treatment History

12. Prior treatment with cladribine, mitoxantrone, T-cell or T-cell receptor vaccination, cyclophosphamide, cyclosporine, azathioprine, methotrexate, or mycophenolate mofetil.
13. Prior treatment with any therapeutic monoclonal antibody other than natalizumab within 24 months prior to randomization.
14. Prior treatment with total lymphoid irradiation.
15. Prior treatment with IV immunoglobulin (IVIg), plasmapheresis, or cytapheresis within 12 months prior to randomization.
16. Treatment with IV or oral corticosteroids (topical corticosteroids are acceptable) or related products within 3 months prior to randomization.

### Miscellaneous

17. Female subjects considering becoming pregnant while in the study or who are pregnant or currently breastfeeding.
18. History of drug or alcohol abuse within 2 years prior to entry, per Investigator judgment.
19. Current enrollment in any other study treatment or disease study.
20. Inability to comply with study requirements.
21. Other unspecified reasons that, in the opinion of the Investigator or Biogen, make the subject unsuitable for enrollment.

### Rescreening Criteria:

Subjects who fail screening due to transient infection can be rescreened once, within 30 days.

### Treatment Groups:

SID group: approximately 240 subjects receive natalizumab as a 300 mg IV infusion every 4 weeks (28±3 days).

EID group: approximately 240 subjects receive natalizumab as a 300 mg IV infusion every 6 weeks (42±3 days).

Visit Schedule: A given subject can expect no more than a total of 21 visits to the clinic during this study including Screening and Follow-up.

### Definition of Dose-Limiting Toxicity:

The definition of dose-limiting toxicity is not applicable.

### Individual Subject Rescue and Therapy Options

Rescue treatment is indicated for a subject if any one or more of the following conditions are determined:
4 or more new or enlarging T2 lesions of any size, compared with a previous scan performed as part of this study
An increase of ≥1.5 in EDSS as compared with previous assessment (confirmed at least 12 weeks after the initial increase)
2 clinical relapses with new or recurrent neurological symptoms, not associated with fever or infection, having a minimum duration of 24 hours and either of the following: 1) an increase of ≥1 grade in ≥2 functional scales of the EDSS; or 2) an increase of ≥2 grades in 1 functional scale of the EDSS; or 3) an increase of ≥1 in the EDSS if the previous EDSS was ≤5.5, or ≥0.5 if the previous EDSS was ≥6 (an increase of ≥1.5 in EDSS if previous EDSS is equal to 0).

If a subject treated with natalizumab EID experiences any of the above conditions, the Investigator can revert to natalizumab SID as a rescue treatment within 4 weeks of either the date of MRI that showed the disease activity or confirmation of EDSS progression for at least 3 months, at his/her discretion. Subjects who receive rescue therapy different from natalizumab SID are withdrawn from the study.

For subjects who experience acute clinical relapse, high-dose corticosteroid treatment as per local standard of care (up to 5 days) for relapse treatment may be administered.

### Discontinuation of Treatment:

A subject must permanently discontinue study treatment for any of the following reasons:
The subject becomes pregnant. Study treatment must be discontinued immediately and pregnancy should be reported.
The subject develops persistent anti-natalizumab antibodies (2 consecutive readings).
The subject develops PML.
The subject withdraws consent.
The subject experiences a medical emergency that necessitates permanent discontinuation of study treatment.
At the discretion of the Investigator for medical reasons
At the discretion of the Investigator or Sponsor for noncompliance.

Subjects who discontinue study treatment may remain in the study and continue protocol-required tests and assessments. If a subject chooses to withdraw from the study, an Early Termination Visit should occur as soon as possible but no later than 4 weeks after the last dose of study treatment; in addition, all End of Study (EOS) assessments should be conducted at a separate EOS visit 12 weeks (±3 days) after the final dose of study treatment is received. The primary reason for discontinuation of study treatment must be recorded in the subject's electronic case report form.

### Efficacy Assessments:

### MRI Efficacy Assessments

T2 hyperintense lesion number and volume
Gd+ enhancing lesion number and volume
T1 hypointense lesion number and volume
PBVC

### Clinical Efficacy Assessments

Relapses (clinical relapses are assessed as defined by new or recurrent neurologic symptoms not associated with fever or infection having a minimum duration of 24 hours)
Neurological examination and EDSS
9HPT, T25FW, SDMT
TSQM, Neuro-QoL fatigue, MSIS-29, EQ-5D-5L
   Safety Assessments: AEs, SAES, anti-natalizumab antibodies, and anti-JCV antibody status.

### Study Treatment Concentration Assessments:

Serum trough natalizumab concentration.

### Pharmacodynamic Parameters:

Trough α4-integrin saturation.
Lymphocyte counts with lymphocyte subsets, including T cells, B cells, and NK cells (CD4, CD8, CD19, CD34, and CD56).
Serum, plasma, whole blood RNA, PBMC, CSF (optional), and DNA (optional) will be collected and stored for future potential testing of exploratory markers related to natalizumab treatment response or MS disease biomarkers, to be tested at the Sponsor's discretion.

### DNA/RNA/Proteomic Sample Collection:

Rationale: It may be important to develop potential predictive and/or pharmacodynamic markers of treatment-related responses to the EID regimen, to support selection of the appropriate dose regimen that optimizes benefit/risk for the patient. Samples are collected and stored for optional whole blood RNA expression and/or proteomic profiling of MS disease activity and/or natalizumab treatment response (testing to be performed at the Sponsor's discretion). DNA samples are collected from consenting subjects to optionally identify genes that may influence response to natalizumab or MS disease course. Participation in genetic studies is optional for patients, and the pharmacogenomic analysis is exploratory (testing to be performed at the Sponsor's discretion). Potentially, some heterogeneity in clinical response to treatment may be associated with genetic variation in patients. However, there are currently no data suggesting that specific genetic polymorphisms are associated with response to natalizumab.

### Samples for Laboratory Assessments:

No duplicate samples are taken; however, aliquots of the original samples are stored as back-up in case the original sample is lost or not evaluable.

### Statistical Statement and Analytical Plan:

The primary endpoint, new or newly-enlarging T2 lesions at Week 48, is analyzed using negative binomial regression models with treatment as the classification variable and body weight (≤90 kg versus >90 kg), EDSS, and region as covariates.

The ratio of mean lesion numbers of SID versus EID (EID/SID) is derived from the model with a 95% confidence interval (CI) and associated p-value. The treatment can be considered different if the p-value (2-sided) is less than 0.05. The possibility of a 2-fold increase in the mean lesion number in the EID group as compared to the SID group can be considered ruled out if the upper limit of 95% CI is less than 2. The proportion of patients with no new or newly-enlarging T2 lesions is analyzed using logistic regression models with the same covariates as described above. New or newly-enlarging T2 lesions at other time points as well as Gd+ lesions are analyzed similarly. Key secondary endpoints of relapse are also analyzed using negative binomial regression models. Time to event endpoints is analyzed using the Cox regression model as well as Kaplan-Meier estimates. Performance test outcomes and PD biomarkers are analyzed using the Mixed Model of Repeated Measures. The relationship between PK concentration and efficacy endpoints (MRI lesions and relapse) as well as between PD biomarkers and efficacy endpoints are assessed using negative binomial regression or logistic regression models. Treatment differences in subgroups defined by PK concentration categories and a-integrin saturation level categories are also be assessed.

The incidence of AEs during the randomized treatment period is tabulated by treatment group, severity, and relationship to study treatment. The tabular summaries include incidence by system organ class and by preferred term. AEs and SAEs resulting in study withdrawal are summarized by treatment group. Clinically relevant abnormalities for laboratory parameters are identified by treatment group.

### Interim Analysis:

An interim futility analysis, based on the number of new or newly enlarging T2 lesions at 6 months, is conducted when 50% of subjects have >6 months of randomized treatment. The interim futility analysis is designed to ensure that if clinically meaningful loss of efficacy is occurring in the EID group, the study can be stopped early to prevent unnecessary risk to the study participants of uncontrolled MS disease activity as a result of potential efficacy failure on EID.

### Study Stopping Rules:

The study is stopped if the interim futility analysis shows a statistically significant worsening of efficacy as gauged by new or newly-enlarging T2 lesions in the EID group relative to the SID group. This study may be terminated, after informing Investigators, at any time. Investigators will be notified if the study is placed on hold, completed, or closed.
End of Study: The end of study is last subject, last visit.
Go/No Go Criteria: Not applicable
   c. An Updated PK/PD Modelling taking into Account Body Weight and Extended Dosing Intervals;

Data and modelling and simulation results are provided below.

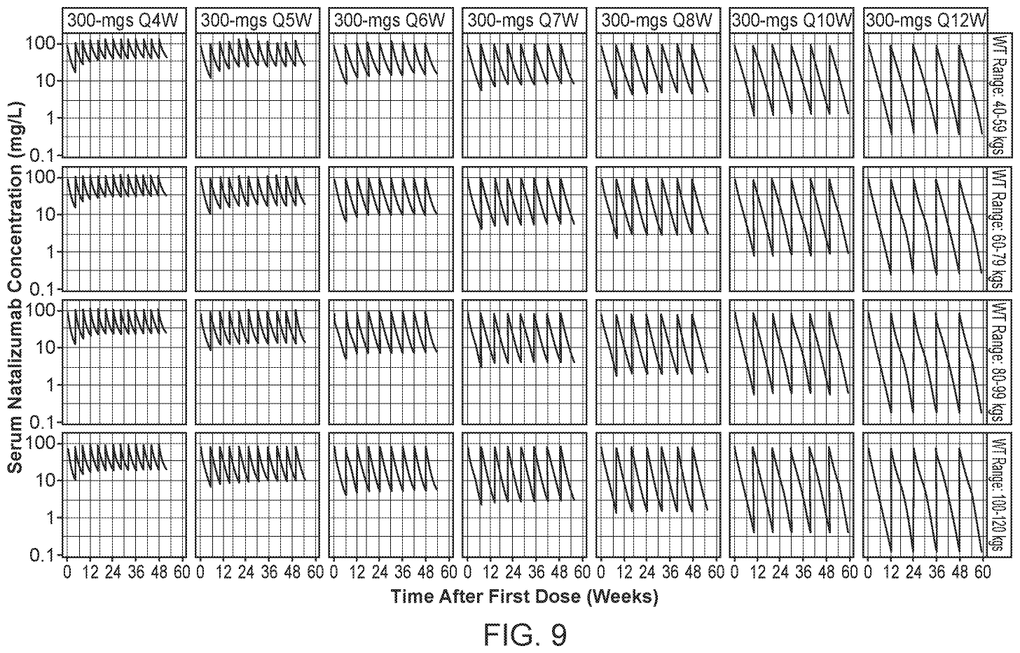

Simulation results for Cavg, Ctrough, and integrin saturation (%) at 1 year of natalizumab treatment for each of the following dose regimens: 300 mg every 4 weeks (Q4W), every 5 weeks (Q5W), every 6 weeks (Q6W), every 7 weeks (Q7W), every 8 weeks (Q8W), every 10 weeks (Q10W), and every 12 weeks (Q12W), are shown in FIG. 9.

Predictions are presented for each of 4 body weight groups (40 to <60 kg, 60 to <80 kg, 80 to <100 kg, and 100 to <120 kg). Time courses of mean serum natalizumab concentrations and mean integrin saturation (%) over 1 year of treatment (0 to 52 weeks) are plotted for all treatment and body weight groups.

As indicated, previously published PK-PD models with clinical endpoints (Gd+ lesion counts, ARR, etc.) (Muralidharan 2017) were developed for Tysabri-naïve individuals and designed to evaluate Q4W dosing with various dose levels. As such, these models cannot directly be applied to simulate a situation in which individuals have been on the label dosing regimen of 300 mg Q4W for some time and then transferred to a dosing with a different frequency (e.g., Q6W or Q8W). Without wishing to be bound by theory, the present inventors consider that there are apparent PD delays in the system, e.g., decline of integrin saturation, and the recurrence of Gd+ lesions after the cessation of standard therapy did not occur for some time in spite of a rapid decline in PK concentration. Such apparent delays are unaccounted for in PK-efficacy models as they would not have mattered when Tysabri-naive individuals were simulated. However, in modelling the efficacy of natalizumab in patients who are stable on Q4W dosing and transition to Q6W dosing, these apparent delays need to be taken into consideration.

In this study, the results of an updated modelling approach that addresses this apparent delay and the subsequent simulations from the models are provided. In summary, the updated simulation results show that efficacy, in terms of integrin saturation, is maintained at high levels across all body weights with Q4W and Q5W dosing. Efficacy starts to decrease more appreciably in the 2 higher weight categories with Q6W and is progressively worsened with less frequent dosing.

### METHODS

### Data for Modelling

Study 101MS205 (RESTORE) was a prospective randomized study in patients with relapsing forms of multiple sclerosis (MS) who have been receiving natalizumab treatment for at least 12 months with no MS relapses for at least 12 months prior to randomization. Patients who had received 300 mg natalizumab IV for >12 months prior to trial entry were randomly assigned at a 1:1:2 ratio to natalizumab, placebo, or alternate therapy (intramuscular interferon beta-1a, glatiramer acetate, or methylprednisolone), respectively. Randomization occurred at Week 0 (at which time all patients received their last natalizumab infusion). At Week 28, patients discontinued placebo or alternate therapy and restarted open-label natalizumab. In an analysis conducted to characterize the timing of the PK and PD changes after the cessation of natalizumab treatment in this study, mean trough natalizumab concentrations in patients whose natalizumab treatment was interrupted declined rapidly from 38.4 μg/mL at Week 4 after cessation to 3.8 μg/mL at Week 12 [Plavina 2017]. The α4-integrin saturation level, however, declined at a slower rate, from a mean of 89.4% at Week 4 to 31.3% at Week 12, and then plateaued at approximately 10% to 15% from Week 16 onward. In patients whose natalizumab treatment was interrupted, none had Gd+ lesions that met the magnetic resonance imaging (MRI) recuse criteria (1 lesion of >0.8 cm3 in volume or ≥2 lesions of any size) at Week 4 or Week 8.

However, the proportion of patients that have Gd+ lesions that met the MRI recuse criteria increased to 2.5% at Week 12 and further increased to 41.8% at Week 16. Clinical relapse in patients whose natalizumab treatment was interrupted also occurred at an increased frequency after Week 12 [Fox 2014]. The data of RESTORE suggest that, in patients who are stable on natalizumab treatment for at least 12 months, the decline in α4-integrin saturation level upon cessation of natalizumab appears to be delayed compared with the drug concentration level, and the return of disease activity appears to correspond to the rate of decline in saturation level.

To assess the relationship, the data of a4-integrin saturation level; Gd+ lesions at Weeks 4, 8, 12, and 16; and the occurrence of clinical relapse up to Week 16 from this population of patients who were previously stable on natalizumab treatment for ≥12 months in RESTORE study were used for modeling. As a large proportion of patients with natalizumab interruption restarted natalizumab after Week 16 following the return of disease activity [Fox 2014], the data after Week 16 were excluded from modelling.

It should be noted that Study 101MS206 (REFINE) was a prospective randomized study to evaluate various dosing regimens of natalizumab additionally in patients who have been receiving natalizumab at the standard doing regimen for at least 12 months with no MS relapse. The studied regimens included 300 mg IV Q4W and Q12W regimens. However, as the MRI scans, neurological examinations, and blood collections for α4-integrin saturation level were performed at 12-week intervals, REFINE data were not used as part of the model building data set, but rather as a validation data set of the simulation results for Q4W and Q12W dosing.

### Model 1: Probability of Gd+ Lesion Occurrence

A generalized estimating equation (GEE) model that includes the lesion occurrence (Yes/No) at Weeks 4, 8, 12, and 16 as a repeated measure correlated response variable and α4-integrin saturation level at corresponding timepoints as the explanatory variable was used to fit the data.

The mean response was modeled using logit link function with an exchangeable working correlation matrix between timepoints. Specifically, suppose γij represents the Gd+ lesion status of patient i at the jth visit, j=1, . . . , 4, the model for the probability of lesion occurrence μij is g(μij)=β0+xijβ1 where xij is the saturation level of patient i at the jth visit and the logit link function g(μij)=log(μij/[1−μij]) with a binomial underlying distribution. Predicted values of μ (probability of lesion occurrence) for given saturation levels were derived from the model with robust estimate of variance.

For patients who have met the MRI rescue criteria, their response values at subsequent visits were imputed as “Y” regardless of the actual scan results.

### Model 2: Mean Number of Gd+ Lesions

A similar GEE model was also built to assess the relationship of the lesion counts at Weeks 4, 8, 12, and 16 with the a4-integrin saturation level at corresponding time points. A log link function with a negative binomial underlying distribution was used in the model for the mean number of lesions. An exchangeable working correlation matrix between time points was also assumed.

Predicted values of μ (mean number of lesions) given saturation levels were derived from the model with robust estimate of variance.

For patients who have met the MRI rescue criteria, their response values at subsequent visits were imputed using the number of lesions observed at the time of rescue regardless of actual scan results.

### Model 3: Probability of Occurrence of Clinical Relapse

The probability of the occurrence of clinical relapse was also assessed using a similar GEE model with the occurrence (Yes/No) of relapse over the period of Weeks 0 to 4, Weeks 5 to 8, Weeks 9 to 12, and Weeks 13 to 16 as repeated measure response variable and α4-integrin saturation level at Weeks 4, 8, 12, and 16 as the explanatory variable. A logit link function with a binomial underlying distribution was used. An exchangeable working correlation matrix between time points was also assumed. Predicted values of μ (probability of relapse occurrence) given saturation levels were derived from the model with a robust estimate of variance.

For patients who experienced relapse, their response values at all subsequent time intervals were imputed as “Y”.

### Trough α4-Integrin Saturation Level by Dosing Regimen

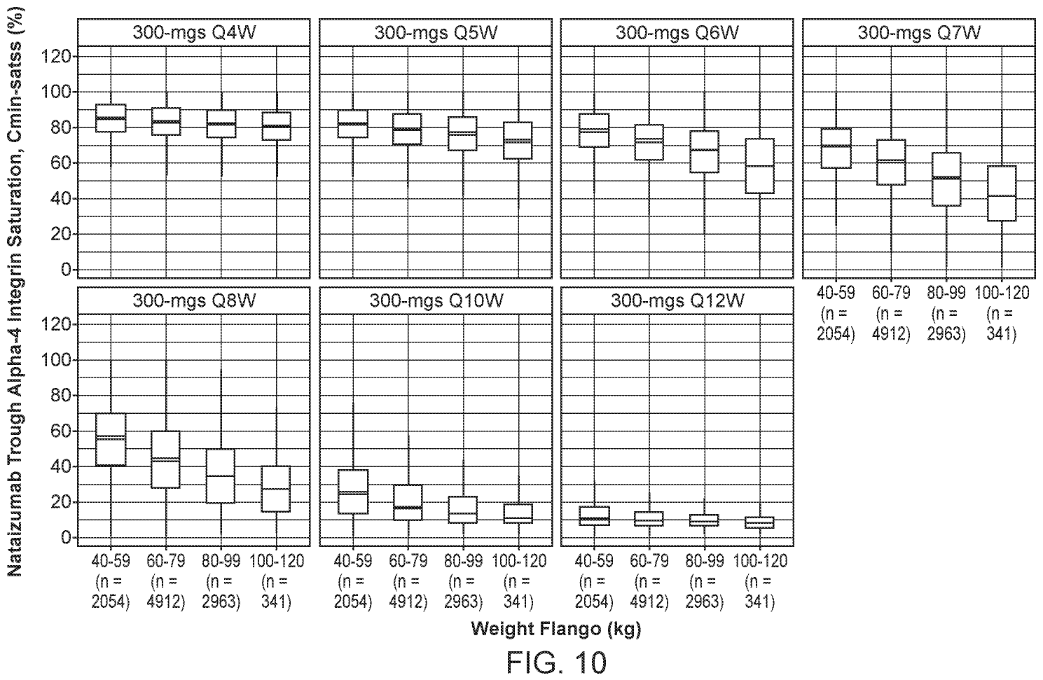

The trough α4-integrin saturation at Week 52 for each of the following dosing regimens: 300 mg Q4W, Q5W, Q6W, Q7W, Q8W, Q10W, and Q12W, were previously simulated from the PK-α4-integrin model by body weight categories (40 to <60 kg, 60 to <80 kg, 80 to <100 kg, and 100 to <120 kg), with 10,000 patients per dosing regimen group. The resulting distribution (Table 26 and FIG. 10) was used to draw random samples of steady-state trough saturation levels for various simulated populations.

TABLE 26
Simulated Natalizumab mean (95% prediction interval), trough alpha-4
integrin saturation (Cmin-sat, SS) at 52 weeks categorized by varying weight ranges

| | Trough Alpha-4 Integrin Saturation (%) | | | |
|---|---|---|---|---|
| | Weight Range (kg) | | | |
| Dosing | 40-59 | 60-79 | 80-99 | 100-120 |
| Regimen | (n = 2054) | (n = 4912) | (n = 2693) | (n = 341) |
| 300-mgs (Q4W) | 85.1 (65.3, 100) | 83.3 (62.1, 100) | 81.8 (59.4, 100) | 80.3 (53.6, 100) |
| 300-mgs (Q5W) | 82.5 (60.2, 100) | 78.8 (53.0, 100) | 76.0 (47.5, 100) | 72.0 (38.6, 99.1) |
| 300-mgs (Q6W) | 77.6 (49.0, 100) | 71.2 (34.4, 100) | 65.6 (23.6, 96.7) | 58.1 (15.5, 94.5) |
| 300-mgs (Q7W) | 67.9 (29.1, 98.0) | 59.7 (16.4, 94.8) | 50.5 (9.00, 88.6) | 42.2 (7.50, 81.5) |
| 300-mgs (Q8W) | 55.0 (12.9, 91.7) | 43.9 (7.50, 85.1) | 35.6 (5.60, 77.6) | 29.8 (3.90, 76.4) |
| 300-mgs (Q10W) | 27.2 (4.10, 66.6) | 20.6 (3.10, 55.4) | 16.7 (2.40, 46.1) | 14.5 (2.30, 40.1) |
| 300-mgs (Q12W) | 13.5 (2.00, 39.4) | 11.4 (2.00, 31.0) | 10.2 (1.40, 27.0) | 9.60 (1.90, 24.8) |

### Simulations for Efficacy by Dose Regimen

The proportion of patients with Gd+ lesions, the mean number of Gd+ lesions, and the cumulative probability of relapse in a given patient population were simulated following the 2 steps below.

### Step 1:

For a given population of size N, with a given body weight distribution, a random sample of N patients was drawn from the trough α4-integrin saturation level distribution described above by dosing regimen and body weight. A truncated normal distribution (truncated at level 0% and 100%) was assumed. It was assumed that the previous simulation results of trough α4-integrin saturation level at Week 52 represents a general steady-state level.

### Step 2:

For each simulated patient, the predicted probability or predicted mean number of lesions and the variance of the predicated value were derived from each model (Models 1, 2, and 3 above) based on the trough α4-integrin saturation level from Step 1 above. The binary response (Yes/No) of the occurrence of lesion (or relapse) or the number of lesions for the individual simulated patient was then randomly drawn from the binomial or negative binomial distribution with the predicted value and variance.

The above steps were repeated 10,000 times (10,000 simulations) for each dose regimen, and each patient population was considered. The GEE [Zeger 1988] model building was performed using SAS (version 9.4) GENMOD procedure. The random sampling for simulations was performed using SAS random number generating functions RANNOR, RANBIN, and RAND.

### Results

### Model 1

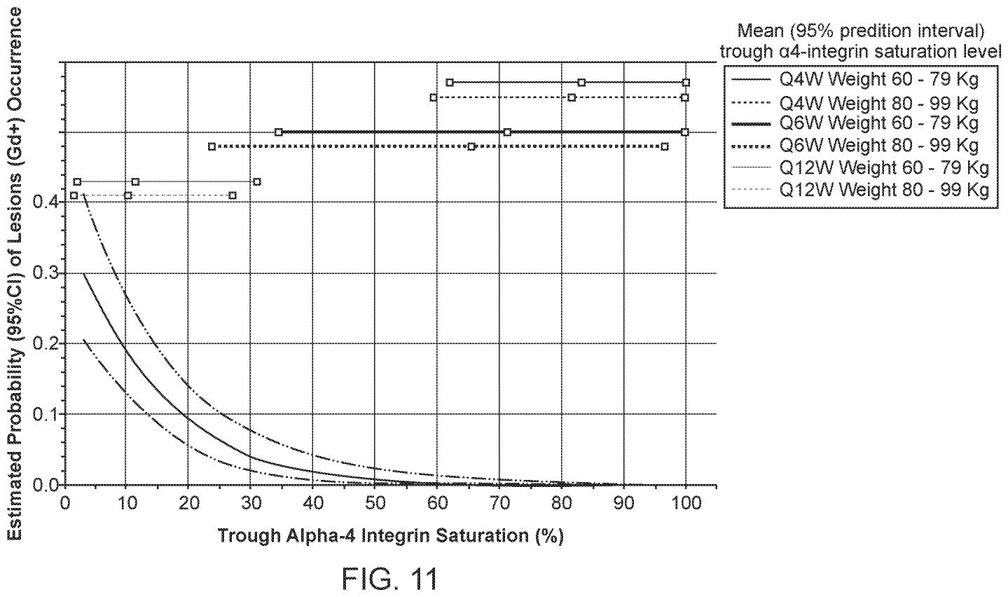

The estimates(±standard error [SE]) for the model parameter were the following: β0=−0.59±0.28 and β1=−0.09±0.01 (p<0.0001) for the logit response. The resulting fitted curve of the probability of Gd+ lesion occurrence with 95% confidence limits is provided in FIG. 11. To illustrate the distribution of the α4-integrin saturation level, the mean and 95% prediction interval are also plotted in FIG. 11 for dose regimens Q4W, Q6W, and Q12W.

The results show that in the expected trough saturation range for the Q4W dose regimen, the probability of lesion occurrence is very low but sharply rises with the expected range for Q12W.

The mean trough expected saturation level for the Q6W dose regimen appears to be in the region of low probability of lesion occurrence, however, with higher variability compared with that of Q4W and Q12W.

### Model 2

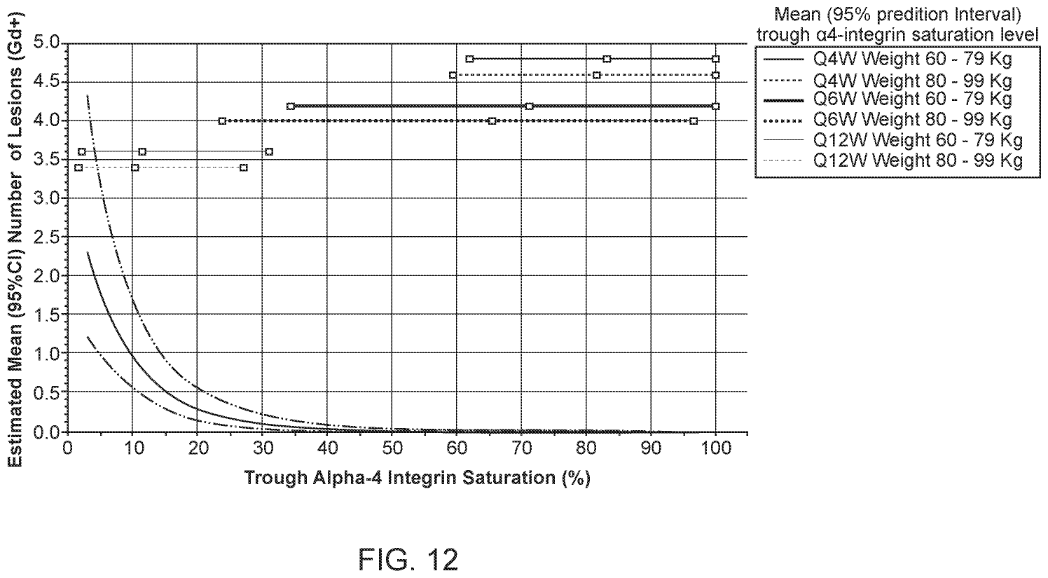

The estimates(±SE) for the model parameter were the following: β0=1.22±0.36 and β1=−0.13±0.02 (p<0.0001) for the log response. The resulting fitted curve of the mean number of Gd+ lesions with 95% confidence limits is provided in FIG. 12.

### Model 3

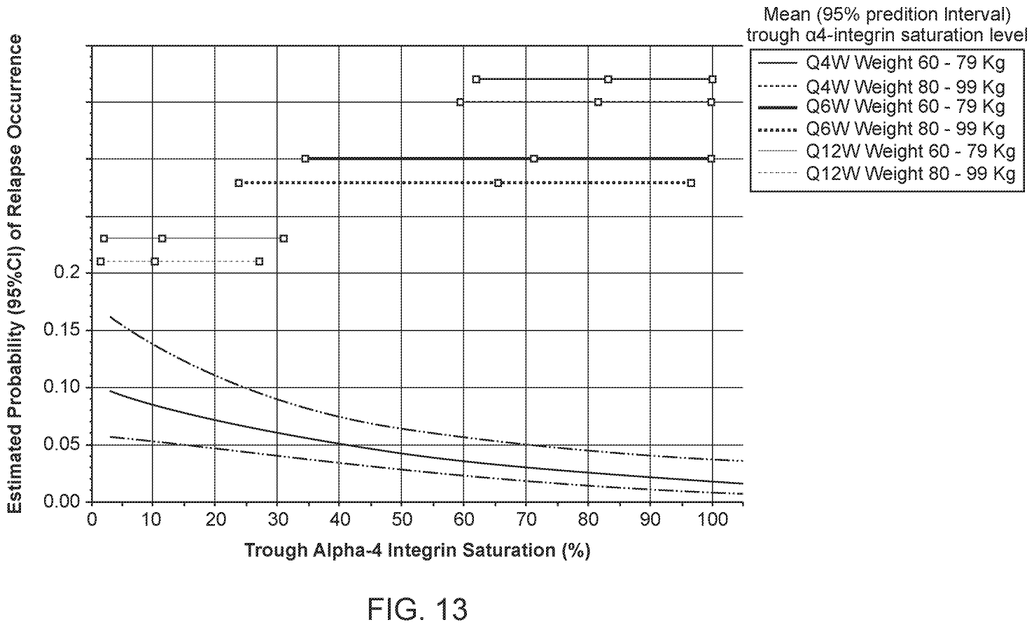

The estimates(±SE) for the model parameter were the following: β0=−2.18±0.31 and β1=−0.02±0.01 (p<0.0001) for the logit response. The resulting fitted curve of the probability of relapse occurrence with 95% confidence limits is provided in FIG. 13. The probability of relapse occurrence rises at a slower rate with decreasing saturation level, in contrast to the probability of lesion occurrence, and remains above zero even at high saturation level, suggesting that clinical relapse may be a more complex manifestation of disease activity than MRI lesion occurrence.

### Simulation of Dose Regimen Q4W and Q12W Outcomes of REFINE Study Population

Study 101MS206 (REFINE) was a prospective randomized study to evaluate various dosing regimens of natalizumab in patients who also have been receiving natalizumab at the standard doing regimen for at least 12 months with no MS relapse. REFINE study data on 300 mg IV Q4W and Q12W were used as a validation data set of the simulation results from the models.

The sample size and body weight distribution of the 300 mg IV Q4W and Q12W groups in the study were the basis for simulating the proportion of patients with Gd+ lesions, mean number of lesions, and cumulative probability of relapse in the study. A study population of 51 and 45 patients was simulated 10,000 times each for the Q4W and Q12W dose regimens, respectively. As more than 20% of the patients in the IV Q12W group in the study received rescue treatment after Week 24, the comparisons of simulation results and the actual observed results were restricted to the data up to Week 24. Within the range of variability, the simulated probability of Gd+ lesion occurrence, mean number of Gd+ lesions, and cumulative probability of relapse, using models built from RESTORE data, was similar to the observed results in the REFINE study (Table 27).

TABLE 27
Stimulation Results Based on REFINE Study Population Versus Observed
Study Results

| REFINE Population | Natalizumab 300 mg IV | |
|---|---|---|
| N(%) | Q4W | Q12W |
| Overall¹ | 51 | 45 |
| Weight Range (kg) | | |
| 40-59 | 15 (29.4%) | 11 (24.4%) |
| 60-79 | 25 (49.0%) | 24 (53.3%) |
| 80-99 | 10 (19.6%) | 9 (20.0%) |
| 100-120 | 1 (2.0%) | 1 (2.2%) |

Proportion of Patients with Gd+ Lesions at Week 24

| | | |
|---|---|---|
| Mean (5^{th} percentile, 95^{th} percentile) of | 0.1% (0%, 2.0%) | 18.9% (8.9%, 28.9%) |
| 10,000 Simulations From Model 1 | | |
| Observed Proportion if REFINE (95% CI) | 2.0% (0%, 10.5%) | 24.4% (12.9%, 39.5%) |

Mean Number of Gd+ Lesions at Week 24

| | | |
|---|---|---|
| Mean (5^{th} percentile, 95^{th} percentile) of | 0.0% (0.0%, 0.0%) | 1.26% (0.84%, 1.73%) |
| 10,000 Simulations From Model 2 | | |
| Observed Proportion if REFINE (95% CI) | 0.16% (0%, 0.47%) | 1.22 (0%, 2.82%) |

Cumulative Probability of Relapse Occurrence up to Week 24

| | | |
|---|---|---|
| Mean (5^{th} percentile, 95^{th} percentile) of | 3.7% (0.0%, 7.8%) | 12.7% (4.4%, 22.2%) |
| 10,000 Simulations From Model 2 | | |
| Observed Proportion if REFINE (95% CI) | 5.8% (0.0%, 12.2%) | 10.9% (1.9%,19.9%) |

CI = confidence interval;
Gd+ = gadolinium-enhancing;
IV = intravenous;
mITT = modified intent-to-treat;
Q4W = every 4 weeks;
Q12W = every 12 weeks.
¹Number of patients in mITT population with baseline body weight data available.

It is noted that the simulated results for Q4W dose regimen appear to be consistently lower than the actual observed results, although all were within the 95% confidence interval of the observed results. The REFINE study therefore cross-validates the models described herein.

### Simulation of Efficacy Outcomes for Dose Regimen 300 mg Q4W, QSW, Q6W, Q7W, Q8W, Q10W, and Q12W by Body Weight Category

Tables 28-30 provide the simulated outcomes by dose regimen and body weight category of the proportion of patients with Gd+ lesions, the mean number of Gd+ lesions, and the cumulative probability of relapse at Week 48 in populations of 500 patients each. The efficacy of natalizumab decreased with increasingly longer dosing intervals, as well as with body weight, in the simulated scenarios. However, the efficacy outcomes of Q5W and Q6W were close to that with the Q4W dosing regimen. The loss of efficacy appears to accelerate from dosing interval Q8W onward.

TABLE 28
Simulated Proportion (%) of Patients with Gd+ Lesions at
Week 48 by Body Weight Category

| | Proportion (%) of Patients with Gd+ Lesions at Week 48 | | | |
|---|---|---|---|---|
| | Mean (5^{th} percentile, 95^{th} percentile) of 10,000 Simulated | | | |
| | Populations of N = 500 | | | |
| | Weight Range (kg) | | | |
| Dosing | 40-59 | 60-79 | 80-99 | 100-120 |
| Regimen | (n = 500) | (n = 500) | (n = 500) | (n = 500) |
| 300-mgs (Q4W) | 0.1 (0.0, 0.2) | 0.1 (0.0, 0.4) | 0.1 (0.0, 0.4) | 0.1 (0.0, 0.4) |
| 300-mgs (Q5W) | 0.1 (0.0, 0.4) | 0.2 (0.0, 0.6) | 0.2 (0.0, 0.6) | 0. (0.0, 0.8) |
| 300-mgs (Q6W) | 0.2 (0.0, 0.6) | 0.5 (0.0, 1.0) | 1.0 (0.4, 1.8) | 1.8 (0.8, 2.8) |
| 300-mgs (Q7W) | 0.7 (0.2, 1.4) | 1.6 (0.8,2.6) | 2.8 (1.6, 4.0) | 3.8 (2.4, 5.2) |
| 300-mgs (Q8W) | 2.1 (1.2, 3.2) | 4.2 (2.8, 5.8) | 6.9 (5.0, 8.8) | 9.9 (7.8, 12.2) |
| 300-mgs (Q10W) | 10.1 (8.0, 12.4) | 13.2 (10.8, 15.6) | 15.2 (12.6, 18.0) | 16.6 (14.0, 19.4) |
| 300-mgs (Q12W) | 17.4 (14.6, 20.2) | 19.0 (16.2, 21.8) | 20.1 (17.2, 23.0) | 20.7 (17.8, 23.6) |

Gd+ = gadolinium-enhancing;
Q4W = every 4 weeks;
Q5W = every 5 weeks;
Q6W = every 6 weeks;
Q7W = every 7 weeks;
Q8W = every 8 weeks;
Q10W = every 10 weeks;
Q12W = every 12 weeks.

TABLE 29
Simulated Mean Number of Gd+ Lesions at Week 48 by Body Weight
Category

| | Mean Number of Gd+ Lesions at Week 48 | | | |
|---|---|---|---|---|
| | Mean (5^{th} percentile, 95^{th} percentile) of 10,000 Simulated | | | |
| | Populations of N = 500 | | | |
| | Weight Range (kg) | | | |
| Dosing | 40-59 | 60-79 | 80-99 | 100-120 |
| Regimen | (n = 500) | (n = 500) | (n = 500) | (n = 500) |
| 300-mgs (Q4W) | 0.00 (0.00, 0.00) | 0.00 (0.00, 0.00) | 0.00 (0.00, 0.00) | 0.00 (0.00, 0.00) |
| 300-mgs (Q5W) | 0.00 (0.00, 0.00) | 0.00 (0.00, 0.00) | 0.00 (0.00, 0.01) | 0.00 (0.00, 0.01) |
| 300-mgs (Q6W) | 0.00 (0.00, 0.01) | 0.01 (0.00, 0.02) | 0.03 (0.01, 0.06) | 0.06 (0.03, 0.09) |
| 300-mgs (Q7W) | 0.01 (0.00, 0.03) | 0.06 (0.03, 0.09) | 0.11 (0.07, 0.16) | 0.15 (0.10, 0.20) |
| 300-mgs (Q8W) | 0.08 (0.00, 0.00) | 0.20 (0.14, 0.26) | 0.39 (0.31, 0.48) | 0.65 (0.55, 0.76) |
| 300-mgs | 0.63 (0.53, 0.074) | 0.86 (0.75, 0.98) | 1.00 (0.88, 1.13) | 1.09 (0.97, 1.22) |
| (Q10W) | | | | |
| 300-mgs | 1.18 (1.05, 1.31) | 1.26 (1.13, 1.39) | 1.33 (1.21, 1.47) | 1.38 (1.25, 1.50) |
| (Q12W) | | | | |

Gd+ = gadolinium-enhancing;
Q4W = every 4 weeks;
Q5W = every 5 weeks;
Q6W = every 6 weeks;
Q7W = every 7 weeks;
Q8W = every 8 weeks;
Q10W = every 10 weeks;
Q12W = every 12 weeks.

TABLE 30
Stimulated Cumulative Probability of Relapse at Week 48 by Body Weight
Category

| | Cumulative Probability (%) of Relapse at Week 48 | | | |
|---|---|---|---|---|
| | Mean (5^{th} percentile, 95^{th} percentile) of 10, 000 | | | |
| | Simulated Populations of N = 500 | | | |
| | Weight Range (kg) | | | |
| Dosing | 40-59 | 60-79 | 80-99 | 100-120 |
| Regimen | (n = 500) | (n = 500) | (n = 500) | (n = 500) |
| 300 mg (Q4W) | 7.3 (5.4, 9.2) | 7.5 (5.6, 9.4) | 7.7 (5.8, 9.8) | 8.0 (6.0, 10.0) |
| 300 mg (Q5W) | 7.6 (5.8, 9.6) | 8.2 (6.2, 10.2) | 8.6 (6.6, 10.8) | 9.4 (7.2, 11.6) |
| 300 mg (Q6W) | 8.4 (6.4, 10.4) | 9.6 (7.4, 11.8) | 10.7 (8.6, 13.0) | 12.2 (9.8, 14.6) |
| 300 mg (Q7W) | 10.2 (8.0, 12.4) | 11.9 (9.6, 14.4) | 13.8 (11.4, 16.4) | 15.7 (13.0, 18.4) |
| 300 mg (Q8W) | 12.8 (10.4, 15.4) | 15.5 (12.8, 18.2) | 17.8 (15.0, 20.6) | 19.5 (16.6, 22.4) |
| 300 mg (Q10W) | 20.2 (17.2, 23.2) | 22.2 (19.2, 25.4) | 23.6 (20.6, 26.8) | 24.4 (21.4, 27.6) |
| 300 mg (Q12W) | 24.8 (21.6, 28.0) | 25.7 (22.4, 29.0) | 26.2 (23.0, 29.6) | 26.5 (23.2, 29.8) |

Q4W = every 4 weeks;
Q5W = every 5 weeks;
Q6W = every 6 weeks;
Q7W = every 7 weeks;
Q8W = every 8 weeks;
Q10W = every 10 weeks;
Q12W = every 12 weeks.

### Conclusions

The models characterizing the relationship between efficacy outcomes and a4-integrin saturation level were developed using data from Study 101MS205 (RESTORE). These models were then used to simulate the efficacy outcomes of Study 101MS206 (REFINE) as a validation. The simulated results were generally similar to the actual observed results of REFINE. The models then were used to simulate the proportion of patients with Gd+ lesions, the mean number of Gd+ lesions, and the cumulative probability of relapse at Week 48 for various dosing regimens by body weight category. The efficacy is maintained at high levels across all body weights with Q4W and Q5W dosing. It starts to decrease more appreciably in the 2 higher weight categories with Q6W and is progressively worsened with less frequent dosing. The PK of Tysabri is characterized by a mean±SD half-life of 16±4 days. Thus, after a switch to the extended dosing interval, stable trough concentrations will be reached after about 15-24 weeks.

Natalizumab PK can exhibit linear and nonlinear elimination. Nonlinearities in PK can cause changes in integrin binding that are disproportionate to concentrations and lead to higher variability in a4-integrin saturation levels in some cases. In some embodiments, the simulation results are interpreted with this limitation of uneven variability taken into consideration. As modelling was based on RESTORE data up to rescue (Week 16), no patient had more than 1 relapse episode, and thus, in some embodiments, the model can only estimate the cumulative probability of relapse occurrence instead of ARR.

### Scientific Discussion

The analysis of the TOUCH database and the PML database conclusively demonstrates that EID treatment is associated with a lower risk of PML than SID treatment in anti-JCV antibody-positive patients.

Effectiveness data have not been included in some cases. In some cases, the lack of included effectiveness data in a provided dataset precludes assessment of the comparable benefit-risk of EID versus SID. In some embodiments, the results from updated analysis of the TOUCH data, e.g., in combination with prospective studies described herein, are generalizable to patients in the U.S. population, including patients that have not previously been treated with natalizumab. In some embodiments, the results from updated analysis of the TOUCH data, e.g., in combination with prospective studies described herein, are generalizable to patients in the U.S. and EU populations, including patients that have not previously been treated with natalizumab. In some embodiments, the results from updated analysis of the TOUCH data, e.g., in combination with prospective studies described herein, are generalizable to patients in need of treatment with an α4-integrin inhibitor, including patients that have not previously been treated with natalizumab.

Hazards of PML in both the EID and SID group were compared using Cox regression models adjusted for age, gender, prior use of IS therapy, initiation calendar year, and number of infusions. These demographic characteristics were balanced across both groups; however, no information has been provided regarding the body weight and distribution of body weight, although the pharmacokinetic characteristics of natalizumab and Modeling and Simulation studies have shown that body weight can be a factor with respect to efficacy (see part C). In some embodiments, the MAH provides a detailed subgroup analysis of the provided retrospective analysis of EID versus SID regarding body weight. Body weight quartiles as well as different body weight cut-offs may be addressed to better evaluate the PML risk for specific subgroups.

### Proposal to Further Investigate the Efficacy and Safety in Terms of PML Risk Reduction of EID Relative to SID;

In general the conduct of the clinical study to further investigate whether the effectiveness of natalizumab is maintained with EID treatment and to better inform on the EID benefit-risk in special patient populations is endorsed. The MAH provides a comprehensive proposal:

The PK of Tysabri is characterized by a mean±SD half-life of 16±4 days. Thus, after a switch to the extended dosing interval, stable trough concentrations will be reached after about 15-24 weeks. Consequently, the primary clinical endpoint of the planned study should be shifted towards the end and set to week 72.

As outlined in part A body weight is a factor with respect to efficacy and will be addressed in the planned Study (part B) which is highly supported. However, the proposed weight cut-off point of 90 kg is not completely supported due to the following considerations:
The majority of MS patients are female; only relatively few patients are expected to meet the >90 kg BW criterion.
Based on current modelling and simulation results, EID treatment (Q6W) may lead to low Ctrough and alpha integrin saturation levels in patients with a body weight at 80 kg and above.

Consequently, the selection of 90 kg for comparative analyses is considered too high and should be set to a clinically and statistically justified lower value. In addition, body weight should be included as a continuous variable in supplementary analyses regarding weight to better evaluate the explicit impact of weight on efficacy in the planned study.

Updated PK/PD Modelling Taking into Account Body Weight and Extended Dosing Intervals

Model-based conclusions of an updated the PK/PD model indicate that the Q6W dosing can be supported without major loss of effectiveness/efficacy, however there remains a moderate degree of uncertainty.

In the context of updating the PK/PD modelling, three integrin saturation vs. efficacy outcome models have been established based on RESTORE data only.

Generalized estimating equations were used to compare the integrin saturation level with the three efficacy parameters probability of lesion occurrence, mean lesion number, and probability of relapse occurrence by trough integrin saturation level. For some patients, response values such as the number of lesions observed have been imputed regardless of the actual scan results.

Despite the estimates±standard error for the two model parameter (β0, β1) for each of the three models, no further evaluation of these models have been provided (observed data not shown, no detailed modelling report, no clear rationale with respect to data/model selection for model building and model validation, no information about impact and sensitivity regarding imputed observed values). The applicant is asked to provide detailed information to better assess the model appropriateness and uncertainties.

For validation of the established models, data from the REFINE study have been used, which is considered acceptable. Based on integrin saturation data (Q4W, Q12W) and efficacy data, model-based predictions indicated a trend of underestimation of efficacy outcome in comparison to observed values.

Critial point during model-based simulation is the Xij-Matrix that reflects integrin saturation and thus represents the link to efficacy and PD parameters.

Further simulations of Ctroughs and integrin saturation for various regimens (Q4W, Q5W, Q6W, . . . , Q12W) were based on previous PK/PD model (Muralidharan et al. 2016) which is deemed acceptable.

The applicant is asked to indicate RESTORE-related saturation data versus natalizumab serum concentration in connection with observations recruited from all other relevant studies.

Further simulations of efficacy outcome based on RESTORE modelling suggest that efficacy is maintained across all body weights with Q4W and Q5W dosing. It starts to decrease more appreciably in the two higher weight categories (>80 kg) with Q6W and is progressively worsened with less frequent dosing.

The MAH is asked to simulate the PK (Ctrough) and integrin saturation over the complete study duration, taking the level of natalizumab before switching to extended dosing into account. Like previous reporting, results should be provided categorised by varying weight ranges from 40 kg to 120 kg. These simulation may provide further insight in where to set a cut-off point for comparative analyses regarding weight in the planned Study (see part B).

An appropriate primary analysis of body weight during the prospective study is deemed necessary to provide evidence that 300 mg Q6W is acceptable for all subjects or if further adjustment of posology for some subgroups is warranted.

Notably, for simulation results regarding Cavg, Ctrough and integrin saturation the MAH referred to previous PSUR response. However, this comprises IV simulations only; simulations for the various regimens given SC were not provided. SC data and SC mode of application have been completely excluded from the MAH's considerations. SC program Q6W (adjusted for F) could be favorable with respect to higher Ctrough/saturation at week 6 at lower or similar Cavg. In this line, no further time course plots after switching to extended dosing have been provided.

### Overall Conclusion

### Part A (TOUCH):

From the provided analysis it can be concluded that each EID group was associated with a clinically and statistically significantly lower risk of PML compared with the SID group in anti-JCV antibody-positive patients (and patients without history of IS treatment). In some embodiments, a stratified analysis regarding body weight is provided to better evaluate the risk in certain subgroups and to compare, contrast, or harmonize these results with results from the planned study.

### Part B (Phase 3b Study):

In general, the conduct of the clinical study to further investigate whether the effectiveness of natalizumab is maintained with EID treatment and to better inform on the EID benefit-risk in special patient populations is endorsed. However, the MAH's proposal needs to be reconsidered and amended regarding several aspects. The definition and justification of the margin may be discussed in a EMA Scientific Advice procedure.

### Part C (PK/PD Analyses):

Modelling results indicate a decrease in efficacy with increase in body weight and duration of dosing interval. Further simulations of efficacy outcome based on RESTORE modelling suggest that efficacy is maintained across all body weights with Q4W and Q5W dosing. It starts to decrease more appreciably in the two higher weight categories (>80 kg) with Q6W and is progressively worsened with less frequent dosing. Model-based conclusions indicate that the Q6W can be supported for patients with a body weight below 80 kg, however there remains a moderate degree of uncertainty. The MAH is asked to conduct some additional analyses and simulations to increase model-based evidence and to better assess the quantitative model predictability.

### Regulatory Impact

Considering the large PML risk minigation effect of EID and the result of the updated pk/pd modelling that extended interval dosing does not have a major effect on efficacy (e.g., in patients with a body weight <80 kg) there is a need for a regulatory update and communication of these new data. The MAH is asked to make a proposal. This is deemed necessary as the final results of the proposed CT are expected in Q2 2021 and waiting for the CT study results will not be acceptable based on the current knowledge.

### Part A (TOUCH):

1. The MAH is asked to provide detailed subgroup analyses of retrospective analysis of EID versus SID regarding body weight. Body weight quartiles as well as other clinically meaningful body weight cut-offs may be addressed. Analyses from TOUCH should be updated as more data and data on body weight are accrued. This analysis should be used to further justify the cut-off used for randomization in the planned Phase 3b study.
   Part B (planned Phase 3b study):
2. The primary clinical endpoint of the planned study should be shifted towards the end and set to week 72.
3. Stratification by body weight with a cut-off of 90 kg is considered not optimal. First, the subgroup >90 kg is expected to be rather small and hence, randomization will most likely not be well balanced. Second, the justification of 90 kg as cutoff is not comprehensible from other clinical trials including TOUCH data. The MAH is asked to reconsider the cutoff for body weight in the Phase 3b and provide a justification for the cutoff. In addition, body weight should be included as a continuous variable in supplementary analyses regarding weight to better evaluate the explicit impact of weight on efficacy in the planned study.
4. Currently, the primary statistical analysis is only based on confidence intervals with no clearly pre-specified success criterion. However, it should be based on a non-inferiority test. This includes the pre-specification and justification of the non-inferiority margin based on clinical and statistical grounds (see Guideline on the choice of the non-inferiority margin; EMEA/CPMP/EWP/2158/99). This margin is preferably to be defined for the difference in rates rather than the ratio and must be based on data for the requested primary endpoint.
5. The MAH should note that testing for superiority of SID over EID at interim does not necessarily preclude a positive study as an endpoint can be significantly less effective and non-inferior at the same time, depending only on the width of the confidence interval and the width of the margin. An appropriate futility analysis should hence be prespecified.
6. The MAH should provide more details on the handling of intercurrent events (initiation of rescue medication, switch in treatment regimen due to AEs or lack of efficacy, treatment discontinuation due to AEs, treatment discontinuation due to lack of efficacy, etc.) often falsely labelled as missing data (compare ICH E9 (R1) Draft Addendum on estimands; EMA/CHMP/ICH/436221/2017) and treated inappropriately in the analysis. The MAH should detail the planned estimands, i.e., what is to be estimated, how these events are handeled in the primary analysis and which sensitivity or supplementary analyses are foreseen.
7. Currently, the stratification factors used for randomization and in the primary analysis are not well aligned. Duration of natalizumab exposure is used (amongst other factors) at time of randomization, while EDSS is used instead in the primary analysis. The guideline on adjustment for baseline covariates in clinical trials (EMA/CHMP/295050/2013) states that “stratification variables, if not solely used for administrative reasons, should usually be included as covariates or stratification variables in the primary analysis regardless of their prognostic value. Any mismatch of non-administrative covariates between stratification and adjustment in the primary analysis must be explained and justified.” The MAH should hence modify or justifiy the applied stratification variables. Furthermore, it should be clarified how EDSS is treated in the primary analysis model (i.e., as linear covariate or as categorical effect) if kept in the model.
8. Pre-planned subgroup analyses should be foreseen in the protocol. These should especially include subgroup analyses for body weight (with pre-specified cutoffs). Results for both, efficacy and safety should be presented in these subgroups to allow an appropriate benefit-risk discussion.
9. The definition and justification of the study design including non-inferiority margin should be discussed in a EMA Scientific Advice procedure. Both, statistical analysis methods and sample size planning should reflect these considerations appropriately.

### Part C (PK/PD Analyses):

10. Despite the estimates ±standard error for the two model parameter (β0, β1) for each of the three models, no further evaluation of these models have been provided (observed data not shown, no detailed modelling report, no clear rationale with respect to data/model selection for model building and model validation, no information about impact and sensitivity regarding imputed observed values). The applicant is asked to report detailed information according to respective guidelines to better assess the model appropriateness and uncertainties.
11. Further simulations of Ctroughs and integrin saturation for various regimens (Q4W, Q5W, Q6W, . . . , Q12W) were based on previous PK/PD model (Muralidharan et al. 2016) which is deemed acceptable. The applicant is asked to indicate RESTORE-related saturation data versus natalizumab serum concentration in connection with observations recruited from all other relevant studies.
12. The MAH is asked to simulate the PK (Ctrough) and integrin saturation over the complete study duration, taking the level of natalizumab before switching to extended dosing into account. Like previous reporting, results should be provided categorised by meaningful weight ranges from 40 kg to 120 kg.

### Regulatory Consequences

13. Considering the large PML risk mitigation effect of EID and the result of the updated pk/pd modelling that extended dosing does not have a major effect on efficacy (at least in patients with a body weight <80 kg) there is a need for a regulatory update and communication of these new data. Therefore, the MAH is requested to provide a proposal for an updated SmPC as well as a draft Dear Healthcare Provider Communication (DHPC) for review by PRAC. The changes to the product information should be agreed with PRAC during the ongoing procedure LEG 066, and should thereafter be implemented as part of a subsequent type IB variation.

### Example 7: Reduction in Progressive Multifocal Leukoencephalopathy Risk with Natalizumab Extended Interval Dosing

### Abstract

### Objective

To use the large dataset from the Tysabri Outreach: Unified Commitment to Health (TOUCH®) program to compare progressive multifocal leukoencephalopathy (PML) risk with natalizumab extended interval dosing vs standard interval dosing in multiple sclerosis patients.

### Methods

This retrospective cohort study included anti-JC virus antibody positive patients (N=35,521) with dosing intervals >3 weeks and <12 weeks in the TOUCH database as of Jun. 1, 2017. The effect of EID on PML risk was evaluated with 3 planned analyses. Cumulative PML risk in EID and SID cohorts was estimated using Kaplan-Meier methods stratified by prior immunosuppressant use. Risk of PML was analyzed by Cox regression adjusted for age, sex, prior immunosuppressants, time since natalizumab initiation, and cumulative number of infusions.

### Results

This study included 35,521 patients (primary analysis: 1988 EID, 13,132 SID; secondary analysis: 3331 EID, 15,424 SID; tertiary analysis: 815 EID, 23,168 SID). Mean average dosing intervals were 35.0-43.0 days and 29.8-30.5 days for the EID and SID cohorts, respectively. Hazard ratios (95% CIs) of PML risk for EID vs SID were 0.06 (0.01-0.22; p<0.001) for the primary analysis and 0.12 (0.05-0.29; p<0.001) for the secondary analysis. Relative risk reductions were 94% and 88% in favor of EID for the primary and secondary analyses, respectively. There were no PML cases with EID in the tertiary analysis.

### Conclusions

Natalizumab EID is associated with clinically and statistically significantly lower PML risk than SID. Further studies are needed to evaluate EID efficacy.

### Introduction

Natalizumab, a monoclonal antibody directed against the α4-integrin cell adhesion molecule, is an efficacious treatment for relapsing forms of multiple sclerosis (MS), as demonstrated by randomized clinical trials^{1,2} and real-world data.^{3,4} The recommended treatment schedule (300 mg intravenous infusion every 4 weeks) was selected to provide >80% saturation of mononuclear cell α4β1-integrin receptors for approximately 1 month after administration.^{5,6} For patients previously exposed to JC virus (JCV), natalizumab treatment is associated with a risk of progressive multifocal leukoencephalopathy (PML).⁷ Established risk factors for PML in anti-JCV antibody positive patients include the level of anti-JCV antibodies in serum as assessed by anti-JCV antibody index, the use of immunosuppressant therapy prior to natalizumab initiation, and the duration of natalizumab treatment.^{8,9}

In real-world practice, treatment cessation, treatment interruptions, and deviations from recommended treatment schedules are not unusual. Several retrospective studies have investigated the effect of extended interval dosing (EID) schedules (infusion intervals >4 weeks) with the goal of maintaining natalizumab efficacy while reducing the risk of PML.^{10, 11} These studies, which are limited by non-randomized designs, small patient populations, and variable definitions of EID, nevertheless suggest that patients switching to natalizumab EID after a period of standard interval dosing (SID) continue to do well. However, because PML is a rare event, these studies did not have sufficient statistical power to assess whether EID is associated with risk reduction of PML relative to SID. Therefore, the safety of natalizumab EID with respect to PML risk is not fully known.

The Tysabri Outreach: Unified Commitment to Health (TOUCH®) program, a risk evaluation and mitigation strategy mandated by the US Food and Drug Administration,^{7,12} is designed to inform healthcare providers and patients about PML and its known risk factors; to warn against concurrent use of antineoplastic, immunosuppressant, or immunomodulatory agents; and to monitor patients for development of PML and other serious opportunistic infections during treatment. The TOUCH database captures all natalizumab infusion records, patient demographic information, prior immunosuppressant therapy, and anti-JCV antibody status data (since February 2012). It is the largest dataset in the world that can provide safety information associated with alternative dosing intervals of natalizumab.

Methods

Study design

This retrospective cohort study included data collected in the TOUCH program as of Jun. 1, 2017, and included all patients with a known positive anti-JCV antibody serostatus and a known status of prior immunosuppressant use. PML data up to Jun. 1, 2017, from Biogen's Tysabri Global Safety Database were also included in the study. Patients with a history of any interval >12 weeks (“dosing gap”) or <3 weeks (“overdose”) between 2 consecutive infusions were excluded. The 3 planned analyses and their respective EID and SID inclusion criteria were developed and finalized under conditions blinded to PML events.

### Primary Research Question

The objective of this study was to use the large, real-world TOUCH dataset to determine whether natalizumab EID was associated with a reduced PML risk compared with SID. Because there is no precise understanding of the mechanism whereby natalizumab causes PML or how dosing schedules might affect PML risk, 3 planned analyses, each with different EID inclusion criteria, were employed to evaluate both the impact and the potential mechanism of EID on PML risk.

Classification of Evidence

This study provides Class IV evidence that in patients with relapsing-remitting multiple sclerosis, natalizumab EID is associated with statistically and clinically significant reductions in PML risk compared with SID.

Data Collection

Patient data collected in TOUCH include demographic information, the date and dose of each natalizumab infusion, the date and results of anti-JCV antibody testing (since 2012) performed in the previous 12 months, and treatment with immunomodulatory/immunosuppressant therapies in the previous 6 months. The records of PML cases are captured and maintained in a separate pharmacovigilance database (the Tysabri Global Safety Database).

Planned Analyses and Inclusion Criteria

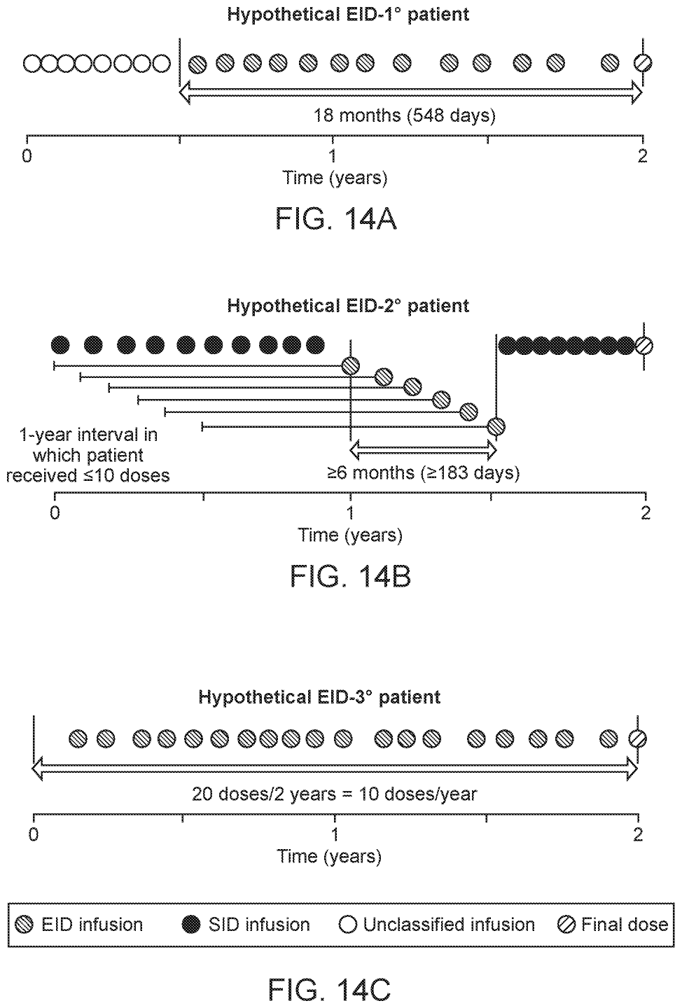

The TOUCH dataset demonstrates considerable variability in natalizumab dosing, whether intentional or unintentional, in US clinical practice. Furthermore, optimal EID infusion intervals and treatment duration are unknown. Therefore, 3 distinct analyses of EID vs SID were planned for this study. Each analysis employed different inclusion criteria (definitions) for EID and SID patients based on the number of doses received during specified time periods in order to test different hypotheses about the potential impact of EID on PML risk (FIGS. 14A-C). Patients could meet inclusion criteria for >1 analysis.

The primary analysis assessed PML risk associated with the last 18 months of recorded infusion history. Patients who had received ≤15 infusions in the last 18 months of treatment were included in the primary EID (EID-1°) analysis group; patients who had received >15 infusions in the last 18 months of treatment were included in the primary SID (SID-1°) analysis group.

The secondary analysis assessed the effect of any prolonged period of EID in the patient's infusion history on PML risk. For this analysis, individual infusions were categorized as EID or SID. An EID infusion was defined as any infusion preceded by ≤10 infusions in the prior 365 days. Patients receiving such EID infusions consecutively for ≥6 months were included in the secondary EID (EID-2°) analysis group. Similarly, an SID infusion was defined as any infusion preceded by >10 infusions in the prior 365 days, and patients receiving such infusions consecutively for ≥6 months were included in the secondary SID (SID-2°) analysis group. Patients with a history of both ≥6 months of EID-2° dosing and ≥6 months of SID-2° dosing were included in the EID-2° cohort only. Patients with >1 EID-2° regimen were excluded, increasing the analytical rigor.

The tertiary analysis assessed the effect of a dosing history consisting primarily of EID on PML risk. Patients who had received ≤10 infusions per year (annualized number of infusions) over their entire treatment history were included in the tertiary EID (EID-3°) analysis group; patients who had received >10 infusions per year were included in the tertiary SID (SID-3°) analysis group.

Two prespecified sensitivity analyses were performed. In the first, PML cases occurring before 2012 (prior to collection of anti-JCV antibody test results in TOUCH) were assumed to be anti-JCV antibody positive and added to the 3 planned analyses described above. In the second sensitivity analysis, alternative EID definitions of ≤13 infusions in the last 18 months and ≤9 infusions over any 12-month period were used for inclusion in the primary and secondary EID analysis groups, respectively. Alternative inclusion criteria for the tertiary analysis were not tested.

All analyses were performed on de-identified data collected in the TOUCH program with patient consent and on PML data collected via standard pharmacovigilance practices in order to monitor natalizumab safety as required by regulatory authorities. Additional informed consent was not required.

### Statistical Analysis

Demographic and treatment history data for the overall study population and for each EID analysis cohort were summarized by descriptive statistics. For the 3 planned analyses, time-to-event (PML occurrence) analyses using Kaplan-Meier estimates of cumulative risk were performed for the EID and SID cohorts. Time-to-event was based on time since initiation of natalizumab treatment. A log-rank test was performed to compare the time-to-event between the EID and SID cohorts. The conditional probability of PML in each exposure epoch (defined as a series of 12 infusions) was derived for the EID and SID cohorts using the life-table method stratified by prior immunosuppressant use. The PML hazard ratio (HR) in the EID and SID cohorts was estimated using a time-varying covariate Cox regression model adjusted for age, sex, calendar year of the start of natalizumab treatment, and prior immunosuppressant use (yes/no) as covariates and the cumulative number of infusions as the time-varying covariate.

For each analysis, the PML HR estimate (EID vs SID) and its 95% confidence interval (CI) from the Cox model were the primary basis of inference. Specifically, if the HR upper 95% CI limit was <1, the EID cohort would be considered to have a lower risk of PML than the SID cohort. If the HR point estimate was ≥0.9 and ≥1.1, the EID and SID cohorts would be considered to have similar risks. If the HR lower 95% CI limit was >1, the EID cohort would be considered to have greater risk. At the time of analysis plan specification, the anticipated study population sizes and expected number of PML events predicted approximately 85% power to detect a risk reduction ≥50% (i.e., a HR ≥0.5) as defined by the above rules of inference.

The statistical analysis plan was developed and finalized under conditions blinded to PML events. PML data from the Tysabri Global Safety Database were merged with TOUCH after the analysis plan was finalized.

Results

Patients

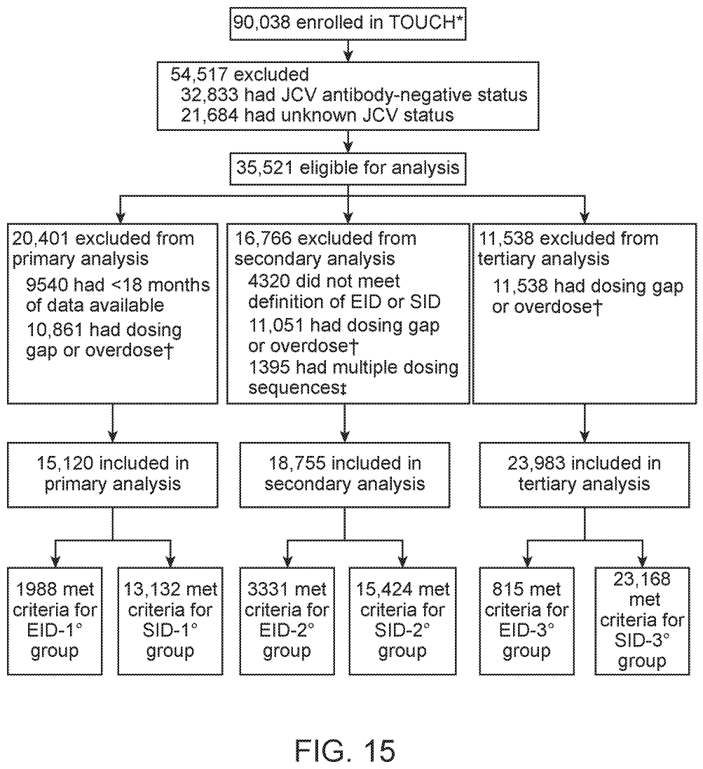

Of the 90,038 patients enrolled in TOUCH as of Jun. 1, 2017, 35,521 were anti-JCV antibody positive and eligible for this study (FIG. 15). After applying the prespecified EID and SID inclusion criteria, the study populations included 1988 EID and 13,132 SID patients in the primary analysis, 3331 EID and 15,424 SID patients in the secondary analysis, and 815 EID and 23,168 SID patients in the tertiary analysis. The most common reasons for patient exclusion were the presence of dosing gaps or overdoses in treatment history (primary, secondary, and tertiary analyses) and <18 months of available dosing data (primary analysis only).

The baseline demographics in the EID and SID groups were well balanced across the 3 analyses (Table 31). In all 3 analyses, EID patients had more natalizumab infusions and longer total duration of natalizumab treatment than SID patients. EID patients included in the primary analysis had received a median (range) of 37 (1-117) infusions before starting EID. In the secondary analysis (in which each infusion was defined as either EID or SID), EID-2° patients had received a median (range) of 25 (1-121) infusions before starting EID. For all 3 analyses, the average dosing interval (ADI) over the entire treatment duration was 35.0-43.0 days for EID patients and 29.8-30.5 days for SID patients.

TABLE 31
Baseline characteristics, natalizumab exposure, and ADIs

| | Primary analysis | | Secondary analysis | | Tertiary analysis | |
|---|---|---|---|---|---|---|
| | EID-1° | SID-1° | EID-2° | SID-2° | EID-3° | SID-3° |
| | group | group | group | group | group | group |
| Characteristic | (1988) | (13,132) | (3331) | (15,424) | (815) | (23,168) |
| Females, n (%)^{a} | 1376 | 8846 | 2293 | 10,239 | 539 | 15,636 |
| | (69) | (67) | (69) | (66) | (66) | (67) |
| Age at first | 42.9 | 44.0 | 43.0 | 43.9 | 42.0 | 43.9 |
| infusion, mean | (11.3) | (11.0) | (11.2) | (11.4) | (11.4) | (11.6) |
| (SD), y | | | | | | |
| Prior IS therapy, | 95 | 689 | 175 | 799 | 49 | 1310 |
| n (%)^{b} | (5) | (5) | (5) | (5) | (6) | (6) |
| Number of | 50 | 46 | 51 | 27 | 32 | 26 |
| natalizumab | (11, 132) | (17, 142) | (6, 137) | (7, 142) | (2, 103) | (1, 142) |
| infusions, | | | | | | |
| median (min, | | | | | | |
| max) | | | | | | |
| Duration of | 59 | 44 | 56 | 26 | 43 | 25 |
| natalizumab | (19, 130) | (19, 131) | (8, 131) | (7, 130) | (3, 129) | (1, 131) |
| treatment, | | | | | | |
| median (min, | | | | | | |
| max), mo | | | | | | |
| ADI, d | | | | | | |
| Mean (SD) | 36.7 | 30.0 | 35.0 | 29.8 | 43.0 | 30.5 |
| | (4.9) | (1.6) | (4.9) | (1.7) | (5.4) | (2.6) |
| Q1, Q3 | 33, 39 | 29, 31 | 32, 37 | 29, 31 | 39, 45 | 29, 31 |

Abbreviations: ADI = average dosing inerval (over entire treatment history); EID = extended interval dosing; IS = immunosuppressant; Q1 = first quartile; Q3 = third quartile; SID = standard interval dosing.
Refer to FIG. 14 and description for definitions of EID and SID in the primary, seconday, and tertiary analyses.
^{a}Information on patient sex was missing for <1% of patients in each group.
^{b}Information on prior IS therapy was missing for 4%-5% of patients in each group.

Risk Assessment

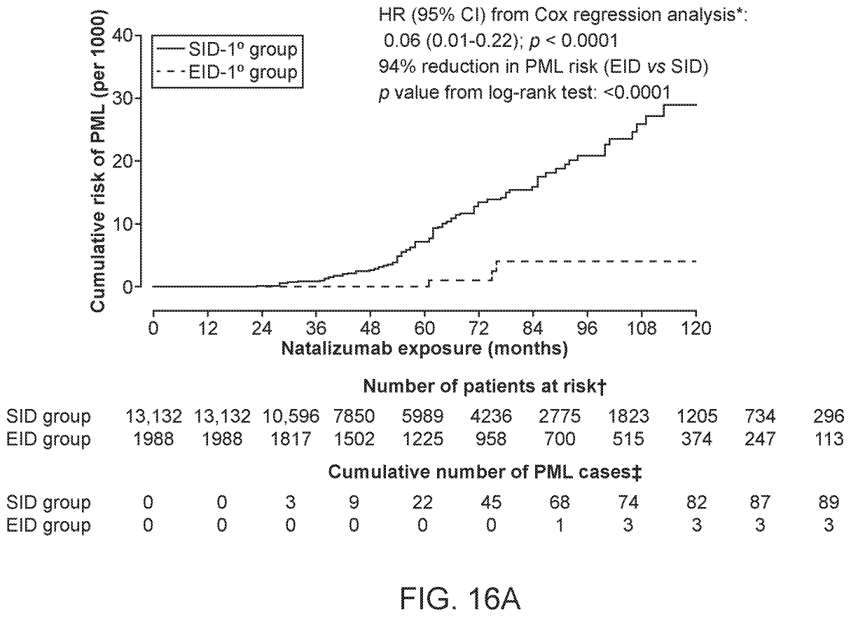

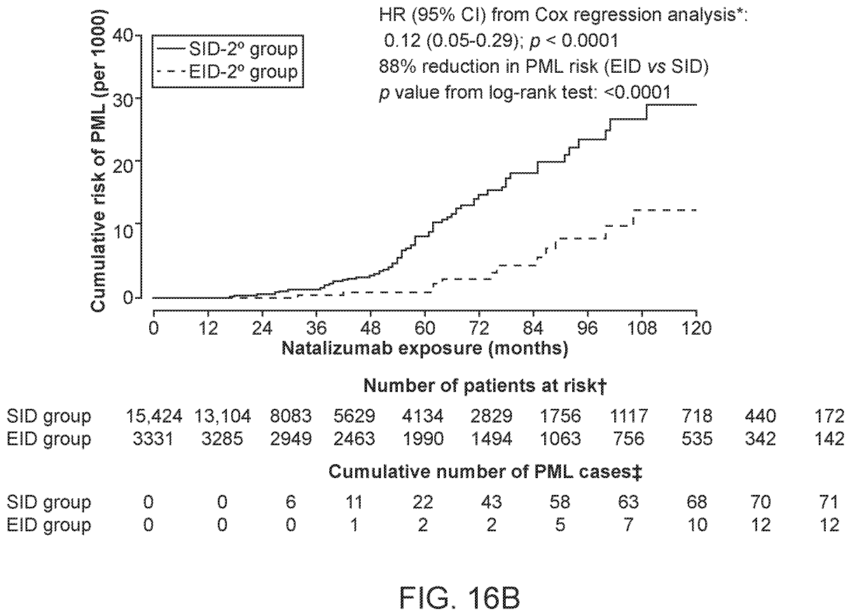

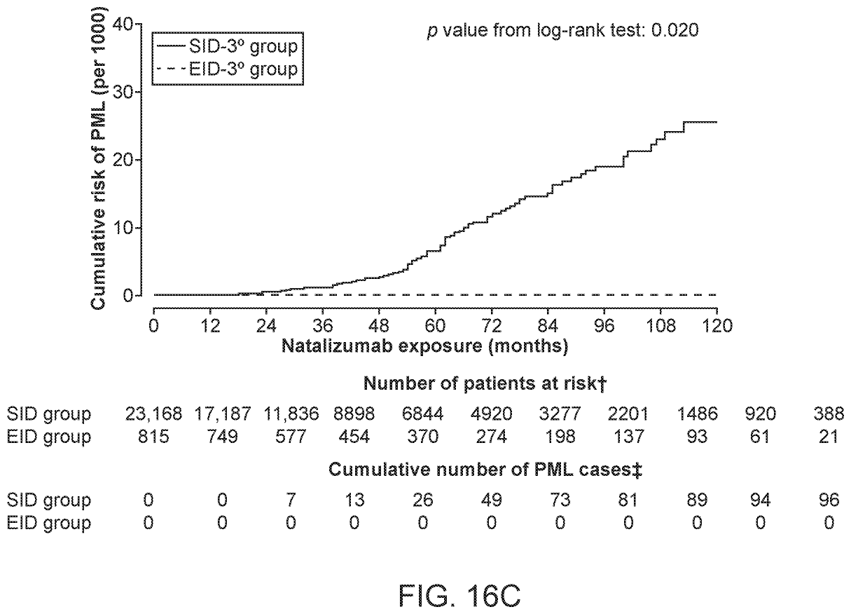

The Kaplan-Meier estimated cumulative risk of PML was significantly lower with EID than with SID (FIGS. 16A-C). In the primary and secondary analyses, cumulative risk appeared to separate after 24-36 months, with separation increasing at later time points. Cox regression analysis also identified significant reductions in PML risk with EID treatment in the primary and secondary analyses (both p<0.001; Table 32). The covariate-adjusted HR in the primary analysis was 0.06 (95% CI 0.01-0.22), corresponding to a relative risk reduction of 94% in EID-1° patients vs SID-1° patients. In the secondary analysis, the covariate-adjusted HR was 0.12 (95% CI 0.05-0.29), corresponding to a relative risk reduction of 88% in EID-2° patients vs SID-2° patients. As no PML cases were observed with EID in the tertiary analysis, the risk-reduction point estimate was 100% and the Cox regression model 95% CI was non-estimable.

TABLE 32
Impact of EID vs SID on PML risk in a Cox regression model
in the primary and secondary analyses^{a}

| | Primary analysis | | Secondary analysis | |
|---|---|---|---|---|
| Risk factor | HR (95% CI) | p value | HR (95% CI) | p value |
| Age | 1.00 (0.98-1.02) | 0.999 | 0.99 | 0.411 |
| | | | (0.97-1.01) | |
| Sex (male, female) | 1.05 (0.58-1.63) | 0.828 | 0.99 | 0.969 |
| | | | (0.63-1.57) | |
| Prior IS use | 2.92 (1.67-5.11) | <0.001 | 2.90 | <0.001 |
| (yes, no) | | | (1.60-5.27) | |
| Calendar year at | 0.99 (0.88-1.12) | 0.881 | 0.94 | 0.327 |
| the start of | | | (0.83-1.06) | |
| treatment | | | | |
| Number of | 0.91 (0.87-0.95) | <0.001 | 0.91 | <0.001 |
| cumulative | | | (0.87-0.94) | |
| infusions | | | | |
| Dosing group | 0.06 (0.01-0.22) | <0.001 | 0.12 | <0.001 |
| (EID, SID) | | | (0.05-0.29) | |

Abbreviations:
CI = confidence interval;
EID = extended interval dosing;
HR = hazard ratio;
IS = immunosuppressant;
PML = progressive multifocal leukoencephalopathy;
SID = standard interval dosing.
Refer to FIG. 14 and description for definitions of EID and SID in the primary, secondary, and tertiary analyses.
Statistically significant results are shown in bold.
^{a}Model includes age, sex, prior use of IS, EID/SID group, and calendar year at the start of natalizumab treatment as covariates.
Modelling could not be performed in the tertiary analysis because no PML events occurred in the tertiary analysis EID group.

Prior immunosuppressant use significantly increased PML risk. Covariate-adjusted HRs were 2.92 (95% CI 1.67-5.11; p<0.001) in the primary analysis and 2.90 (95% CI 1.60-5.27; p=0.001) in the secondary analysis (Table 32). However, the significance of this observation is limited by the small number of patients with immunosuppressant use (95 for EID-1° and 175 for EID-2°).

Sensitivity and Post Hoc Analyses

The robustness of the 3 analyses was evaluated to determine the impact of study design decisions on the results. The first sensitivity analysis examined the effect of excluding patients without known anti-JCV antibody status by including PML cases that occurred before 2012 under the assumption that all were anti-JCV antibody positive. This added 1 EID and 67 SID PML cases to the primary analysis, 5 EID and 65 SID PML cases to the secondary analysis, and 0 EID and 71 SID PML cases to the tertiary analysis. Using the same post-2012 population denominators as the planned analyses (since anti-JCV antibody status is mostly unknown for the pre-2012 population), HRs for EID vs SID ranged from <0.01 to 0.09 in all 3 analyses (Table 33).

TABLE 33
PML HR (95% CI) for EID vs SID in the sensitivity and post hoc selection bias analyses

| | | | | |
|---|---|---|---|---|
| | Sensitivity | | Post hoc analysis: | |
| | analysis: | | inclusion of PML | |
| | inclusion of | | cases without | Post hoc |
| | PML cases | Sensitivity | known anti-JCV | analysis: |
| | without known | analysis: | antibody positive | duration of |
| | anti-JCV | alternative | status and | anti-JCV |
| Planned | antibody | EID inclusion | patients with | antibody |
| analysis | positive status^{a} | criteria^{b} | dosing gaps^{c} | positive status^{d} |

Primary analysis: EID in the last 18 months of treatment
≤ 15 infusions in the last 18 months

| | | | | |
|---|---|---|---|---|
| EID-1°, n | 1989 | 998 | 7029 | 1988 |
| SID-1°, n | 13,199 | 14,122 | 17,185 | 13,132 |
| PML HR | 0.05 | 0.10 | 0.10 | 0.05 |
| (95% CI) | (0.02-0.16) | (0.02-0.45) | (0.04-0.20) | (0.01-0.18) |

Secondary analysis: EID lasting ≥ 6 months at any time in treatment history
≤ 10 infusions over 12 months

| | | | | |
|---|---|---|---|---|
| EID-2°, n | 3336 | 1870 | 9593 | 3331 |
| SID-2°, n | 15,489 | 17,902 | 16,282 | 15,424 |
| PML HR | 0.09 | 0.01 | 0.16 | 0.11 |
| (95% CI) | (0.04-0.18) | (<0.01-0.09) | (0.10-0.24) | (0.04-0.26) |

Tertiary analysis: majority of treatment received as EID
≤ 10 infusions/year over the duration of infusion history

| | | | | |
|---|---|---|---|---|
| EID-3°, n | 815 | NA | 6307 | NA |
| SID-3°, n | 23,239 | NA | 27,336 | NA |
| PML HR | <0.01^{e} | NA | 0.08 | NA |
| (95% CI) | | | (0.03-0.17) | |

Abbreviations:
CI = confidence interval;
EID = extended interval dosing;
HR = hazard ratio;
JCV = JC virus;
NA = not analyzed;
PML = progressive multifocal leukoencephalopathy;
SID = standard interval dosing.
Refer to FIG. 14 and description for definitions of EID and SID in the primary, secondary, and tertiary analyses.
^{a}PML cases assumed to be anti-JCV antibody positive and occurring before 2012 were added to the analysis populations. This added 1 EID and 67 SID cases in the primary analysis, 5 EID and 65 SID cases in the secondary analysis, and 0 EID and 71 SID cases in the tertiary analysis.
^{b}Alternative EID definitions were ≤ 13 infusions in the last 18 months in the primary analysis and ≤ 9 infusions over 12 months in the secondary analysis. An alternative definition in the tertiary analysis was not explored.
^{c}Patients with dosing gaps > 12 weeks between infusions were added to the pre-2012 PML case sensitivity analysis cohorts.

The second sensitivity analysis investigated the effect of the number of EID doses required for inclusion in EID groups by employing alternative eligibility criteria. The risk of PML was significantly lower for EID than for SID using the alternative EID inclusion criteria of ≤13 infusions in the previous 18 months (HR 0.10; 95% CI 0.02-0.45) in the primary analysis, or ≤9 infusions over 12 months (HR 0.01; 95% CI <0.01-0.09) in the secondary analysis (Table 33). Alternative EID inclusion criteria in the tertiary analysis were not explored because no EID-3° PML cases were observed.

Two post hoc analyses were carried out to address the impact of potential selection biases on the composition of the EID analysis cohorts. When the effect of excluding patients with dosing gaps (intervals >12 weeks between 2 infusions) was assessed by including patients with dosing gaps in the 3 planned analyses of PML risk, the resulting HRs ranged from 0.08 to 0.16 (Table 33).

Although all patients included in this study had tested positive for anti-JCV antibodies at least once, a second post hoc analysis was conducted to evaluate whether the duration of anti-JCV antibody seropositivity affected risk estimates. Longitudinal anti-JCV antibody status (i.e., antibody status conversion from negative to positive at some point in time) as a time-varying covariate was incorporated in the Cox regression model. The resulting HR (95% CI) estimates were 0.05 (0.11-0.18) in the primary analysis and 0.11 (0.04-0.26) in the secondary analysis (Table 33). This sensitivity analysis was not performed for the tertiary analysis.

EID was associated with a reduction in the conditional risk of PML in each successive epoch of natalizumab treatment for all 3 definitions of EID and SID (Table 34). Over the first 4 treatment epochs (≤48 infusions), only 1 PML case (in the secondary analysis) was observed in EID groups; no cases were observed in the primary and tertiary analyses. In the fifth and sixth epochs (49-72 infusions), PML risk was substantially lower for EID than for SID across all 3 analyses (Table 34).

TABLE 34
Life-table estimates of PML risk in patients included
in the primary, secondary, and tertiary analyses

| | | Estimated risk of PML per 1000 patients (no. of cases per | | | | | |
|---|---|---|---|---|---|---|---|
| | | adjusted no. of patients) | | | | | |
| Tysabri | | Primary analysis | | Secondary analysis | | Tertiary analysis | |
| exposure | # of | EID-1° | SID-1° | EID-2° | SID-2° | EID-3° | SID-3° |
| epoch^{a} | infusions | group | group | group | group | group | group |
| 1 | 1-12 | 0 | 0 | 0 | 0 | 0 | 0 |
| | | (0/1806) | (0/11,890) | (0/2980) | (0/13,049) | (0/662) | (0/18,364) |
| 2 | 13-24 | 0 | 0.28 | 0 | 0.60 | 0 | 0.52 |
| | | (0/1659) | (3/10,907) | (0/2722) | (6/9921) | (0/510) | (7/13,425) |
| 3 | 25-36 | 0 | 0.46 | 0.44 | 0.46 | 0 | 0.42 |
| | | (0/1366) | (4/8608) | (1/2292) | (3/6514) | (0/371) | (4/9603) |
| 4 | 37-48 | 0 | 2.02 | 0 | 2.58 | 0 | 1.79 |
| | | (0/1080) | (13/6439) | (0/1841) | (12/4650) | (0/265) | (13/7254) |
| 5 | 49-60 | 1.23 | 3.96 | 1.45 | 4.14 | 0 | 3.67 |
| | | (1/810) | (19/4801) | (2/1380) | (14/3385) | (0/169) | (20/5443) |
| 6 | 61-72 | 1.70 | 4.46 | 2.04 | 4.74 | 0 | 4.16 |
| | | (1/589) | (15/3363) | (2/980) | (11/2323) | (0/104) | (16/3848) |

Abbreviations:
EID = extended interval dosing;
IS = immunosuppressant;
JCV = JC virus;
PML = progressive multifocal leukoencephalopathy;
SID = standard interval dosing.
PML risk is shown as the incidence rate per 1000 patients (number of PML cases per adjusted number of patients at risk) in anti-JCV antibody positive patients without prior IS use for the primary and secondary definitions. Patients with prior IS use could not be analyzed due to an insufficient number of patients. The adjusted number of patients at risk was 95 in the EID-1° group, 689 in the SID-1° group, 171 in the EID-2° group, and 747 in the SID-2° group.
PML risk could not be calculated in the tertiary analysis of EID since no PML cases occurred in this analysis.
Refer to FIG. 14 and description for definitions of EID and SID under the primary, secondary, and tertiary analyses.
^{a}Data beyond 6 years are not shown.

EID PML Cases

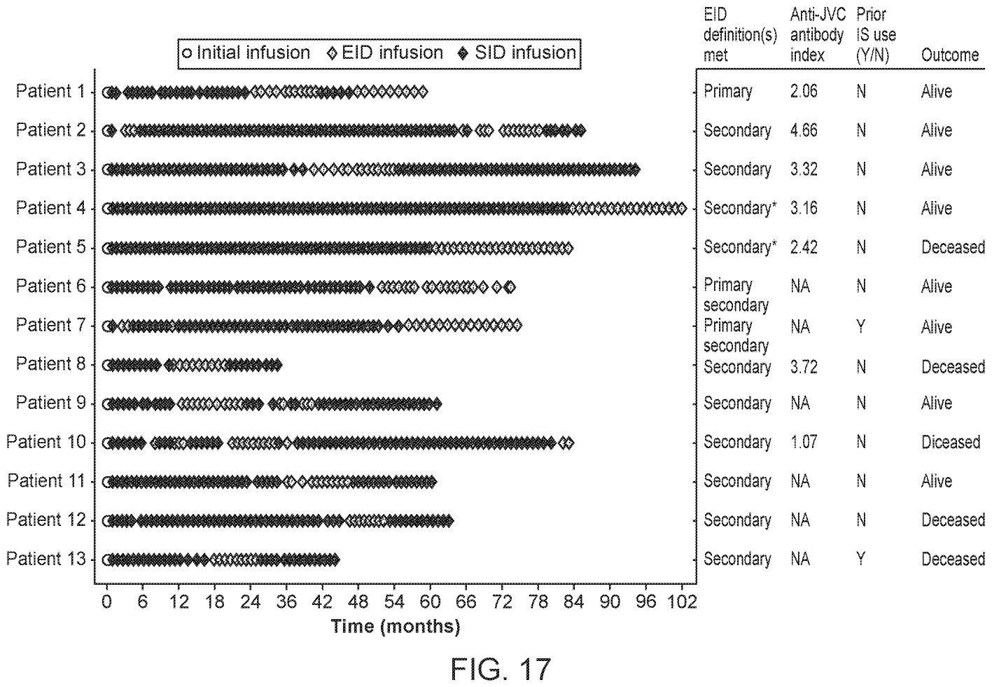

Thirteen PML cases were identified among patients meeting primary and secondary EID inclusion criteria. One case met the primary analysis criteria only, 10 cases met the secondary analysis criteria only, and 2 cases met criteria for both analyses. There were no PML cases in the tertiary analysis. At the time of PML diagnosis, 8 of 13 patients, all of whom were included in the secondary analysis, had switched back to SID from EID and had been on SID for ≥28 weeks immediately before PML diagnosis (FIG. 17). PML patients with a history of EID had longer natalizumab treatment durations, more natalizumab infusions before starting an EID regimen, and more total natalizumab infusions on average than their respective overall EID cohorts (Table 35). Prior immunosuppressant use was also more common in EID PML cases than in the overall EID cohorts (primary analysis: 33% vs 5%; secondary analysis: 17% vs 5%). Of the 7 PML cases for whom pre-PML anti-JCV antibody index values were available, 6 had index values >1.5 (FIG. 17).

TABLE 35
Characteristics of PML patients in EID and SID groups
by primary and secondary PML risk analysis groups

| | Primary analysis | | | | Secondary analysis | | | |
|---|---|---|---|---|---|---|---|---|
| | EID-1° | | SID-1° | | EID-2° | | SID-2° | |
| | | PML | | PML | | PML | | PML |
| | All | cases | All | cases | All | cases | All | cases |
| Patient Data | (1988) | (3) | (13,132) | (90) | (3331) | (12) | (15,424) | (72) |
| Female, % | 69 | 100 | 67 | 66 | 69 | 67 | 66 | 65 |
| Age at first | 42.9 | 32.3 | 44.0 | 44.5 | 43.0 | 43.1 | 43.9 | 43.4 |
| infusion, mean | (11.3) | (4.0) | (11.0) | (10.3) | (11.2) | (11.0) | (11.4) | (10.3) |
| (SD), y | | | | | | | | |
| Prior IS use, % | 5 | 33 | 5 | 16 | 5 | 17 | 5 | 15 |
| Time between | 35.5 | 33.7 | 29.7 | 29.7 | 33.5 | 31.6 | 29.4 | 29.3 |
| infusions, | (33.3, | (33.3, | (28.8, | (28.7, | (31.7, | (30.9, | (28.7, | (28.6, |
| median (Q1, | 38.8) | 35.6) | 30.8) | 30.6) | 36.9) | 32.4) | 30.5) | 30.2) |
| Q3), d | | | | | | | | |
| Total number | 50 | 68 | 46 | 60 | 51 | 68 | 27 | 58 |
| of infusions, | (31, | (50, | (28, | (47, | (31, | (58, | (17, | (42, |
| median (Q1, | 75.5) | 68) | 70) | 73) | 75) | 83) | 53) | 70) |
| Q3) | | | | | | | | |
| Total duration | 59 | 74 | 44 | 58.5 | 56 | 74.5 | 26 | 54 |
| of natalizumab | (37, | (58, | (27, | (47, | (36, | (59.5, | (16, | (40.5, |
| treatment, | 87) | 75) | 68) | 71) | 81) | 85) | 51) | 66) |
| median (Q1, | | | | | | | | |
| Q3), mo | | | | | | | | |
| Number of | 37 | 54 | 27 | 41 | 25 | 40.5 | 1 | 1 |
| natalizumab | (18, | (37, | (9, | (29, | (13, | (19, | (1, 1) | (1, 1) |
| infusions | 63) | 55) | 51) | 54) | 44) | 56.5) | | |
| before EID/SID | | | | | | | | |
| regimen start, | | | | | | | | |
| median (Q1, | | | | | | | | |
| Q3) | | | | | | | | |

Abbreviations: EID = extended interval dosing; IS = immunosuppressant; Q1 = first quartile; Q3 = third quartile; SID = standard interval dosing.
Refer to FIG. 14 and description for definitions of EID and SID in the primary, secondary, and tertiary analyses.
Characteristics of patients in the tertiary analysis are not shown, as no PML cases occurred in this analysis.

Discussion

To address the question of PML risk with EID, we conducted a retrospective cohort study using patient data collected by the TOUCH program. This is the largest study of PML risk associated with natalizumab EID to date and also provides an example of how real-world data derived from a REMS program can be rigorously analyzed to address a clinically meaningful question of risk reduction. Even though PML is an uncommon event, the size of the TOUCH dataset provided sufficient power to produce robust and statistically significant results. We evaluated PML risk in anti-JCV antibody positive patients who met any of the 3 inclusion criteria for natalizumab EID vs the risk in patients on SID using 3 different pre-specified analyses to investigate a wide range of dosing patterns utilized in real-world clinical practice. For each of the 3 different analyses, there was a substantial reduction in PML risk with natalizumab EID compared with SID.

The difference in dosing intervals between the overall EID and SID groups was relatively modest (ADI of 35-43 days for EID vs 30-31 days for SID). While these values combine different treatment practices and dosing patterns, the results suggest that extending dosing intervals by as little as 1-2 weeks may produce a large reduction in PML risk. It is unlikely that the main conclusions of this study were affected by outliers at either end of the ADI range, as patients with any dosing interval <3 weeks or >12 weeks in their history were excluded from the planned analyses and third-quartile (75th-percentile) ADI ranges for the EID cohort were 37-45 days.

Prespecified sensitivity analyses were performed to evaluate the impact of the following 2 study design elements on the results: (1) the inclusion of only patients with known positive anti-JCV antibody status and (2) the number of EID infusions required for inclusion in the EID-1° or EID-2° groups. In addition, a post hoc sensitivity analysis was performed to evaluate the impact of excluding patients with a history of dosing gaps (>12 weeks between doses). The results of both sensitivity and post hoc analyses were comparable to those of the 3 planned analyses, demonstrating the robustness of the risk estimates and further strengthening the main conclusion that natalizumab EID is associated with lower PML risk than SID in at-risk patients.

The possibility that physicians are more likely to switch patients with longer durations of JCV seropositivity to EID created a potential selection bias in the composition of the EID cohorts. When anti-JCV antibody positivity status was accounted for as a time-varying covariate in a second post hoc analysis, the resulting HRs and 95% CIs were similar to those produced in the original prespecified analyses, indicating that this potential bias did not impact the main study conclusions.

The identification of 13 PML cases among the combined 5249 patients in the EID-1° and/or EID-2° groups indicates that while these EID regimens are associated with significantly lower PML risk than SID, the risk is not completely eliminated. No cases of PML were observed with the more stringent tertiary analysis, though the EID-3° group was relatively small (n=815). Most of the EID PML cases described here had multiple risk factors for PML, including longer overall natalizumab treatment duration, longer periods of SID before switching to EID, and a greater likelihood of prior immunosuppressant use than patients in the corresponding overall EID cohorts. In addition, several of the EID PML cases had returned to SID prior to PML diagnosis. In a previously published case report of PML in a patient receiving natalizumab EID, the affected patient also had elevated PML risk factors, including a prolonged period of SID preceding EID and an anti-JCV antibody index >1.5.¹³

The biological mechanisms underlying the observed PML risk reduction require additional research, but partial reversal of natalizumab pharmacodynamic effects, including decreased receptor saturation, increased soluble vascular cell adhesion molecule expression, and a reduction in natalizumab-induced peripheral lymphocytosis, have been reported to occur 4-8 weeks after the last dose¹⁴ and may allow for the reestablishment of some immune surveillance in the central nervous system.

The conclusions of this study are limited by several inherent biases. EID patients received more doses of natalizumab than SID patients, which could have introduced a selection bias favoring more PML cases in the EID cohorts, since natalizumab exposure is a known PML risk factor. Conversely, most patients had received >2 years of SID treatment without developing PML prior to starting EID, so the EID groups may have included patients with inherently reduced risk of PML, thereby introducing a selection bias in favor of fewer PML cases in the EID groups. Also, anti-JCV antibody index data are not available for all patients in TOUCH; therefore, we do not know whether index values differ between the EID and SID cohorts and whether any such difference plays a role in the risk reductions observed in this study. Because EID is used as an off-label strategy in clinical practice to reduce PML risk, anti-JCV antibody index values might be higher in EID than SID patients, as was observed in a retrospective study of natalizumab EID.¹¹ If this is true, the risk reductions seen in the EID cohorts described here could potentially be even larger in EID and SID patient populations with the same distributions of anti-JCV antibody index values.

Finally and most importantly, as the TOUCH program does not collect information about therapeutic efficacy, we could not assess the benefit-risk profile of EID compared with SID. Several studies of patient outcomes following natalizumab discontinuation indicate that MS disease activity is suppressed for at least 6 weeks and possibly as long as 12 weeks after the last administration.¹⁵⁻¹⁸ Furthermore, 2 retrospective studies have suggested that natalizumab efficacy is not compromised by EID regimens.^{10, 11} However, the findings from the latter studies are limited by non-randomized designs, small study populations, variable dosing practices, and potential selection biases in the EID study populations. In contrast to the clinical results, model-based simulations of natalizumab exposure have suggested that EID regimens (with 6-8-week intervals) may not confer adequate protection from MS disease activity.¹⁹ In light of uncertainties about the long-term benefit-risk profile of natalizumab EID, it is premature to suggest that SID should be replaced by EID. A planned randomized prospective study of EID vs SID will yield a more comprehensive understanding of both the effectiveness and the safety of natalizumab EID.

### REFERENCES

1. Miller D H, Khan O A, Sheremata W A, et al. A controlled trial of natalizumab for relapsing multiple sclerosis. N Engl J Med 2003;348:15-23.

2. Polman C H, O'Connor P W, Havrdova E, et al. A randomized, placebo-controlled trial of natalizumab for relapsing multiple sclerosis. N Engl J Med 2006; 354:899-910.

3. Prosperini L, Sacca F, Cordioli C, et al. Real-world effectiveness of natalizumab and fingolimod compared with self-injectable drugs in non-responders and in treatment-naive patients with multiple sclerosis. J Neurol 2017;264:284-294.

4. Butzkueven H, Kappos L, Pellegrini F, et al. Efficacy and safety of natalizumab in multiple sclerosis: interim observational programme results. J Neurol Neurosurg Psychiatry 2014; 85:1190-1197.

5. Rudick R A, Sandrock A. Natalizumab: a4-integrin antagonist selective adhesion molecule inhibitors for MS. Expert Rev Neurother 2004; 4:571-580.

6. Stuve O, Bennett J L. Pharmacological properties, toxicology and scientific rationale for the use of natalizumab (Tysabri) in inflammatory diseases. CNS Drug Rev 2007; 13:79-95.

7. Tysabri® (natalizumab) [prescribing information]. Cambridge, Mass.: Biogen, 2018.

8. Bloomgren G, Richman S, Hotermans C, et al. Risk of natalizumab-associated progressive multifocal leukoencephalopathy. N Engl J Med 2012; 366:1870-1880.

9. Ho P-R, Koendgen H, Campbell N, Haddock B, Richman S, Chang I. Risk of natalizumab-associated progressive multifocal leukoencephalopathy in patients with multiple sclerosis: a retrospective analysis of data from four clinical studies. Lancet Neurol 2017; 16:925-933.

10. Bomprezzi R, Pawate S. Extended interval dosing of natalizumab: a two-center, 7-year experience. Ther Adv Neurol Disord 2014; 7:227-231.

11. Zhovtis Ryerson L, Frohman T C, Foley J, et al. Extended interval dosing of natalizumab in multiple sclerosis. J Neurol Neurosurg Psychiatry 2016; 87:885-889.

12. Risk Evaluation and Mitigation Strategy (REMS): TYSABRI Outreach: Unified Commitment to Health (TOUCH®) Prescribing Program [online]. Available at: www.fda.gov/downloads/Drugs/DrugSafety/PostmarketDrugSafetyInformationforPatientsand Providers/UCM288126.pdf. Accessed May 19, 2018.

13. Hervás-Garcia J V, Presas-Rodriguez S, Crespo-Cuevas A M, et al. Progressive multifocal leukoencephalopathy associated to natalizumab extended dosing regimen. Neurodegenr Dis Manag 2015; 5:399-402.

14. Plavina T, Muralidharan K K, Kuesters G, et al. Reversibility of the effects of natalizumab on peripheral immune cell dynamics in MS patients. Neurology 2017; 89:1584-1593.

15. Fox R J, Cree B A, De Seze J, et al. MS disease activity in RESTORE: a randomized 24-week natalizumab treatment interruption study. Neurology 2014; 82:1491-1498.

16. Kappos L, Radue E W, Comi G, et al. Switching from natalizumab to fingolimod: A randomized, placebo-controlled study in RRMS. Neurology 2015; 85:29-39.

17. Grimaldi L M, Prosperini L, Vitello G, Borriello G, Fubelli F, Pozzilli C. MRI-based analysis of the natalizumab therapeutic window in multiple sclerosis. Mult Scler 2012; 18:1337-1339.

18. Berkovich R, Togasaki D M, Cen S Y, Steinman L. CD4 cell response to interval therapy with natalizumab. Ann Clin Transl Neurol 2015; 2:570-574.

19. Muralidharan K K, Steiner D, Amarante D, et al. Exposure-disease response analysis of natalizumab in subjects with multiple sclerosis. J Pharmacokinet Pharmacodyn 2017; 44:263-275.

### Example 8: Evaluating the Efficacy and Safety of 6-Week Extended Interval Dosing of Natalizumab via a Prospective, Controlled, Randomized, Open-label, Rater-blinded Phase 3b Study (NOVA)

Introduction

Natalizumab, a highly efficacious therapy for relapsing-remitting multiple sclerosis (RRMS), is also associated with risk of PML. (1-5) A recent analysis of the TOUCH dataset demonstrated that EID is associated with significantly lower PML risk than standard interval dosing (SID) in anti-JC virus antibody positive patients. (6) To date, there have been no randomized studies to compare the efficacy of natalizumab EID and SID. In the absence of prospective, randomized efficacy data, no benefit-risk profile has been established for EID.

Objective

To describe the design of a phase 3b study to evaluate the efficacy of switching to EID natalizumab after a stable period of SID compared with continuing SID.

Methods

Natalizumab, phase 3b, prospective, randomized, Open-label study comparing extended interval dosing Versus Approved dose (NOVA) will be an interventional, controlled, rater-blinded global study (clinicaltrials.gov no. NCT03689972). Patient inclusion criteria include age 18-60 years, an Expanded Disability Status Scale score ≤5.5, a diagnosis of RRMS, stability on natalizumab SID (having received ≥11 doses and having had no relapses in the prior 12 months), no prior immunosuppressant use, and no gadolinium-enhancing (Gd+) lesions at screening.

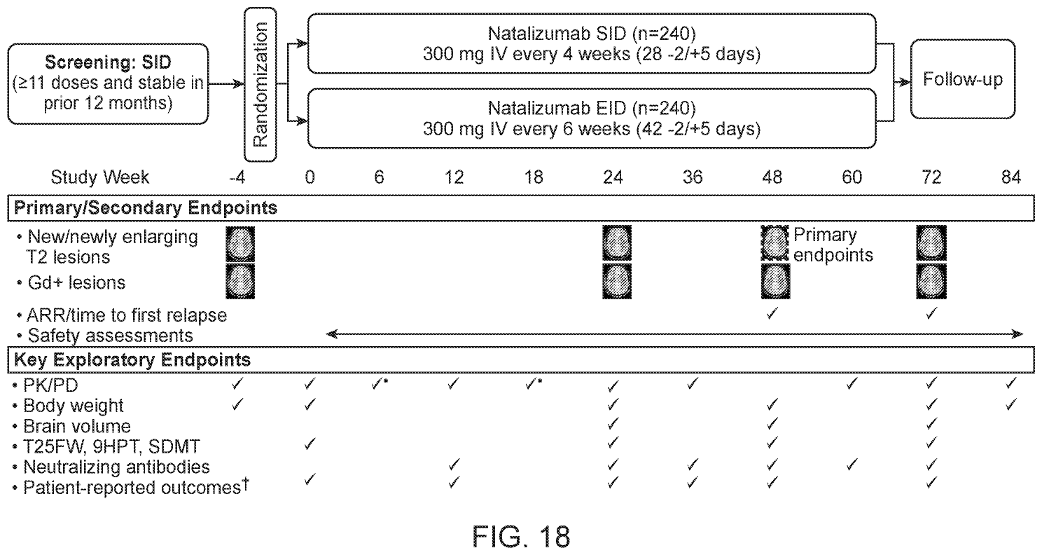

Approximately 480 patients will be enrolled in NOVA. Patients will be randomized 1:1 to natalizumab SID (300 mg intravenous [IV] every 4 weeks [26-33 days]) or EID (300 mg IV every 6 weeks [40-47 days]). Study duration will be 88 weeks (4 weeks screening, 72 weeks randomized treatment, and 12 weeks follow-up) (FIG. 18).

The primary endpoint is the number of new/newly enlarging T2 lesions at 48 weeks. Key secondary endpoints include time to relapse, relapse rate, the number of new radiologic lesions, and the incidence of serious adverse events. Exploratory endpoints include Timed 25-Foot Walk (T25FW), 9-Hole Peg Test (9HPT), and Symbol Digit Modality Test (SDMT) scores and confirmed disability worsening or improvement.

Data on natalizumab serum concentration, alpha-4 integrin saturation, lymphocyte counts, and body weight will be collected to explore relationships between pharmacokinetics (PK)/pharmacodynamics (PD) and efficacy.

Study Rationale

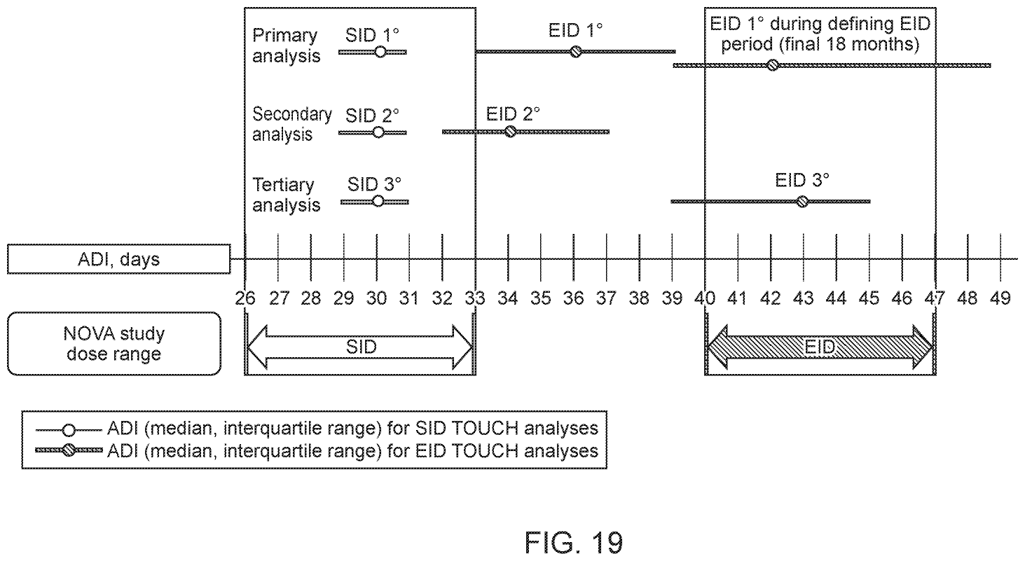

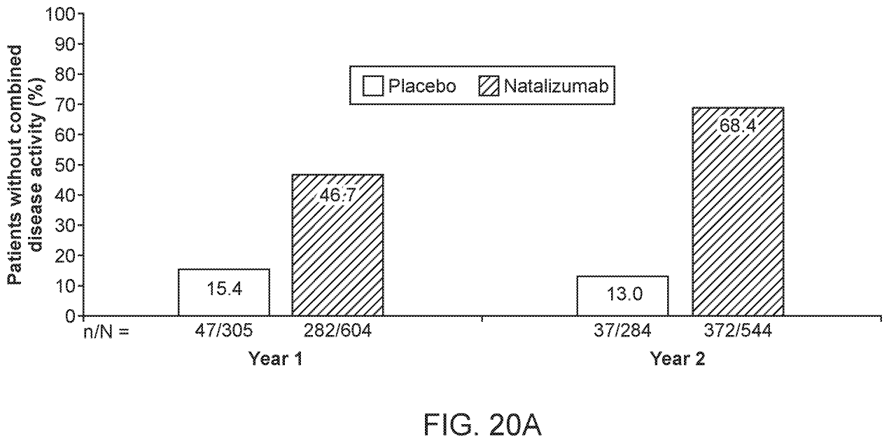

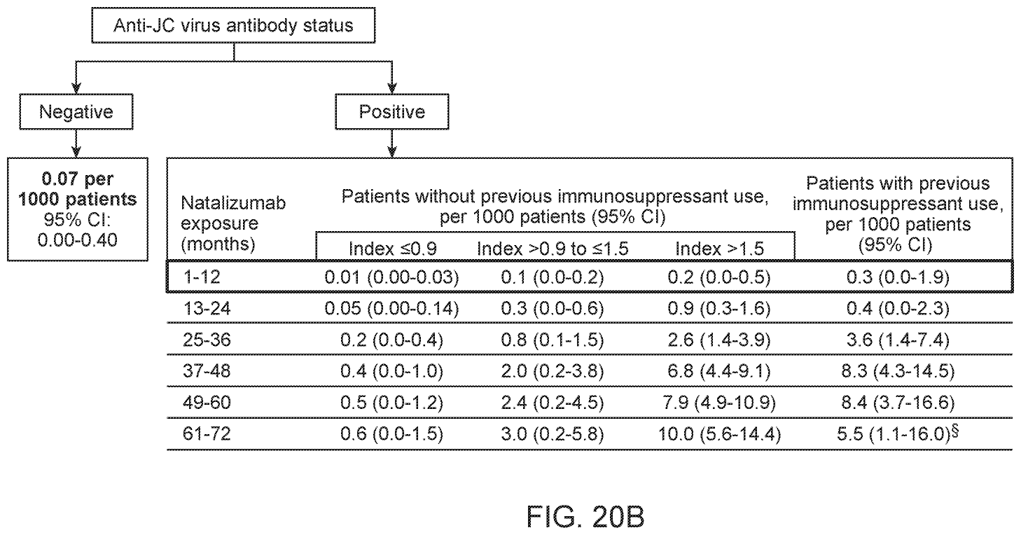

The EID intervals in NOVA were chosen to encompass the real-world dosing intervals associated with the lower risk of PML observed in the TOUCH analysis (FIG. 19). (6) The rationale for the requirement of ≥12 months of disease stability on SID prior to random allocation and switching to EID is as follows: Independent studies suggest comparable efficacy between SID and EID in patients switching to EID after 1-2 years of SID. (7-9) Modeling shows that initiating patients on EID may result in inadequate protection from clinical and magnetic resonance imaging (MRI) disease activity. (10) Analysis of patients in AFFIRM demonstrates that the efficacy of natalizumab improves after the first year of treatment (FIG. 20A). (11)

Analysis of a pooled cohort of patients from four open-label studies of natalizumab indicates that the risk of PML in the first year of treatment is low regardless of index or prior use of immunosuppressants (FIG. 20B). (12) Thus, the present inventors hypothesize that the incentive for EID as a PML risk mitigation strategy is low during the first year of treatment.

The number of new or newly enlarging T2 hyperintense lesions at 48 weeks for the primary endpoint selection is an objective and sensitive measure of natalizumab efficacy. In open-label trials, rater-blinded MRI endpoints remain fully objective, while relapse-based endpoints are more prone to bias in these contexts. T2 hyperintense lesions represent a persistent footprint of demyelination and provide high-sensitivity detection of disease activity. (13) The sample size (N=480) provides >80% power to detect a difference between 0.3 (the predicted value for SID group in this population) and 0.5 in mean new or newly enlarging T2 lesions.

Literature

EID is practiced by some physicians as an off-label strategy to mitigate the risk of natalizumab-associated PML. Several retrospective studies have suggested that natalizumab efficacy may be maintained with EID dosing schedules >4 weeks. (7,8) However, partial reversal of natalizumab's pharmacodynamic effects has been reported to occur 4-8 weeks after the last dose. (17) This study will provide the first randomized, controlled efficacy data for patients treated with natalizumab EID and will yield a more comprehensive understanding of both the effectiveness and the safety of natalizumab EID.

### REFERENCES

1. Miller D H, et al. N Engl J Med. 2003; 348:15-23;

2. Polman C H, et al. N Engl J Med. 2006; 354:899-910;

3. Prosperini L, et al. J Neurol. 2017; 264:284-294;

4. Butzkueven H, et al. J Neurol Neurosurg Psychiatry. 2014; 85:1190-1197;

5. TYSABRI® (natalizumab) [prescribing information]. Cambridge, Mass.: Biogen; 2018;

6. Zhovtis Ryerson L, et al. Presented at ACTRIMS; Feb. 1-3, 2018; San Diego, Calif. LB250;

7. Zhovtis Ryerson L, et al. J Neurol Neurosurg Psychiatry. 2016; 87:885-889;

8. Bomprezzi R, Pawate S, Ther Adv Neurol Disord. 2014; 7:227-231;

9. Muralidharan K K, et al. Presented at ECTRIMS; Sep. 14-17, 2016; London, UK. P1672;

10. Muralidharan K K, et al. J Pharmacokinet Pharmacodyn. 2017; 44:263-275;

11. Havrdová E, et al. Lancet Neurol. 2009; 8:254-260;

12. Ho P R, et al. Lancet Neurol. 2017; 16:925-933;

13. Rovira A, et al. Ther Adv Neurol Disord. 2013; 6:298-310;

14. Campagnolo D, et al. Neurology. 2016; 87(2):e25;

15. O'Connor P, et al. Neurology. 2014; 83:78-86;

16. Foley J, et al. Mult Scler. 2016; 22(Suppl 3):646-647. P1229;

17. Plavina T et al. Neurology. 2017; 89:1584-1593.

All references, patents and patent applications disclosed herein are incorporated by reference in the entirety and for all purposes, and in particularly with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean ±10% of the recited numerical value.

Where a range of values is provided, each value between the upper and lower ends of the range are specifically contemplated and described herein.

### Embodiments

Exemplary embodiments include a method of reducing risk of developing progressive multifocal leukemia (PML) in a subject, comprising: a. identifying a low PML risk subject who has been receiving natalizumab therapy on a standard interval dosing (SID) schedule of 4-week intervals; b. determining whether the subject has switched from a low PML risk subject to a high PML risk subject during the natalizumab therapy; and c. if the subject has switched to a high PML risk subject, identifying the high PML risk subject for natalizumab therapy on an extended interval dosing (EID) schedule of greater than 4-week intervals (e.g., at least 5-week intervals).

Exemplary embodiments include a method comprising administering natalizumab to a subject having an anti-JCV antibody index level of less than or equal to 0.9 with a SID schedule of 4-week intervals, and then after the subject has an anti-JCV antibody index level of greater than 1.5, administering natalizumab therapy on an extended interval dosing (EID) schedule of greater than 4-week intervals (e.g., at least 5-week intervals) to the subject.

Exemplary embodiments include a method of reducing risk of developing PML in a subject, comprising: a. administering to a subject a therapeutically effective amount of natalizumab on a SID schedule of 4-week intervals, wherein the subject is a low PML risk subject; b. determining whether the subject has switched from a low PML risk subject to a high PML risk subject during the SID natalizumab therapy; and c. if the subject has switched to a high PML risk subject, administering to the subject a therapeutically effective amount of natalizumab on an EID schedule of at least 5-week intervals.

Exemplary embodiments include a method of reducing risk of developing PML in a subject, comprising identifying a subject for natalizumab therapy on an EID schedule of at least 5-week intervals, wherein the subject has tested seropositive for anti-JCV antibodies and has received natalizumab therapy on a SID schedule of 4-week intervals.

Exemplary embodiments include a method of reducing risk of developing PML in a subject, comprising administering to a subject a therapeutically effective amount of natalizumab on an EID schedule of at least 5-week intervals, wherein the subject has tested seropositive for anti-JCV antibodies and has been receiving natalizumab therapy on a SID schedule of 4-week intervals.

Exemplary embodiments include a method of reducing risk of developing PML in a subject, comprising: a. identifying a subject who has tested seropositive for anti-JCV antibodies and has been receiving natalizumab therapy on a SID schedule of 4-week intervals; and b. administering to the subject a therapeutically effective amount of natalizumab on an EID schedule of at least 5-week intervals.

Exemplary embodiments include a method of treating multiple sclerosis (MS) in a subject, comprising: a. identifying a low PML risk subject having MS who has been receiving natalizumab therapy on a standard interval dosing (SID) schedule of 4-week intervals; b. determining whether the subject has switched from a low PML risk subject to a high PML risk subject during the natalizumab therapy; and c. if the subject has switched to a high PML risk subject, identifying the high PML risk subject for natalizumab therapy on an extended interval dosing (EID) schedule of at least 5-week intervals.

Exemplary embodiments include a method of treating Crohn's disease in a subject, comprising: a. identifying a low PML risk subject having Crohn's disease who has been receiving natalizumab therapy on a standard interval dosing (SID) schedule of 4-week intervals; b. determining whether the subject has switched from a low PML risk subject to a high PML risk subject during the natalizumab therapy; and c. if the subject has switched to a high PML risk subject, identifying the high PML risk subject for natalizumab therapy on an extended interval dosing (EID) schedule of at least 5-week intervals.

Exemplary embodiments include a method of treating epilepsy in a subject, comprising: a. identifying a low PML risk subject having epilepsy who has been receiving natalizumab therapy on a standard interval dosing (SID) schedule of 4-week intervals; b. determining whether the subject has switched from a low PML risk subject to a high PML risk subject during the natalizumab therapy; and c. if the subject has switched to a high PML risk subject, identifying the high PML risk subject for natalizumab therapy on an extended interval dosing (EID) schedule of at least 5-week intervals.

In some cases of one or more exemplary embodiments, step (a) comprises identifying a low PML risk subject who has an anti-JCV antibody index level of less than or equal to 0.9. In some cases of one or more exemplary embodiments, step (b) comprises determining the anti-JCV antibody index level of the subject. In some cases of one or more exemplary embodiments, the high PML risk subject of step (c) has an anti-JCV antibody index level of greater than 1.5. In some cases of one or more exemplary embodiments, the high PML risk subject of step (c) has an anti-JCV antibody index level of greater than 0.9.

In some cases of one or more exemplary embodiments, the subject being administered natalizumab therapy on an extended interval dosing (EID) schedule has an anti-JCV antibody index level of greater than 1.5. In some cases of one or more exemplary embodiments, the subject being administered natalizumab therapy on an extended interval dosing (EID) schedule has an anti-JCV antibody index level of greater than 0.9. In some cases of one or more exemplary embodiments, the subject being administered natalizumab therapy on a standard interval dosing (SID) schedule has an anti-JCV antibody index level of less than or equal to 0.9.

In some cases of one or more exemplary embodiments, sample(s) from the subject being administered natalizumab therapy on an extended interval dosing (EID) schedule previously obtained from the subject, e.g., after having been administered natalizumab therapy on standard interval dosing (SID), have an anti-JCV antibody index level of greater than 1.5. In some cases of one or more exemplary embodiments, sample(s) from the subject being administered natalizumab therapy on an extended interval dosing (EID) schedule previously obtained from the subject, e.g., after having been administered natalizumab therapy on standard interval dosing (SID), have an anti-JCV antibody index level of greater than 0.9. In some cases of one or more exemplary embodiments, sample(s) from the subject being administered natalizumab therapy on an extended interval dosing (EID) schedule previously obtained from the subject, e.g., before extended interval dosing (EID), have an anti-JCV antibody index level of less than or equal to 0.9.

In some cases of one or more exemplary embodiments, the natalizumab therapy on an EID schedule is at 5- to 10-week intervals. In some cases of one or more exemplary embodiments, the natalizumab therapy on an EID schedule is at 5- to 8-week intervals. In some cases of one or more exemplary embodiments, the natalizumab therapy on an EID schedule is at an interval of from greater than 4 weeks to no more than 12 weeks. In some cases of one or more exemplary embodiments, the natalizumab therapy on an EID schedule is at an interval of from greater than 4 weeks to no more than 10 weeks. In some cases of one or more exemplary embodiments, the natalizumab therapy on an EID schedule is at an interval of from greater than 4 weeks to no more than 8 weeks. In some cases of one or more exemplary embodiments, the natalizumab therapy on an EID schedule is at an interval of from greater than 4 weeks to no more than 6 weeks. In some cases of one or more exemplary embodiments, the natalizumab therapy on an EID schedule is at 6-week intervals. In some cases of one or more exemplary embodiments, the natalizumab therapy on an EID schedule is at 7-week intervals. In some cases of one or more exemplary embodiments, the natalizumab therapy on an EID schedule is at 8-week intervals. In some cases of one or more exemplary embodiments, the natalizumab therapy on an EID schedule is at 9-week intervals.

In some cases of one or more exemplary embodiments, the subject has been receiving natalizumab therapy on a SID schedule at 4-week intervals for at least six months, one year, 18 months, 2 years, or 5 years. In some cases of one or more exemplary embodiments, the subject receives natalizumab therapy on a SID schedule at 4-week intervals for at least at least six months, one year, 18 months, 2 years, or 5 years. In some cases of one or more exemplary embodiments, the subject is administered natalizumab therapy on a SID schedule at 4-week intervals for at least at least six months, one year, 18 months, 2 years, or 5 years.

In some cases of one or more exemplary embodiments, the subject has been diagnosed with an autoimmune condition. In some cases of one or more exemplary embodiments, the subject has an autoimmune condition. In some cases of one or more exemplary embodiments, the subject is treated for an autoimmune condition with the natalizumab therapy. In some cases of one or more exemplary embodiments, the autoimmune condition is multiple sclerosis. In some cases of one or more exemplary embodiments, the autoimmune condition is Crohn's disease. In some cases of one or more exemplary embodiments, the autoimmune condition is rheumatoid arthritis.

In some cases of one or more exemplary embodiments, the subject has been diagnosed with epilepsy. In some cases of one or more exemplary embodiments, the subject has epilepsy. In some cases of one or more exemplary embodiments, the subject is treated for an autoimmune condition with the natalizumab therapy.

In some cases of one or more exemplary embodiments, the subject has a prior history of immunosuppression. In some cases of one or more exemplary embodiments, the subject was treated with an immunosuppressant prior to receiving natalizumab therapy on a SID schedule of 4-week intervals. In some cases of one or more exemplary embodiments, a single dose of natalizumab is 300 mg. In some cases of one or more exemplary embodiments, the risk of developing PML in the subject is reduced by at least 10% relative to the risk of developing PML in a subject receiving natalizumab therapy on a SID schedule of 4-week intervals. In some cases of one or more exemplary embodiments, the risk of developing PML in the subject is reduced by at least 20% relative to the risk of developing PML in a subject receiving natalizumab therapy on a SID schedule of 4-week intervals. In some cases of one or more exemplary embodiments, the risk of developing PML in the subject is reduced by at least 50% relative to the risk of developing PML in a subject receiving natalizumab therapy on a SID schedule of 4-week intervals.

In some cases of one or more exemplary embodiments, the efficacy of the natalizumab therapy on the EID schedule is, or is at least, 80% of the efficacy of natalizumab therapy on the SID schedule. In some cases of one or more exemplary embodiments, the efficacy of the natalizumab therapy on the EID schedule is, or is at least, 90% of the efficacy of natalizumab therapy on the SID schedule.

In some cases of one or more exemplary embodiments, the efficacy of the natalizumab therapy comprises maintenance of trough a4-integrin saturation (>50%). In some cases of one or more exemplary embodiments, the natalizumab therapy on the EID schedule maintains a trough a4-integrin saturation of at least, or of at least about, 65%. In some cases of one or more exemplary embodiments, the efficacy of the natalizumab therapy comprises a mean risk of Gd+ lesions at week 48 of natalizumab therapy on the EID and/or SID schedule. In some cases of one or more exemplary embodiments, the natalizumab therapy on the EID schedule exhibits a mean risk of Gd+ lesions at week 48 of natalizumab therapy on the EID schedule of less than about 20%, 15%, 10%, or 5%. In some cases of one or more exemplary embodiments, the natalizumab therapy on the EID schedule exhibits a mean risk of Gd+ lesions at week 48 of natalizumab therapy on the EID schedule of from about 0.1% to about 20%, from about 0.1% to about 15%, from about 0.1% to about 10%, or from about 0.1% to about 5%.

In some cases of one or more exemplary embodiments, the efficacy of the natalizumab therapy comprises a mean expected number of Gd+ lesions at week 48 of natalizumab therapy on the EID and/or SID schedule. In some cases of one or more exemplary embodiments, the natalizumab therapy on the EID schedule exhibits a mean expected number of Gd+ lesions at week 48 of natalizumab therapy on the EID schedule of less than about 1.5, 1.25, 1, 0.8, 0.65, 0.4, 0.2, or 0.15. In some cases of one or more exemplary embodiments, the natalizumab therapy on the EID schedule exhibits a mean expected number of Gd+ lesions at week 48 of natalizumab therapy on the EID schedule of from about 0 to about 1.5, from about 0 to about 1.25, from about 0 to about 1, from about 0 to about 0.8, from about 0 to about 0.65, from about 0 to about 0.4, from about 0 to about 0.2, or from about 0 to about 0.15.

In some cases of one or more exemplary embodiments, the efficacy of the natalizumab therapy comprises a cumulative probability of relapse at week 48 of natalizumab therapy on the EID and/or SID schedule. In some cases of one or more exemplary embodiments, the natalizumab therapy on the EID schedule exhibits a cumulative probability of relapse at week 48 of natalizumab therapy on the EID schedule of less than about 30%, 27%, 25%, 20%, 15%, or 10%. In some cases of one or more exemplary embodiments, the natalizumab therapy on the EID schedule exhibits a cumulative probability of relapse at week 48 of natalizumab therapy on the EID schedule of from about 5% to about 30%, from about 5% to about 25%, from about 5% to about 20%, or from about 5% to about 15%.

In some cases of one or more exemplary embodiments, the subject is less than about 120 kg in weight. In some cases of one or more exemplary embodiments, the subject is less than about 100 kg in weight. In some cases of one or more exemplary embodiments, the subject is less than about 99 kg in weight. In some cases of one or more exemplary embodiments, the subject is less than about 80 kg in weight. In some cases of one or more exemplary embodiments, the subject at least about 40 kg in weight. In some cases of one or more exemplary embodiments, the subject is from at least about 40 kg in weight to less than about 120 kg in weight. In some cases of one or more exemplary embodiments, the subject is from at least about 40 kg in weight to less than about 100 kg in weight. In some cases of one or more exemplary embodiments, the subject is from at least about 40 kg in weight to less than about 80 kg in weight.

What is claimed is:

1. A method of improving the safety of chronic natalizumab therapy in a patient in need thereof, comprising determining whether the patient has at least one risk factor for progressive multifocal encephalopathy (PML), and in the presence of said at least one risk factor administering natalizumab to the patient on an EID schedule comprising at least 5 week intervals.

2. The method according to claim 1, wherein said at least one risk factor comprises prior immunosuppression of the patient.

3. The method according to any one of the preceding claims, wherein said at least one risk factor comprises or further comprises the presence of serum anti-JCV antibodies in the patient.

4. The method according to any one of the preceding claims, wherein said at least one risk factor comprises the presence of anti-JCV antibodies in the patient, said determining step comprises determining the anti-JCV antibody status of the patient, and if the patient is seropositive for JCV antibodies then administering natalizumab to the patient on an EID schedule of at least 5 week intervals.

5. The method according to any one of the preceding claims, wherein the patient has an anti-JCV antibody index of greater than 0.9.

6. The method according to claim 5, wherein the patient has an anti-JCV antibody index level greater than 1.5.

7. The method according to any one of the preceding claims, wherein said at least one risk factor comprises the length of prior natalizumab treatment, and if the patient has undergone more than six months of natalizumab therapy then the method comprises administering natalizumab to the patient on an EID schedule of at least 5 week intervals.

8. The method according to any one of the preceding claims, wherein said at least one risk factor comprises the length of prior natalizumab treatment, the patient has undergone more than six months of natalizumab therapy, and the method comprises administering natalizumab to the patient on an EID schedule of at least 5 week intervals.

9. The method according to claim 7 or 8, wherein said more than six months of natalizumab therapy is more than six months of natalizumab therapy on a SID schedule.

10. The method according to any one of the preceding claims, wherein the interval of the EID schedule is from 5 weeks to 8 weeks.

11. The method according to any one of the preceding claims, wherein the interval of the EID schedule is from 5 to 7 weeks.

12. The method according to any one of the preceding claims, wherein the interval of the EID schedule is 6 weeks.

13. The method according to any one of the preceding claims, wherein the EID schedule maintains a mean trough a4-integrin receptor saturation of greater than 50% in an EID patient population.

14. The method of claim 13, wherein the EID schedule maintains a mean trough α4β1-integrin receptor saturation of greater than 65% in an EID patient population.

15. The method according to any one of the preceding claims, wherein
a. the patient is less than about 120 kg in weight; or
b. the patient is less than about 100 kg in weight; or
c. the patient is less than about 80 kg in weight; or
d. the patient is from about 40 kg to less than about 120 kg in weight; or
e. the patient is from about 40 kg to less than about 80 kg in weight; or
f. the patient is from about 40 kg to less than about 60 kg in weight.

16. The method according to claim 15, wherein the patient is from about 40 kg to about 80 kg in weight and the EID schedule has an interval of at least 5 weeks and no more than 7 weeks.

17. The method of claim 16, wherein the patient is from about 40 kg to about 80 kg in weight and the EID schedule has an interval of 6 weeks.

18. The method of claim 16, wherein the patient is from about 40 kg to about 60 kg in weight and the EID schedule has an interval of from 6 weeks to 7 weeks, preferably 6 weeks.

19. The method according to any one of the preceding claims, wherein the EID schedule comprises a dose of 300 milligrams.

20. The method according to any one of claims 1-18, wherein the EID schedule comprises a dose equivalent to 3.75 to 7.5 mg natalizumab/kg patient body weight.

21. The method according to any one of the preceding claims, wherein the patient has an autoimmune disease.

22. The method according to claim 21, wherein the autoimmune disease is MS.

23. The method according to claim 21, wherein the autoimmune disease is an inflammatory bowel disease.

24. The method according to claim 21, wherein the autoimmune disease is Crohn's disease.

25. The method according to any one of claims 1 to 20, wherein the patient has epilepsy.

26. A method of administering to a patient in need thereof a natalizumab therapy, the method comprising: administering the natalizumab therapy on an EID schedule, wherein
a. the patient has a weight range of from 40 kg to less than 80 kg; and
b. the PML risk of the natalizumab therapy is reduced as compared to natalizumab therapy on an SID schedule.

27. The method of claim 26, wherein the EID schedule comprises a dose of 300 milligrams.

28. The method according to claim 26 or 27,wherein the EID schedule comprises a dose equivalent to 3.75 to 7.5 mg natalizumab/kg patient body weight.

29. The method according to claim 26, 27, or 28, wherein the efficacy of the natalizumab therapy on the EID schedule is reduced by no more than 20% as compared to natalizumab therapy on an SID schedule.

30. The method according to any one of claims 26-29, wherein the risk of a Gd+ lesion at week 48 of natalizumab therapy on the EID schedule is increased by no more than about 10%, expected mean number of Gd+ lesions at week 48 of natalizumab therapy on the EID schedule is increased by no more than about 0.65, and/or the cumulative probability of a clinical relapse at week 48 of natalizumab therapy on the EID schedule is increased by no more than about 15%.

31. The method according to any one of claims 26-30, wherein the EID schedule maintains a mean trough a4-integrin receptor saturation of greater than 60%.

32. The method according to claim 31, wherein the EID schedule maintains a mean trough α4β1-integrin receptor saturation of greater than 65%.

33. The method according to any one of claims 26-32, wherein the patient is from about 40 kg to about 80 kg in weight and the EID schedule has an interval of at least 5 weeks and no more than 7 weeks.

34. The method of claim 33, wherein the patient is from about 40 kg to about 80 kg in weight and the EID schedule has an interval of 6 weeks.

35. The method of claim 33, wherein the patient is from about 40 kg to about 60 kg in weight and the EID schedule has an interval of from 6 weeks to 7 weeks, preferably 6 weeks.

36. The method according to any one of claims 26-35, wherein the method comprises administering the natalizumab on the EID schedule for at least 6 months, at least 1 year, at least 18 months, at least 2 years, or at least 5 years.

37. A method of improving the safety of chronic natalizumab therapy in a patient in need thereof, comprising determining whether the patient has at least one risk factor for progressive multifocal encephalopathy (PML), and in the presence of said at least one risk factor administering natalizumab to the patient on an EID schedule at a dose of 300 milligrams having an interval of at least 5 weeks and no more than 7 weeks, where the patient is from about 40 kg to about 80 kg in weight.

38. A method of administering to a patient in need thereof a natalizumab therapy, the method comprising: administering the natalizumab therapy on an EID schedule at a dose of 300 milligrams and having an interval of at least 5 weeks and no more than 7 weeks, wherein
a. the patient has a weight range of from 40 kg to less than 80 kg; and
b. the PML risk of the natalizumab therapy is reduced as compared to natalizumab therapy on an SID schedule.

39. A method of improving the safety of chronic natalizumab therapy in a patient in need thereof, comprising determining whether the patient has at least one risk factor for progressive multifocal encephalopathy (PML), and in the presence of said at least one risk factor administering natalizumab to the patient on an EID schedule at a dose equivalent to 3.75 to 7.5 mg natalizumab/kg patient body weight having an interval of at least 5 weeks and no more than 7 weeks.

40. A method of administering to a patient in need thereof a natalizumab therapy, the method comprising: administering the natalizumab therapy on an EID schedule at a dose equivalent to 3.75 to 7.5 mg natalizumab/kg patient body weight and having an interval of at least 5 weeks and no more than 7 weeks, wherein the PML risk of the natalizumab therapy is reduced as compared to natalizumab therapy on an SID schedule.

41. A method of administering to a patient in need thereof a natalizumab therapy, the method comprising: administering the natalizumab therapy on an SID schedule for 12 months, and then administering the natalizumab therapy on an EID schedule.

42. The method of claim 41, wherein the dose amount of the natalizumab therapy on the SID schedule is 300 mg.

43. The method of claim 41 or 42, wherein the dose amount of the natalizumab therapy on the EID schedule is 300 mg.

44. The method of claim 41, wherein the method comprises administering the natalizumab therapy on the EID schedule at a dose equivalent to 3.75 to 7.5 mg natalizumab/kg patient body weight.

45. The method of claim 41 or 44, wherein the method comprises administering the natalizumab therapy on the SID schedule at a dose equivalent to 3.75 to 7.5 mg natalizumab/kg patient body weight.

46. The method of any one of claims 41-45, wherein the EID schedule has an interval of at least 5 weeks and no more than 7 weeks, preferably an interval of at least 5 weeks and no more than 7 weeks.

47. The method of claim 46, wherein the EID schedule has an interval of 6 weeks.

48. The method of any one of claims 41-45, wherein the patient has a weight of less than 120 kg, preferably from about 40 kg to less than 120 kg.

49. The method of claim 48, wherein the patient has a weight of less than 100 kg, preferably from about 40 kg to less than 100 kg.

50. The method of claim 48, wherein the patient has a weight of less than 80 kg, preferably from about 40 kg to less than 80 kg.

51. The method of claim 48, wherein the patient has a weight of less than 60 kg, preferably from about 40 kg to less than 60 kg.
